(19) United States
(12) Patent Application Publication
(10) Pub. No.: US 2025/0232842 A1
(43) Pub. Date: Jul. 17, 2025

(54) METHODS FOR COMPUTATIONAL ANALYSIS OF BIOLOGICAL SAMPLES WITH MACHINE LEARNING ANALYSIS AND SYSTEMS FOR SAME

(71) Applicant: Viome Life Sciences, Inc., Bellevue, WA (US)

(72) Inventor: Brian K. Maples, Palo Alto, CA (US)

(21) Appl. No.: 18/730,737

(22) PCT Filed: Jan. 19, 2023
(86) PCT No.: PCT/US2023/060918

§ 371 Date: Jul. 19, 2024

(51) Int. Cl. G16B 40/20; G16B 40/10; G16H 50/20

(52) U.S. Cl. CPC G16B 40/20; G16B 40/10; G16H 50/20

(57) ABSTRACT

Methods, systems, or computer-readable media are provided for (A) estimating the presence of, or the levels of, analytes in biological samples, (B) estimating characteristics of a condition based on biological samples (C) training a model to estimate a presence of analytes in a biological sample, (D) identifying and evaluating the effectiveness of a treatment for a subject, (E) identifying characteristics of a condition directly based on data obtained from a sample of a subject, and (F) providing a recurring treatment for a subject based on analysis of subject samples.

### CROSS-REFERENCE

This application claims the benefit of U.S. Patent Application No. 63/300,993, filed Jan. 19, 2022, which is hereby incorporated by reference in its entirety

### INTRODUCTION

Diagnosis and treatment of conditions affecting individuals may be improved with enhanced ability to detect the presence of, or in some cases, the levels of, analytes present in biological samples from individuals suspected of having a condition. Mass spectrometry offers techniques for generating data to determine the presence and levels of certain analytes in biological samples at a molecular level. For example, one method of generating data about the presence of analytes in biological samples is coupling liquid chromatography with mass spectrometry-based laboratory techniques, such as tandem-mass spectrometry. However, applying such methods generates a large amount of complex data that can be challenging to analyze and synthesize into diagnoses or treatment options for subjects suspected of having a condition. Computational analysis, such as computational models designed to analyze data generated by mass spectrometry-based laboratory techniques, offers reliable and effective ways to leverage and synthesize data generated by mass spectrometry-based laboratory techniques to improve diagnoses and treatments for individuals. In some cases, a condition affecting an individual is associated with the presence of, or levels of, analytes in a biological sample from the individual, such that improved ability to detect these analytes offers a more reliable way to diagnose and treat the condition. Further, application of computational models can offer faster and cheaper ways to detect a condition and recommend treatments for a condition. In light of their relative speed and affordability, computational models can be applied to data collected using mass spectrometry-based laboratory techniques repeatedly or on a recurring basis. Repeated application of methods for detecting the presence of analytes enables individuals suspected of having a condition to be more closely and more effectively monitored and offers better evaluation of the effectiveness of treatments. Treatment of some conditions, such as, for example irritable bowel disease, may entail dietary interventions, such as providing specific foods for consumption by individuals suspected of having a condition and close monitoring of the effectiveness of the treatment on the individual.

### SUMMARY

Thus, there is a need for improved and useful methods and systems for assessing the presence of, and in some cases, the levels of analytes of interest in biological samples. This invention provides such new and useful methods and systems for training and applying models, such as computational models or machine learning models, to mass spectrometry data. For example, embodiments of the present invention provide model-based methods and systems to (i) estimate the presence of, or levels of, analytes of interest in a biological sample, (ii) characterize a condition of a subject based on estimates and analyses of the presence of, or levels of, analytes of interest in a biological sample, (iii) identify a treatment for a subject based on estimates and analyses of the presence of, or levels of, analytes of interest in a biological sample as well as (iv) evaluate the effectiveness of a treatment for a condition of a subject based on estimates and analyses of the presence of, or levels of, analytes of interest in biological samples. Embodiments may provide such estimates, recommendations or evaluations on a one-time or recurring, i.e., periodic, basis. The invention further relates to methods and systems for training models to provide such estimates, recommendations or evaluations. Other embodiments of the present invention provide model-based methods of directly estimating characteristics of a condition, such as, for example, severity of a disease, from liquid chromatographic and mass spectroscopy data that includes information about the presence of, or levels of, all potential analytes of interest, with or without separately providing individual estimates of the presence of, or levels of, specific analytes, as an intermediate step. Embodiments of the present invention will contribute to making fine grain analysis of conditions, such as medical conditions, that is offered by mass spectrometry-based techniques a more accessible and cost-efficient approach to helping individuals, thereby improving outcomes of individuals suspected of having various conditions. In addition, embodiments of the present invention will enable more detailed analysis of conditions by leveraging the ability to identify and quantitate larger numbers of analytes and signal, in each case on a finer grain basis, from biological samples. Embodiments of the present invention will further contribute to making low-cost treatments, such as food-based interventions, for subjects suspected of having a condition, more effective and available.

Methods and systems for estimating the presence of, or the levels of, analytes in biological samples, as well as training models to make such estimates, are provided. Methods and systems for estimating characteristics of a condition based on biological samples are provided. Aspects of the present invention include methods of training a model to estimate a presence of analytes in a biological sample comprising obtaining liquid chromatographic and mass spectrometry data from the biological sample and possibly other biological samples, selecting training analytes, wherein the training analytes are analytes that may be present in the biological sample, selecting decoy analytes, wherein the decoy analytes are analytes that are not expected to be present in the biological sample, selecting precursors of the training analytes as well as precursors of the decoy analytes, obtaining expected mass-to-charge ratios, predicted retention times and expected relative intensities of isotopes and product ions for each precursor of the training analytes as well as for each precursor of the decoy analytes, preprocessing the liquid chromatographic and mass spectrometry data into one or more arrays, wherein the arrays may be indexed based on mass-to-charge ratio and retention time, generating a tensor data structure for each precursor of the training analytes and for each precursor of the decoy analytes, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursors, and training a model using the tensors corresponding to the precursors of the training analytes and the tensors corresponding to the decoy analytes to estimate a presence of precursors corresponding to analytes.

Aspects of the present invention further include methods of estimating a presence of analytes of interest in a biological sample comprising obtaining liquid chromatographic and mass spectrometry data from the biological sample, selecting the analytes of interest, wherein the analytes of interest are analytes that may be present in the biological sample, selecting precursors of the analytes of interest, obtaining expected mass-to-charge ratios, predicted retention times and expected relative intensities of isotopes and product ions for each precursor of the analytes of interest, preprocessing the liquid chromatographic and mass spectrometry data into one or more arrays, wherein the arrays may be indexed based on mass-to-charge ratio and retention time, generating a tensor data structure for each precursor of the analytes of interest, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor, applying a model trained according to the methods described herein to estimate the presence in the biological sample of the precursors corresponding to the analytes of interest, and inferring the presence of analytes of interest based on estimates of the presence of the precursors corresponding to the analytes of interest.

Also provided are methods of training a model to estimate levels of analytes in a biological sample comprising obtaining liquid chromatographic and mass spectrometry data from the biological sample and possibly other biological samples, selecting training analytes, wherein the training analytes are analytes that may be present in the biological sample, selecting precursors of the training analytes, obtaining expected mass-to-charge ratios, predicted retention times and expected relative intensities of isotopes and product ions for each precursor of the training analytes, preprocessing the liquid chromatographic and mass spectrometry data into one or more arrays, wherein the arrays may be indexed based on mass-to-charge ratio and retention time, generating a tensor data structure for each precursor of the training analytes, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor, and training a model using the tensors corresponding to the precursors of the training analytes to estimate levels of precursors corresponding to analytes.

Also provided are methods for estimating levels of analytes of interest in a biological sample comprising obtaining liquid chromatographic and mass spectrometry data from the biological sample, selecting the analytes of interest, wherein the analytes of interest are analytes that may be present in the biological sample, selecting precursors of the analytes of interest, obtaining expected mass-to-charge ratios, predicted retention times and expected relative intensities of isotopes and product ions for each precursor of the analytes of interest, preprocessing the liquid chromatographic and mass spectrometry data into one or more arrays, wherein the arrays may be indexed based on mass-to-charge ratio and retention time, generating a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor, applying a model trained according to any of the methods described herein to estimate the levels in the biological sample of the precursors corresponding to the analytes of interest, and inferring the levels of analytes of interest based on estimates of the levels of the precursors corresponding to the analytes of interest.

Also provided are methods for characterizing a condition of a subject based on estimates of levels of analytes of interest in a biological sample comprising: obtaining a biological sample from the subject, selecting the analytes of interest, wherein the analytes of interest are analytes that may be present in the biological sample and may be associated with the condition, obtaining estimates of the levels of the analytes of interest in the biological sample by applying methods described herein, and characterizing the condition of the subject based on the estimated levels of the analytes of interest.

Also provided are methods for identifying a treatment for a subject based on estimates of levels of analytes of interest in a biological sample, the method comprising: obtaining a biological sample from the subject, selecting the analytes of interest, wherein the analytes of interest are analytes that may be present in the biological sample, obtaining estimates of the levels of the analytes of interest in the biological sample by applying methods described herein, and identifying the treatment for the subject based on the estimated levels of the analytes of interest in the biological sample.

Also provided are methods for evaluating effectiveness of a treatment for a condition of a subject based on estimates of levels of analytes of interest in biological samples comprising: obtaining a first biological sample from the subject at a first time, selecting the analytes of interest, wherein the analytes of interest are analytes that may be present in the biological sample and may be associated with the condition, obtaining estimates of the levels of the analytes of interest in the first biological sample by applying methods described herein, applying a treatment to the subject, obtaining a second biological sample from the subject at a second time, obtaining estimates of the levels of the analytes of interest in the second biological sample by applying methods described herein, comparing the levels of the analytes of interest in the first and second biological samples, evaluating the effectiveness of the treatment based on the comparison of the levels of the analytes of interest.

Also provided are methods of training and applying a model to estimate characteristics of a condition directly from analysis of biological samples. Aspects of the present invention include methods for training a model to estimate characteristics of a condition, the method comprising: obtaining a first biological sample, wherein the first biological sample is suspected of exhibiting the condition, obtaining a second biological sample, wherein the second biological sample is suspected of not exhibiting the condition, obtaining liquid chromatographic and mass spectrometry data from the first and second biological samples, selecting training analytes, wherein the training analytes are analytes that may be present in the first or second biological samples, selecting precursors of the training analytes, obtaining expected mass-to-charge ratios, predicted retention times and expected relative intensities of isotopes and product ions for each precursor of the training analytes, preprocessing the liquid chromatographic and mass spectrometry data for each of the first and second biological samples into one or more arrays, wherein the arrays may be indexed based on mass-to-charge ratio and retention time, generating a tensor data structure for each precursor of the training analytes for each of the first and second biological samples, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursors, training a model using the tensors corresponding to the precursors of the training analytes of the first and second biological samples to estimate characteristics of the condition.

Aspects of the present invention also include methods for estimating characteristics of a condition of a subject, the method comprising: obtaining a biological sample from the subject, obtaining liquid chromatographic and mass spectrometry data from the biological sample, selecting the analytes of interest, wherein the analytes of interest are analytes that may be present in the biological sample, selecting precursors of the analytes of interest, obtaining expected mass-to-charge ratios, predicted retention times and expected relative intensities of isotopes and product ions for each precursor of the training analytes, preprocessing the liquid chromatographic and mass spectrometry data into one or more arrays, wherein the arrays may be indexed based on mass-to-charge ratio and retention time, generating a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor, and applying a model trained according to methods described herein to estimate characteristics of the condition of the subject.

Also provided are systems for estimating a presence of analytes of interest in a biological sample as well as systems for estimating levels of analytes of interest in a biological sample, as well as training models to make such estimates. Non-transitory computer-readable storage media are also described.

The methods and systems find use in a variety of different applications, e.g., the diagnosis and treatment of subjects suspected of having a condition, such as, for example, irritable bowel disease or non-alcoholic steatohepatitis or Crohn's disease or Rheumatoid arthritis or cardiovascular disease, and the repeated, on-going observation and evaluation of the effectiveness of treatments for individuals suspected of having a condition, such as, for example, diet-based interventions, such as recommending and providing specific foods to subjects.

### BRIEF DESCRIPTION OF THE FIGURES

The invention may be best understood from the following detailed description when read in conjunction with the accompanying drawings. Included in the drawings are the following figures:

FIG. 1 depicts a flow diagram for characterizing a subject's condition using a computational model to analyze a biological sample, according to embodiments of the present invention.

FIG. 2 depicts a flow diagram for developing and updating a treatment for a subject on a recurring bases using a computational model to analyze a biological sample, according to embodiments of the present invention.

FIG. 3 depicts a flow diagram for characterizing a subject's condition and developing a treatment for a subject using a computational model to analyze a biological sample, according to embodiments of the present invention.

FIGS. 4A-4D depict a flow diagram for generating tensors and configuring machine learning models to predict the presence of, or levels of, analytes of interest in data and/or disease or phenotypic status or treatment efficacy derived from applying liquid chromatography-tandem mass spectrometry techniques to a biological sample, according to embodiments of the present invention.

FIGS. 5A-5C depict a flow diagram for creating a tensor data structure, according to embodiments of the present invention.

FIG. 6 depicts a flow diagram for processing a biological sample using a liquid chromatography-tandem mass spectrometry technique, according to embodiments of the present invention.

FIG. 7 depicts an example transition list for a precursor of a protein of interest.

FIG. 8 depicts an example transition list for a decoy protein.

FIG. 9 depicts an exploded view showing how an example tensor data structure is configured, according to embodiments of the present invention.

FIG. 10 depicts a view of an example tensor data structure, according to embodiments of the present invention.

FIG. 11 depicts processing of a tensor data structure by a machine learning model to predict the presence and levels of an analyte of interest, according to embodiments of the present invention.

FIGS. 12A-12B depict a flow diagram for processing liquid chromatography-tandem mass spectrometry to predict presence and levels of analytes of interest in a biological sample, according to embodiments of the present invention.

FIGS. 13A-13B depict examples of training and applying a model to predict characteristics of a condition directly based on one or more biological samples.

FIG. 14 depicts results from an experimental comparison of an embodiment of the present invention for identifying the presence of precursors against methods known in the field.

FIG. 15 depicts results from an experimental comparison of an embodiment of the present invention for identifying the levels of precursors against methods known in the field.

FIG. 16 depicts results from an experimental comparison of an embodiment of the present invention for identifying the levels of precursors against methods known in the field.

FIG. 17 depicts results from an experimental comparison of an embodiment of the present invention for identifying the levels of proteins against methods known in the field.

FIGS. 18A-18B depict results from an experimental comparison of an embodiment of the present invention for identifying the levels of proteins against methods known in the field.

### DETAILED DESCRIPTION

Aspects of the present invention include methods of training a model to estimate a presence of analytes in a biological sample comprising obtaining liquid chromatographic and mass spectrometry data from the biological sample, selecting training analytes, wherein the training analytes are analytes that may be present in the biological sample, selecting decoy analytes, wherein the decoy analytes are analytes that are not expected to be present in the biological sample, selecting precursors of the training analytes as well as precursors of the decoy analytes, obtaining expected mass-to-charge ratios, predicted retention times and expected relative intensities of isotopes and product ions for each precursor of the training analytes as well as for each precursor of the decoy analytes, preprocessing the liquid chromatographic and mass spectrometry data into one or more arrays, wherein the arrays may be indexed based on mass-to-charge ratio and retention time, generating a tensor data structure for each precursor of the training analytes and for each precursor of the decoy analytes, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor, and training a model using the tensors corresponding to the precursors of the training analytes and the tensors corresponding to the decoy analytes to estimate a presence of precursors corresponding to analytes.

Aspects of the present invention further include methods of estimating a presence of analytes of interest in a biological sample comprising obtaining liquid chromatographic and mass spectrometry data from the biological sample, selecting the analytes of interest, wherein the analytes of interest are analytes that may be present in the biological sample, selecting precursors of the analytes of interest, obtaining expected mass-to-charge ratios, predicted retention times and expected relative intensities of isotopes and product ions for each precursor of the analytes of interest, preprocessing the liquid chromatographic and mass spectrometry data into one or more arrays, wherein the arrays may be indexed based on mass-to-charge ratio and retention time, generating a tensor data structure for each precursor of the analytes of interest, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor, applying a model trained according to the methods described herein to estimate the presence in the biological sample of the precursors corresponding to the analytes of interest, and inferring the presence of analytes of interest based on estimates of the presence of the precursors corresponding to the analytes of interest.

Also provided are methods of training a model to estimate levels of analytes in a biological sample comprising obtaining liquid chromatographic and mass spectrometry data from the biological sample and possibly other biological samples, selecting training analytes, wherein the training analytes are analytes that may be present in the biological sample, selecting precursors of the training analytes, obtaining expected mass-to-charge ratios, predicted retention times and expected relative intensities of isotopes and product ions for each precursor of the training analytes, preprocessing the liquid chromatographic and mass spectrometry data into one or more arrays, wherein the arrays may be indexed based on mass-to-charge ratio and retention time, generating a tensor data structure for each precursor of the training analytes, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor, and training a model using the tensors corresponding to the precursors of the training analytes to estimate levels of precursors corresponding to analytes.

Also provided are methods for estimating levels of analytes of interest in a biological sample comprising obtaining liquid chromatographic and mass spectrometry data from the biological sample, selecting the analytes of interest, wherein the analytes of interest are analytes that may be present in the biological sample, selecting precursors of the analytes of interest, obtaining expected mass-to-charge ratios, predicted retention times and expected relative intensities of isotopes and product ions for each precursor of the analytes of interest, preprocessing the liquid chromatographic and mass spectrometry data into one or more arrays, wherein the arrays may be indexed based on mass-to-charge ratio and retention time, generating a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor, applying a model trained according to any of the methods described herein to estimate the levels in the biological sample of the precursors corresponding to the analytes of interest, and inferring the levels of analytes of interest based on estimates of the levels of the precursors corresponding to the analytes of interest.

Also provided are methods for characterizing a condition of a subject based on estimates of levels of analytes of interest in a biological sample comprising: obtaining a biological sample from the subject, selecting the analytes of interest, wherein the analytes of interest are analytes that may be present in the biological sample and may be associated with the condition, obtaining estimates of the levels of the analytes of interest in the biological sample by applying methods described herein, and characterizing the condition of the subject based on the estimated levels of the analytes of interest.

Also provided are methods for identifying a treatment for a subject based on estimates of levels of analytes of interest in a biological sample, the method comprising: obtaining a biological sample from the subject, selecting the analytes of interest, wherein the analytes of interest are analytes that may be present in the biological sample, obtaining estimates of the levels of the analytes of interest in the biological sample by applying methods described herein, and identifying the treatment for the subject based on the estimated levels of the analytes of interest in the biological sample.

Also provided are methods for evaluating effectiveness of a treatment for a condition of a subject based on estimates of levels of analytes of interest in biological samples comprising: obtaining a first biological sample from the subject at a first time, selecting the analytes of interest, wherein the analytes of interest are analytes that may be present in the biological sample and may be associated with the condition, obtaining estimates of the levels of the analytes of interest in the first biological sample by applying methods described herein, applying a treatment to the subject, obtaining a second biological sample from the subject at a second time, obtaining estimates of the levels of the analytes of interest in the second biological sample by applying methods described herein, comparing the levels of the analytes of interest in the first and second biological samples, evaluating the effectiveness of the treatment based on the comparison of the levels of the analytes of interest.

Also provided are methods of training and applying a model to estimate characteristics of a condition directly from analysis of biological samples. Aspects of the present invention include methods for training a model to estimate characteristics of a condition, the method comprising: obtaining a first biological sample, wherein the first biological sample is suspected of exhibiting the condition, obtaining a second biological sample, wherein the second biological sample is suspected of not exhibiting the condition, obtaining liquid chromatographic and mass spectrometry data from the first and second biological samples, selecting training analytes, wherein the training analytes are analytes that may be present in the first or second biological samples, selecting precursors of the training analytes, obtaining expected mass-to-charge ratios, predicted retention times and expected relative intensities of isotopes and product ions for each precursor of the training analytes, preprocessing the liquid chromatographic and mass spectrometry data for each of the first and second biological samples into one or more arrays, wherein the arrays may be indexed based on mass-to-charge ratio and retention time, generating a tensor data structure for each precursor of the training analytes for each of the first and second biological samples, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursors, training a model using the tensors corresponding to the precursors of the training analytes of the first and second biological samples to estimate characteristics of the condition.

Aspects of the present invention also include methods for estimating characteristics of a condition of a subject, the method comprising: obtaining a biological sample from the subject, obtaining liquid chromatographic and mass spectrometry data from the biological sample, selecting the analytes of interest, wherein the analytes of interest are analytes that may be present in the biological sample, selecting precursors of the analytes of interest, obtaining expected mass-to-charge ratios, predicted retention times and expected relative intensities of isotopes and product ions for each precursor of the training analytes, preprocessing the liquid chromatographic and mass spectrometry data into one or more arrays, wherein the arrays may be indexed based on mass-to-charge ratio and retention time, generating a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor, and applying a model trained according to methods described herein to estimate characteristics of the condition of the subject.

Also provided are systems for estimating a presence of analytes of interest in a biological sample as well as systems for estimating levels of analytes of interest in a biological sample, as well as training models to make such estimates. Non-transitory computer-readable storage media are also described.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the system and method may be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

As summarized above, the present disclosure provides methods and systems for estimating the presence of, or the levels of, analytes in biological samples or, in some cases directly estimating characteristics of a condition. By “estimating the presence of an analyte,” it is meant determining whether a specified analyte is detected in a biological sample. For example, in embodiments, estimating the presence of an analyte refers to estimating whether an analyte is present in a biological sample in a quantity that is above a certain threshold. By “estimating the levels of an analyte,” it is meant determining a level, such as a quantity or a quantity relative to other analytes of interest, at which a specified analyte is detected in a biological sample. In embodiments, such determinations are made at least in part based on application of a model, trained or fitted according to the methods described herein, applied to data obtained from laboratory analysis of the biological sample, such as liquid chromatographic and mass spectrometry-based analysis techniques. By “estimating characteristics of a condition,” it is meant estimating qualities of a condition, such as a physiological condition or a medical condition, such as a disease, including, for example, a severity of the condition, identifying the condition, identifying aspects of the condition, identifying mechanisms of the condition, identifying markers related to the condition, such as, for example, degrees of inflammation.

### Methods for Computational Analysis of Biological Samples

Aspects of the present disclosure include methods for estimating the presence of, or the levels of, analytes in biological samples, as well as training models to make such estimates. In particular, the present disclosure includes methods of training a model to estimate a presence of analytes in a biological sample comprising obtaining liquid chromatographic and mass spectrometry data from the biological sample and possibly other biological samples, selecting training analytes, wherein the training analytes are analytes that may be present in the biological sample, selecting decoy analytes, wherein the decoy analytes are analytes that are not expected to be present in the biological sample, selecting precursors of the training analytes as well as precursors of the decoy analytes, obtaining expected mass-to-charge ratios, predicted retention times and expected relative intensities of isotopes and product ions for each precursor of the training analytes as well as for each precursor of the decoy analytes, preprocessing the liquid chromatographic and mass spectrometry data into one or more arrays, wherein the arrays may be indexed based on mass-to-charge ratio and retention time, generating a tensor data structure for each precursor of the training analytes and for each precursor of the decoy analytes, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursors, and training a model using the tensors corresponding to the precursors of the training analytes and the tensors corresponding to the decoy analytes to estimate a presence of precursors corresponding to analytes.

In addition, the present disclosure includes methods of estimating a presence of analytes of interest in a biological sample comprising obtaining liquid chromatographic and mass spectrometry data from the biological sample, selecting the analytes of interest, wherein the analytes of interest are analytes that may be present in the biological sample, selecting precursors of the analytes of interest, obtaining expected mass-to-charge ratios, predicted retention times and expected relative intensities of isotopes and product ions for each precursor of the analytes of interest, preprocessing the liquid chromatographic and mass spectrometry data into one or more arrays, wherein the arrays may be indexed based on mass-to-charge ratio and retention time, generating a tensor data structure for each precursor of the analytes of interest, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor, applying a model trained according to the methods described herein to estimate the presence in the biological sample of the precursors corresponding to the analytes of interest, and inferring the presence of analytes of interest based on estimates of the presence of the precursors corresponding to the analytes of interest.

The present disclosure further includes methods of training a model to estimate levels of analytes in a biological sample comprising obtaining liquid chromatographic and mass spectrometry data from the biological sample and possibly other biological samples, selecting training analytes, wherein the training analytes are analytes that may be present in the biological sample, selecting precursors of the training analytes, obtaining expected mass-to-charge ratios, predicted retention times and expected relative intensities of isotopes and product ions for each precursor of the training analytes, preprocessing the liquid chromatographic and mass spectrometry data into one or more arrays, wherein the arrays may be indexed based on mass-to-charge ratio and retention time, generating a tensor data structure for each precursor of the training analytes, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor, and training a model using the tensors corresponding to the precursors of the training analytes to estimate levels of precursors corresponding to analytes.

In other instances, the method of training a model to estimate levels of analytes in a biological sample comprises obtaining liquid chromatographic and mass spectrometry data from the biological sample, selecting training analytes, wherein the training analytes are analytes that may be present in the biological sample, selecting decoy analytes, wherein the decoy analytes are analytes that are not expected to be present in the biological sample, selecting precursors of the training analytes as well as precursors of the decoy analytes, obtaining expected mass-to-charge ratios, predicted retention times and expected relative intensities of isotopes and product ions for each precursor of the training analytes as well as for each precursor of the decoy analytes, preprocessing the liquid chromatographic and mass spectrometry data into one or more arrays, wherein the arrays may be indexed based on mass-to-charge ratio and retention time, generating a tensor data structure for each precursor of the training analytes and for each precursor of the decoy analytes, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor, training a model using the tensors corresponding to the precursors of the training analytes and the tensors corresponding to the decoy analytes to estimate levels of precursors corresponding to analytes.

In some cases, methods of training a model to estimate levels of analytes in a biological sample comprise obtaining estimates of a presence of the training analytes in the biological sample by applying a second model trained according to the methods described herein, wherein training the model to estimate the levels in the biological sample of the precursors corresponding to the training analytes further comprises training the model using results of estimating the presence of the training analytes.

In addition, the present disclosure includes methods of estimating levels of analytes of interest in a biological sample comprising obtaining liquid chromatographic and mass spectrometry data from the biological sample, selecting the analytes of interest, wherein the analytes of interest are analytes that may be present in the biological sample, selecting precursors of the analytes of interest, obtaining expected mass-to-charge ratios, predicted retention times and expected relative intensities of isotopes and product ions for each precursor of the analytes of interest, preprocessing the liquid chromatographic and mass spectrometry data into one or more arrays, wherein the arrays may be indexed based on mass-to-charge ratio and retention time, generating a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor, applying a model trained according to any of the methods described herein to estimate the levels in the biological sample of the precursors corresponding to the analytes of interest, and inferring the levels of analytes of interest based on estimates of the levels of the precursors corresponding to the analytes of interest.

### Analytes of Interest:

By “analytes of interest,” it is meant any chemical constituent of the biological sample capable of analysis (e.g., measurement or detection) using, for example, mass spectrometry-based analysis techniques. Analytes of interest may consist of chemical components of the biological sample that are or may be associated with a condition, such as a medical condition, and therefore may be used to evaluate whether a subject has such condition. In some cases, analytes of interest may provide other information about a subject, such as markers of inflammation based on the presence or absence of certain analytes of interest. In embodiments, analytes of interest comprise proteins or peptides. In other embodiments, analytes of interest comprise lipids. In still other embodiments, analytes of interest comprise metabolites. By “metabolites,” it is meant any of a variety of chemical compounds produced in connection with a metabolic process. In still other embodiments, analytes of interest comprise polysaccharides. In other embodiments, analytes of interest may comprise still other polymeric substances.

### Training Analytes:

By “training analytes,” it is meant any chemical constituent of the biological sample capable of analysis (e.g., measurement or detection) using, for example, mass spectrometry-based analysis techniques. Training analytes may consist of chemical components of the biological sample. In embodiments, training analytes comprise proteins or peptides. In other embodiments, training analytes comprise lipids. In still other embodiments, training analytes comprise metabolites. By “metabolites,” it is meant any of a variety of chemical compounds produced in connection with a metabolic process. In still other embodiments, training analytes comprise polysaccharides. In other embodiments, training analytes may comprise still other polymeric substances. As a general matter, training analytes may not differ from analytes of interest except that training analytes are used to train a model, whereas models are used to estimate aspects of analytes of interest, such as, e.g., the presence of, or levels of, analytes of interest.

### Biological Samples:

Biological samples of interest may be derived from any organism about which the presence of, or levels of, analytes are sought to be known or about which a condition is to be characterized or a treatment recommended. In some cases, the source of a sample is a “mammal” or “mammalian”, where these terms are used broadly to describe organisms that are within the class mammalia, including the orders carnivore (e.g., dogs and cats), rodentia (e.g., mice, guinea pigs, and rats), and primates (e.g., humans, chimpanzees, and monkeys). In some instances, the source of the samples are humans. The methods may be applied to samples obtained from human subjects of both genders and at any stage of development (i.e., neonates, infant, juvenile, adolescent, adult), where in certain embodiments the human subject is a juvenile, adolescent or adult. While the present invention may be applied to samples originating from a human subject, it is to be understood that the methods may also be carried-out on samples from other animal subjects (that is, in “non-human subjects”) such as, but not limited to, birds, mice, rats, dogs, cats, livestock and horses. It is to be further understood that the methods may also be carried-out on samples from other non-animal subjects such as, but not limited to, plants, fungi, chromista, protozoa, archaea or bacteria.

Biological samples may consist of any aspect of a biological organism capable of isolation and subsequent laboratory analysis via, for example, mass spectrometry-based analysis techniques. For example, in the case of biological samples derived from human subjects, biological samples may consist of, but are not limited to, nasopharyngeal samples, blood samples, saliva samples, urine samples, stool samples, spinal fluid samples, tissue biopsy samples, such as bone marrow samples, or other available tissue samples.

In some embodiments, a biological sample comprises a plurality of biological samples. In some cases, the biological sample comprising a plurality of biological samples is obtained from one or more subjects and further may be obtained at one or more times.

### Decoy Analytes:

Embodiments of the present invention comprise identifying decoy analytes. Decoy analytes may be identical to analytes of interest in all respects except for that decoy analytes are not expected to be present in the biological sample. That is, decoy analytes may be any chemical substance capable of analysis (e.g., measurement or detection) using, for example, mass spectrometry-based analysis techniques, but where decoy analytes are not expected to be present at any level in the biological sample. Decoy analytes may be identified that are known to be not present in the biological sample. In some embodiments, when the biological sample consists of a sample from one organism, decoy analytes may be identified that derive from a different organism, where the organisms are known not to share the decoy analytes. In some embodiments, the decoy analytes are not expected to be present in humans. In other embodiments, the decoy analytes are derived from maize or another non-human organism. That is, for example, when the biological sample is from a human, decoy analytes may be analytes from, for example, maize, where the decoy analytes are known to be not present in humans and therefore not present in a biological sample obtained from a human. In still other embodiments, the decoy analytes are derived from non-human subjects.

Decoy analytes may function as control analytes with respect to training and applying a model in embodiments of the present invention. Specifically, decoy analytes may function as negative controls, since they are known not to be present in the biological sample. In contrast, analytes of interest may be present in the biological sample. The use of negative controls in the form of decoy analytes facilitates training a model according to the present invention, as described in greater detail below, to identify the presence of analytes of interest in biological samples. In embodiments where analytes of interest are proteins or peptides, decoy analytes may, but need not always, be proteins or peptides, known to be not present in the biological sample. In embodiments where analytes of interest are lipids, decoy analytes may, but need not always, be lipids, known to be not present in the biological sample. In embodiments where analytes of interest are metabolites, decoy analytes may be, but need not always be, similar metabolites, known to be not present in the biological sample.

Decoy analytes may be used in connection with training a model to predict the presence of analytes of interest. While this need not always be the case, in some instances, decoy analytes may also be used in connection with training a model to predict levels of analytes of interest.

### Precursors of Analytes:

Embodiments of the present invention comprise identifying precursors of analytes, such as precursors of training analytes, precursors of analytes of interest and precursors of decoy analytes. By “precursor,” it is meant an ion of a component (or other sub-component) of an analyte that forms a part of the training analyte, analyte of interest or decoy analyte. For example, where an analyte is a protein, a precursor of the protein may be a peptide ion that is a section, i.e., a subset, of the amino acid chain that forms the protein analyte of interest, training analyte or decoy analyte along with a charge. That is, precursors may be constituent parts of the analyte of interest, training analyte or decoy analyte such that a precursor, together with other precursors, make up the analyte of interest, training analyte or decoy analyte.

In embodiments, precursors of training analytes, analytes of interest or decoy analytes may be identified in any convenient way using any convenient analytical (e.g., laboratory analysis), computational (e.g., by application of an algorithm or model) or reference (i.e., looking up existing information based on past results or other available information) technique. In certain embodiments, identifying precursors of training analytes, analytes of interest or decoy analytes comprises utilizing previously discovered information, such as, for example, previously discovered data on constituent components of analytes. That is, in such embodiments, identifying precursors comprises looking up a reference (i.e., previously used) precursor of a training analyte, analyte of interest or decoy analyte. In some cases, identifying a precursor by looking up information about previously used precursors of training analytes, analytes of interest or decoys of interest comprises looking up publicly available information about precursors of analytes, such as information on publicly available databases or libraries.

In other embodiments, identifying a precursor of a training analyte, analyte of interest or decoy analyte comprises applying a computational model or algorithm to a representation of the analyte. Such computational model or algorithm may entail identifying common or likely patterns of breaking the training analyte, analyte of interest or decoy analyte into constituent parts along with a charge, i.e., breaking the analyte into precursors. In some cases, identifying precursors comprises conducting an initial laboratory analysis technique with respect to a training analyte, analyte of interest or decoy of interest and recording the results of such laboratory analysis technique for subsequent use identifying a precursor of the training analyte, analyte of interest or decoy analyte by looking up and using such previous result.

In still other embodiments, identifying precursors of training analytes, analytes of interest or decoy analytes comprises applying laboratory techniques to identify constituent components of analytes. In some embodiments, identifying precursors of training analytes, analytes of interest or decoy analytes comprises identifying anticipated products of enzymatic cleavage of analytes. That is, precursors may be identified by identifying the results of applying a treatment, such as an enzymatic or a chemical treatment, expected to cleave the training analyte, analyte of interest or decoy analyte into constituent parts, which constituent parts can then be ionized so that they carry a specific charge, by conducting laboratory analysis, by applying a model, such as a computational model, or by looking up results of previously conducted analysis or previously conducted application of a model. In certain embodiments, identifying precursors of training analytes, analytes of interest or decoy analytes comprises identifying anticipated products of applying a Trypsin digest to the analytes of interest or decoy analytes.

In some cases, for example, when training analytes, analytes of interest or decoy analytes comprise lipids, identifying precursors of analytes comprises ionizing the analyte. That is, in some cases, precursors of analytes comprise charged ions of the analyte and the analyte is not otherwise further broken down into constituent parts (i.e., the relevant constituent component of the precursor is a charged ion of the precursor itself).

### LC-MS/MS Data/Techniques:

Embodiments of the present invention further comprise obtaining liquid chromatographic and mass spectrometry data from the biological sample. Such data comprises liquid chromatographic and mass spectrometry data corresponding to the precursors of the training analytes, analytes of interest as well as decoy analytes and any other analytes when such analytes are present in the biological sample and detectable. Any convenient liquid chromatographic and mass spectrometry-based analytical technique may be applied to generate such data, as such techniques are known in the art. For example, any laboratory technique capable of generating data comprising retention times as well as a mass spectrum indicating the intensities and mass-to-charge ratios of ions of precursor isotopes, adducts, and products of training analytes, analytes of interest and decoy analytes of the biological sample may be employed. In embodiments, a sample may first be processed by liquid chromatography and sample output from the liquid chromatography step is subsequently processed by mass spectroscopy. In other words, a sample output of the liquid chromatography step may be used as a sample input to the mass spectroscopy step. In some cases, the mass spectrographic data comprises results of generating data from a single mass spectroscopy step, sometimes referred to as “MS1.” In other cases, the mass spectrographic data comprises results of generating data first from an initial mass spectroscopy step (MS1), and subsequently from a second mass spectroscopy step, sometimes referred to as “MS2.” In certain cases, the sample input into the second mass spectroscopy step (MS2) comprises results of the sample output by the first mass spectroscopy step (MS1). In embodiments, liquid chromatographic and mass spectrometry data may be generated from, for example, applying liquid chromatography-tandem mass spectrometry (LC-MS/MS) to the biological sample. In embodiments, the liquid chromatographic and mass spectrometry data corresponding to the training analytes, analytes of interest and/or decoy analytes comprises liquid chromatography-tandem mass spectrometry (LC-MS/MS) data.

In embodiments, the liquid chromatographic and mass spectrometry data corresponding to the training analytes, analytes of interest and/or decoy analytes comprises SWATH mass spectrometry data. SWATH refers to a mass spectrometry technique that is known in the art, as described in, for example, C. Ludwig, et al., Data-independent acquisition-based SWATH-MS for quantitative proteomics: a tutorial, Mol Syst Biol. 2018 August; 14(8): e8126 (doi: 10.15252/msb.20178126), incorporated herein by reference.

### Transition List:

In some embodiments, a precursor corresponds to and/or is associated with a transition list for the precursor, wherein a transition list comprises one or more of: an ordered list of isotopes and product ions of the precursor, an identification of whether the precursor corresponds to a training analyte, analyte of interest or decoy analyte, a predicted liquid chromatographic retention time for each isotope and product ion of the precursor, charge information for each isotope and product ion of the precursor, mass information for each isotope and product ion of the precursor, a mass-to-charge ratio for each isotope and product ion of the precursor, and a ranking of expected mass spectrometry intensity data for each isotope and product ion of the precursor. In some embodiments, there is one transition list per precursor per biological sample.

In some embodiments, obtaining transition list values comprises performing a liquid chromatography-tandem mass spectrometry (LC-MS/MS) technique on the biological sample. In other embodiments, obtaining transition list values corresponding to the biological sample comprises applying a computational model to predict a ranking of mass spectrometry intensity data for each isotope and product ion of the precursor. Any convenient computational model may be applied, such as a statistical model, machine learning model, convolutional neural network or the like. In still other embodiments, obtaining transition list values from the biological sample comprises applying a combination of performing a liquid chromatography-tandem mass spectrometry (LC-MS/MS) technique on the biological sample and applying a computational model to predict a ranking of mass spectrometry intensity data for each isotope and product ion of the precursor.

In some embodiments, obtaining transition list values comprises obtaining publicly available data. In other embodiments, obtaining transition list values comprises applying a computational model to predict liquid chromatography retention times and mass spectrometry mass-to-charge-ratios. Any convenient computational model may be applied, such as a statistical model, machine learning model, convolutional neural network or the like. In still other embodiments, obtaining transition list values comprises applying a combination of obtaining publicly available data and applying a computational model to predict liquid chromatography retention times and mass spectrometry mass-to-charge-ratios.

In embodiments, obtaining transition list values comprises identifying or predicting liquid chromatographic retention times using an empirical approach or an iRT-based approach or estimating, for example, using a machine learning approach or a computational model approach or combinations thereof. Any convenient machine learning or a computational model approach may be applied in connection with training analytes, analytes of interest or decoy analytes, such as applying a statistical model, machine learning model, deep learning model, convolutional neural network or the like. Any iRT-based approach, as such are known in the art, may be applied, such as that described in, for example, C. Escher, et al., Using iRT, a normalized retention time for more targeted measurement of peptides, Proteomics. 2012 April; 12(8): 1111-1121 (doi: 10.1002/pmic.201100463), incorporated herein by reference.

### Data Preprocessing:

Embodiments of the present invention comprise preprocessing the liquid chromatographic and mass spectrometry data into one or more arrays, wherein the arrays may be indexed based on mass-to-charge ratio and retention time. In such embodiments preprocessing the data comprises reorganizing or rearranging the data based at least in part on predicted liquid chromatographic retention times and/or expected mass to charge ratios. For example, in some cases, the data is reorganized or rearranged to facilitate inclusion in tensor data structures, such as reorganizing or rearranging the data in order to facilitate excerpting or extracting aspects of the data centered around expected mass-to-charge ratios and retention times of the isotopes and product ions of the precursors.

In embodiments, the liquid chromatographic and mass spectrometry data is associated with a scan type. In such instances, the liquid chromatography and mass spectroscopy instrumentation generates data in increments called scans where each scan is associated with a retention time and contains pairs of mass-to-charge values (m/z) and intensity values. For example, scan 12 might contain pairs <300.0, 100> and <452.1, 250> where an intensity of 100 at m/z 300.0 and an intensity of 250 at m/z 452.1 are observed. In some cases, these scans can be of different scan types depending on what kind of mass spectrometry configuration is used. For example, single mass spectroscopy configuration (i.e., an MS1-type) scans measure the isotopes for the precursors. If a DDA (as such technique is known in the art) configuration is employed, a second, tandem mass spectroscopy configuration (MS2-type) scans cause particular precursors to be fragmented and the resulting product ions are measured. If a SWATH-DIA configuration is employed, the MS2-type scans are actually composed of one type per SWATH window used. For example, if 50 SWATH windows are used, there would be a corresponding 50 scan types, and if MS1-type scans were also employed, 50+1 or 51 scan types would be present. These different scans are often measured in cycles so in the raw mass spectroscopy data there may be an MS1 scan, then an MS2 scan for SWATH window 1, then an MS2 scan for SWATH window 2 and so forth until there would be an MS2 scan for SWATH window 50, and then the scanning process loops back around to an MS1 scan and begins the cycle again. In embodiments, preprocessing the liquid chromatographic and mass spectrometry data comprises de-convolving these raw data scans resulting in grouping the MS1 scans together in a two-dimensional (2D) array, the SWATH window 1 scans together in a 2D array, the SWATH window 2 scans together in a 2D array, and so forth.

When the tensor for a precursor is constructed, embodiments of methods according to the present invention utilize information about where the scan type for an isotope or product ion came from so the method can extract a window from the associated 2D array from the preprocessed data. For example, in embodiments, isotopes are all measured in MS1 scans so the method would just go to that array of preprocessed MS1 scans to extract the desired data. Product ions are slightly more complicated. In SWATH, the scan type is known by looking at the precursor mass and finding the SWATH window with a mass range that covers that mass. This indicates the scan type, e.g., SWATH window 12. In certain embodiments, information about the scan type may be derived from the expected mass to charge ratio of the applicable precursor isotope or product ion.

Embodiments of the present invention comprise obtaining relative intensities of the isotopes and product ions for each precursor of the analytes. Such relative intensities may be included in a transition list associated with a precursor. In such cases, such relative intensities are included in, or associated with, a tensor data structure for a precursor. With respect to the relative intensity, when the precursor is fragmented into product ions as part of the process of obtaining liquid chromatographic and mass spectrographic data, many possible ions result and some will have much higher intensities relative to others, which makes them easier to locate in the data and thus more desirable to look for. In embodiments, a transition list includes a “rank column,” which captures the expected relative intensities of product ions. To actually determine the expected relative intensities there are a few approaches. One approach is to use a machine learning model. Another approach is to run an experiment (either using DDA or DIA mass spectroscopy techniques (as such techniques are known in the art)) and looking at the observed relative intensities. Another approach is to use some subset of the mass spectrographic data obtained from the biological sample. Yet another approach is to use publicly available data. The second approach is most common. For example, in embodiments, an experiment utilizing a DDA mass spectrographic technique may be applied to specifically target and fragment a precursor and observe the isolated product ions that result and obtain their relative intensities from that. In embodiments, an embodiment of a method according to the present invention may make the assumption that these product ions will show a similar pattern of relative intensities in a subsequent application of an embodiment of a method according to the present invention, including, for example, a SWATH experiment.

In embodiments of the present invention, the preprocessed liquid chromatographic and mass spectrometry data comprises transformed intensities. In some cases, preprocessing the liquid chromatographic and mass spectroscopy data into one or more arrays comprises transforming mass spectrographic intensity data. As described in detail above, each scan has a list of <m/z, intensity> pairs. Embodiments of methods according to the present invention may use the mass-to-charge (m/z) value of the <m/z, intensity> pair and the retention time (RT) of the scan for the pair to map the intensity of the pair into a bin in an array (also referred to as a grid). There may be multiple intensities mapped into a bin when the embodiment of the method preprocesses the liquid chromatographic and mass spectrographic data obtained from the biological sample. For example, if in a MS1-type scan at time 100 seconds measurements of <500.1, 1000> and <500.4, 3000> are obtained, and the array grid has a bin for time 90-110 seconds and m/z of 500.0-500.7 then both of the observed intensities would get associated with that bin for the array associated with the MS1 scan type. Intensities for other scan types will also be mapped to bins in the grid and associated with that bin in the array for their own scan type. In the end, a single aggregated intensity per bin in each scan type array is obtained. To obtain this, the method could simply take the average of the observed intensities for a scan type in a bin to get an intensity, which would be 2000 in the example described above. Alternatively, embodiments of methods according to the present invention could take the log of each value before averaging. Alternatively, embodiments of methods according to the present invention could take the average and then log the average. That is, embodiments of the present invention comprise an abstract grid that is used to create the concrete binned or gridded arrays, one array per scan type. Once all of the scans and their <m/z, intensity> pairs are associated with the correct bin in the correct scan type array, and subsequently transformed and aggregated so that there is one transformed/aggregated intensity value per bin per array, the embodiment of the method can proceed to then start extracting windows from these arrays, where the array used is determined by the isotope's or product ion's scan type.

### Tensor Data Structures:

Embodiments of the present invention further comprise generating a tensor data structure for each precursor. In such embodiments, each tensor comprises a three-dimensional array of excerpts of the liquid chromatographic and mass spectrometry data comprising intensity data, such as binned intensity data, for windows around the expected mass-to-charge ratio (m/z) and predicted retention times of the isotopes and product ions of the precursors of the training analytes, analytes of interest and decoy analytes. In addition, tensor data structures may be associated with data indicating whether the tensor relates to a precursor of a training analyte, an analyte of interest or a precursor of a decoy analyte. In embodiments, excerpts of the liquid chromatographic and mass spectrometry data comprise preprocessed liquid chromatographic and mass spectrometry data. In addition, in embodiments, the excerpts of the liquid chromatographic and mass spectrometry data comprise an array of liquid chromatographic and mass spectrometry data centered at the expected mass-to-charge ratio and predicted retention time of isotopes and product ions associated with the precursors of the training analytes, analytes of interest or decoy analytes.

In embodiments, the three-dimensional array of a tensor is configured such that each excerpt of the liquid chromatographic and mass spectrometry data comprising intensity data is centered around the expected location of highest intensity for the analyte isotope or product ion thereof. Different excerpts of the liquid chromatographic and mass spectrometry data may comprise binned intensity data. “Binned” intensity data refers to aggregating intensity data corresponding to, for example, mass spectrographic intensity data over a range of mass-to-charge ratios and retention times for a scan type. In certain embodiments, the liquid chromatographic and mass spectrometry data comprises intensity data for isotopes and/or product ions corresponding to precursors of training analytes, analytes of interest or decoy analytes. In some embodiments, a specified number of excerpts of the liquid chromatographic and mass spectrometry data comprising intensity data are included in a tensor.

In certain embodiments, three-dimensional arrays of tensors comprise a plurality of two-dimensional arrays, wherein each two-dimensional array corresponds to an excerpt of the liquid chromatographic and mass spectrometry data. In such embodiments, the liquid chromatographic and mass spectrometry data may comprise binned intensity data in a window around the expected mass to charge ratio and the predicted retention times of an analyte isotope or product ion. In embodiments, depending on the nature of the liquid chromatographic and mass spectrographic data collected, such excerpts may also comprise intensities of other isotopes or product ions that do not correspond to a training analyte, analyte of interest or decoy analyte, depending on the contents of the biological sample. Where this is the case, training the model comprises training the model to distinguish between intensities of isotopes or product ions corresponding to a training analyte or analyte of interest or decoy analyte and intensities of isotopes or product ions thereof that do not correspond to a training analyte or an analyte of interest or decoy analyte. In such embodiments, the liquid chromatographic and mass spectrometry data comprising intensity data for a window around the expected mass to charge and the predicted retention times of an isotope or product ion may be binned into elements of the corresponding two-dimensional array. As described above, by “binned,” it is meant that the liquid chromatographic and mass spectrometry data comprising intensity data is aggregated, i.e., associated with an array of buckets, each corresponding to a specified range of measurements (such as a range of mass-to-charge ratios and retention times), such that measurements falling within mass-to-charge ratios and retention times corresponding to a particular bucket are associated with that bucket.

In embodiments, the plurality of two-dimensional arrays comprising a tensor is ordered. By “ordered,” it is meant that there is a systematic, repeatable or predictable arrangement of each of the two-dimensional arrays within the tensor. In some embodiments where the plurality of two-dimensional arrays comprising a tensor is ordered, the plurality of two-dimensional arrays of tensors for the training analytes, analytes of interest and decoy analytes are ordered in the same manner. In such embodiments, the plurality of two-dimensional arrays of tensors for the analytes of interest and the decoy analytes may be ordered based on expected mass spectrographic intensities or relative intensities, e.g., where a two-dimensional array that includes the expected greatest intensity isotope or product ion is included in a first position of the tensor data structure, followed by the two-dimensional array that includes the expected second greatest intensity, and so forth.

In embodiments, the plurality of two-dimensional arrays comprising a tensor further comprises a two-dimensional array of weight information. In some cases, the weight information comprises a value at each two-dimensional position corresponding to a distance from a center position of the two-dimensional array of weight information. In such cases, the distance from center of the two-dimensional array may comprise a distance from center based on mass-to-charge ratio. In other cases, the distance from center of the two-dimensional array may comprise a distance from center in liquid chromatographic retention time. In still other cases, the distance from center of the two-dimensional array may comprise a combination of distance from center in mass-to-charge ratio and liquid chromatographic retention time or some other metric used to weight intensity values at different positions of the two-dimensional array.

In embodiments, tensors corresponding to decoy analytes comprise, or are otherwise associated with, information indicating a level of zero in the biological sample—i.e., indicating that the tensor corresponds to a decoy analyte that is not present in the biological sample. In some embodiments, a separate data structure is employed where the separate data structure is configured to track which tensors correspond to decoys or precursors of analytes of interest or training analytes. That is, because decoy analytes are not expected to be present in the biological sample, tensor data structures related to decoy analytes include, or are associated with, for example, using a separate data structure, information indicating the decoy analytes are not present, i.e., their level or quantity in the biological sample is zero.

### Model:

Embodiments of the present invention further comprise training and applying a model. In certain embodiments, the model comprises a statistical model. In some embodiments, the model comprises a linear model. In other embodiments, the model comprises a computational model. In still other embodiments, the model comprises a machine learning model.

In some cases, when the model comprises a machine learning model, the model comprises a tree-based model. In other cases, the model comprises a convolutional neural network. In still other cases, the model comprises an artificial neural network or deep learning network.

In certain cases, the model requires that any input data to the model comply with a specified format. In some cases, tensor data structures, as previously described, may not comply with such required formats. Some embodiments of the present invention, therefore, further comprise transforming the tensor data structures based on the model. That is, the tensor data structures are transformed into a format that is compliant with the requirements of the model but nonetheless retains the information included in, or associated with, tensor data structures.

### Training the Model.

Training a model refers to configuring, fitting or otherwise preparing a model to make predictions, such as, for example, to estimate the presence of, or levels of, analytes of interest and is distinguished from applying a model to make predictions about the presence of, or levels of, analytes of interest. With respect to training a model, embodiments of a model are trained using tensors corresponding to the precursors of training analytes and, in some cases, tensors corresponding to the precursors of decoy analytes to estimate the presence in one or more biological samples of the precursors corresponding to analytes of interest (and, ultimately, the presence of analytes of interest). Training analytes and analytes of interest may be identical to, related to or unrelated to, and completely distinct from, each other. Other embodiments comprise training a model using tensors corresponding to the precursors of training analytes to estimate the levels in one or more biological samples of the precursors corresponding to analytes of interest. Still other embodiments comprise training a model using tensors corresponding to the precursors of training analytes and tensors corresponding to decoy analytes to estimate the levels in one or more biological samples of the precursors corresponding to analytes of interest.

In embodiments, training a model using at least a subset of the tensors comprises applying an unsupervised learning technique to the model. Unsupervised learning is a machine learning technique known in the art for training a model to, for example, identify or recognize patterns. Unsupervised learning comprises training a model where pre-assigned labels are not provided to the model with respect to data used to train the model. That is, in the case of embodiments of the invention, no labels indicating whether a training analyte is present or not in a biological sample are provided in connection with training the model. As a result, applying unsupervised learning to train a model entails the model itself discovering patterns among the training data.

In other embodiments, training a model using at least a subset of the tensors comprises applying a semi-supervised learning technique to the model. Semi-supervised learning is a machine learning technique known in the art for training a model to, for example, identify or recognize patterns. Semi-supervised learning comprises training a model using both labeled and unlabeled training data.

In some embodiments, training a model using at least a subset of the tensors comprises applying a round robin training technique to the model. By “round robin training technique,” it is meant that input data to the model (i.e., data used to train the model) is divided into multiple partitions such that certain partitions are used to train the model and the remaining partitions are used to generate predictions using the trained model. Such process may be iterated where partitions of data previously used to train the model are subsequently used to generate predictions using the trained model. Round robin training approaches may offer benefits including identifying which data sets used for training result in more accurate predictions. Training a model in this context, including by applying unsupervised learning, supervised learning and/or round robin training is described in detail in, for example, L. Reiter, et al., mProphet: automated data processing and statistical validation for large-scale SRM experiments, Nature Methods volume 8, pages 430-435 (2011) (doi.org/10.1038/nmeth.1584), incorporated herein by reference, as well as in V. Demichev, et al., DIA-NN: Neural networks and interference correction enable deep proteome coverage in high throughput, Nat Methods. 2020 January; 17(1): 41-44 (doi: 10.1038/s41592-019-0638-x), incorporated herein by reference.

In embodiments, training the model comprises: initially applying the model to obtain initial predictions, and using at least a subset of the initial predictions to further train the model, wherein initially applying the model comprises obtaining information about the confidence of the prediction generated by the model and the subset of initial predictions used to further train the model correspond to higher confidence predictions. That is, in some embodiments, training the model comprises generating a prediction as well as an indication of the degree of confidence that the prediction is accurate. In some cases, the model itself is configured to generate such an indication of confidence in a prediction.

Some embodiments of the present invention further comprise obtaining weak predictions of the presence of, or levels of, precursors of training analytes or analytes of interest. By “weak predictions,” it is meant an initial prediction, about which limited confidence in the prediction is available. That is, weak predictions may represent only an initial prediction. Such embodiments may further comprise using the weak predictions of the presence of, or the levels of, precursors of training analytes or analytes of interest to train the model to distinguish between analytes of interest and decoy analytes or to predict the levels of precursors. In such embodiments, obtaining weak predictions of levels of precursors of training analytes or analytes of interest comprises preprocessing the liquid chromatographic and mass spectrometry data. In some cases, preprocessing the liquid chromatographic and mass spectrometry data comprises applying an mProphet-based, or DIA-NN-based, data processing technique. DIA-NN-based processing, such as may be applied in connection with the present invention, is described in detail in V. Demichev, et al., DIA-NN: Neural networks and interference correction enable deep proteome coverage in high throughput, Nat Methods. 2020 January; 17(1): 41-44 (doi: 10.1038/s41592-019-0638-x), incorporated herein by reference. Any convenient mProphet-based processing technique known in the art may be applied, such as those described in L. Reiter, et al., mProphet: automated data processing and statistical validation for large-scale SRM experiments, Nature Methods volume 8, pages 430-435 (2011) (doi.org/10.1038/nmeth.1584), incorporated herein by reference. In embodiments that comprise obtaining weak predictions, such embodiments may further comprise associating weak predictions with corresponding tensor data structures.

In some embodiments, weak predictions or estimates of the presence of analytes or precursors of interest can be obtained using, for example, mProphet-based, or DIA-NN-based, techniques, as described above, and a subset of such weak predictions may be selected for use in training a model based on some function or characteristic of these weak predictions or estimates. For example, if weak predictions comprising probabilities of presence in the sample are obtained for analytes or precursors of interest, only those predictions associated with a probability of sufficient confidence (i.e., a confidence above a specified threshold) may be selected for use in training a model.

In some cases, training the model to estimate levels of analytes of interest in a biological sample may comprise first obtaining estimates of the presence of analytes of interest in the biological sample, by for example, the methods described herein, and subsequently utilizing information about the presence of analytes of interest to train a model to estimate levels of analytes of interest in a biological sample. That is, embodiments may be configured to train a model to estimate levels of analytes of interest by taking into account whether or not an analyte of interest is estimated to be present in the biological sample. In particular, embodiments of the present invention for estimating levels of analytes of interest in a biological sample may further comprise obtaining estimates of a presence of the analytes of interest in the biological sample, such as, for example, by applying the methods of estimating the presence of analytes of interest described herein, wherein training the model to estimate the levels in the biological sample of the precursors corresponding to the analytes of interest further comprises training the model using results of estimating the presence of the analytes of interest.

### Applying the Model.

Applying a model refers to using a trained or fitted model to make predictions, such as, for example, to estimate the presence of, or levels of, analytes of interest and is distinguished from training a model as described above. With respect to applying a model, embodiments comprise applying the model to estimate the presence in biological samples of the precursors corresponding to the analytes of interest. Other embodiments comprise applying the model to estimate the levels in biological samples of the precursors corresponding to the analytes of interest. As described above, in embodiments, a trained or fitted model may be applied to tensors corresponding to analytes of interest, which analytes of interest may be different from the training analytes used to train or fit the model. In other words, the model may be trained or fitted to estimate the presence of, or levels of, analytes generally, not exclusively the analytes used to train the model (i.e., the training analytes).

As described in detail above, precursors are ionized constituent parts of analytes of interest. To the extent precursors correspond to analytes of interest, the presence of precursors in a biological sample is indicative of the presence of the corresponding analytes of interest. Similarly, the levels of precursors in a biological sample are indicative of the levels of the corresponding analytes of interest. In some cases, when a single precursor is associated with an analyte of interest (i.e., a tensor data structure is generated for and the model is applied to estimate the presence or level of such a single precursor), information about the presence of, or level of, such precursor for such analyte of interest is used to estimate the presence of, or level of, such analyte of interest. In other cases, when a plurality of precursors is associated with a single analyte of interest (i.e., a plurality of tensor data structures is generated for, and the model is applied to estimate the presence of, or levels of, each of the plurality of precursors), information about the presence of, or levels of, each precursor corresponding to such analyte of interest may be combined to estimate the presence of, or level of, such analyte of interest.

By “estimating the presence of an analyte of interest,” it is meant determining the presence or absence of an analyte of interest in a biological sample. In embodiments, estimating the presence of an analyte comprises estimating whether a particular analyte is detected or is not detected in a biological sample. For example, in embodiments, estimating the presence of an analyte refers to estimating whether an analyte is present in a biological sample in a quantity that is above a certain threshold. In some cases, such threshold refers to an amount of analyte capable of detection in an underlying laboratory analysis technique. For example, in some cases, embodiments of the present invention may detect the presence of an analyte that is present in a biological sample in an amount capable of detection using a mass-spectrometry-based analysis technique, such as, for example, liquid chromatography-tandem mass spectrometry (LC-MS/MS). In some cases, whether an analyte is detectable using LC-MS/MS techniques depends on the amount of analyte present in the biological sample and/or other factors, such as ionization efficiency of the analyte. In other cases, such threshold refers to an amount of analyte corresponding to a meaningful physiological presence in an organism from which the biological sample was obtained.

By “estimating the levels of an analyte of interest,” it is meant predicting a level, such as a quantity, at which a specified analyte is present in a biological sample. For example, embodiments of the present invention may be configured to estimate levels or quantities of an analyte of interest as an amount of mass, e.g., milligrams (or other measure of mass or mass equivalent), as in total estimated milligrams of an analyte in a biological sample or portion thereof. In other cases, embodiments of the present invention may be configured to estimate levels or quantities of an analyte of interest as mass per volume, e.g., milligrams per milliliter (or other measure of mass or mass equivalent and volume or volume equivalent). In other cases, embodiments of the present invention may be configured to estimate relative quantities of an analyte of interest such as relative abundance or relative intensity of a precursor or a subset of isotopes and/or products of a precursor. In still other cases, embodiments of the present invention may be configured to estimate a quantity of an analyte relative to other analytes of interest in the biological sample. For example, embodiments of the present invention may be configured to estimate the equivalent of an ordered list of analytes of interest where the list is ordered according to estimates of the levels of such analytes in a biological sample. Still other embodiments of the present invention may be configured to estimate levels by estimating whether a quantity of an analyte present in a biological sample exceeds a certain threshold. Still other embodiments of the present invention may be configured to estimate levels by setting one analyte's level to an arbitrary number and estimating the levels of other analytes as multiples of that number.

### Other Applications—Identifying Conditions and Treatments Therefor:

Aspects of the present disclosure further include methods for characterizing a condition of a subject based on estimates of levels of analytes of interest in a biological sample. In particular, the present disclosure includes methods for characterizing a condition of a subject based on estimates of levels of analytes of interest in a biological sample comprising obtaining a biological sample from the subject, selecting the analytes of interest, wherein the analytes of interest are analytes that may be present in the biological sample and may be associated with the condition, obtaining estimates of the levels of the analytes of interest in the biological sample by applying methods described herein, and characterizing the condition of the subject based on the estimated levels of the analytes of interest. By “condition,” it is meant any physiological state capable of detection and/or description by, for example, examining the presence of, or levels of, analytes in the subject. By “characterizing a condition,” it is meant gaining understanding of the qualities and aspects of the condition, for example, training models to detect patterns in data generated by LC-MS/MS techniques where such patterns are correlated with conditions of interest or characteristics of such conditions, in each case in a biological sample. In some cases, the condition may refer to a medical condition and characterizing the condition may refer to diagnosing the medical condition in the subject. For example, the condition may refer to irritable bowel disease or non-alcoholic steatohepatitis or Crohn's disease or Rheumatoid arthritis or cardiovascular disease. In other cases, characterizing the condition may refer to understanding aspects of a condition in greater detail, such as understanding the severity of a condition, such as the severity of a medical condition. For example, in some cases, characterizing a condition may refer to measuring a degree of inflammation present. In other cases, characterizing a condition may relate to characterizing how a condition has changed since a prior analysis of the condition, such as a prior estimate of the levels of relevant analytes that were previously estimated according to embodiments of the methods described herein. In other cases, characterizing a condition may relate to characterizing how a condition has been affected by a previously implemented treatment.

Aspects of the present disclosure further include methods for identifying a treatment for a subject based on estimates of levels of analytes of interest in a biological sample and/or the estimated nature of one or more conditions. In particular, the present disclosure includes methods for identifying a treatment for a subject based on estimates of levels of analytes of interest in a biological sample comprising obtaining a biological sample from the subject, selecting the analytes of interest, wherein the analytes of interest are analytes that may be present in the biological sample, obtaining estimates of the presence and/or levels of the analytes of interest in the biological sample by applying methods described herein, and identifying the treatment for the subject based on the estimated levels of the analytes of interest and/or the estimated nature of one or more conditions in the biological sample. Any known or yet to be discovered treatment may be indicated based on the results of obtaining estimates of the levels of the analytes of interest and/or the estimated nature of one or more conditions in the biological sample. In some cases, embodiments of methods according to the present invention may be employed to identify and/or validate previously unknown or not yet tested or validated treatments for a condition.

Treatments identified by embodiments according to the present invention may be food-based treatments or interventions. In some embodiments, a treatment comprises adjusting the subject's diet. In such embodiments, adjusting the subject's diet may comprise instructing the subject to consume a specified food. In other embodiments, adjusting the subject's diet may comprise instructing the subject to consume a specified food supplement. In still other embodiments, adjusting the subject's diet comprises instructing the subject not to consume a specified food. In certain embodiments, adjusting the subject's diet comprises instructing the subject not to consume a specified food supplement. In some cases, a treatment may comprise recommending the subject adhere to a specified diet, such as a specialized diet or a diet consisting of specialized combinations of food or proprietary food-based combinations of nutrients. In still other cases, adjusting the subject's diet comprises instructing the subject to adhere to a specified feeding schedule.

Other treatments besides food-based treatments may be identified by embodiments according to the present invention. In some embodiments, a treatment comprises recommending medication to the subject. In other embodiments, a treatment comprises adjusting the subject's medication. In still other embodiments, a treatment comprises recommending behavior changes to the subject. In still other embodiments, a treatment comprises recommending referral to a specialist. For example, in some instances, application of embodiments of the present invention may entail recommending a referral to a medical specialist, such as, for example, a gastroenterologist or a cardiologist or nutritionist.

Aspects of the present disclosure further include methods for evaluating effectiveness of a treatment for a condition of a subject based on estimates of the presence and/or levels of analytes of interest and/or the estimated nature of one or more conditions in biological samples. In particular, the present disclosure includes methods comprising obtaining a first biological sample from the subject at a first time, selecting the analytes of interest, wherein the analytes of interest are analytes that may be present in the biological sample and may be associated with the condition, obtaining estimates of the presence of, or levels of, the analytes of interest in the first biological sample by applying methods described herein, applying a treatment to the subject, obtaining a second biological sample from the subject at a second time, obtaining estimates of the presence of, or levels of, the analytes of interest in the second biological sample by applying methods described herein, comparing the presence of, or levels of, the analytes of interest in the first and second biological samples, evaluating the effectiveness of the treatment based on the comparison of the presence of, or levels of, the analytes of interest. As described above, in embodiments, methods applied to estimate the presence of, or levels of, analytes of interest may have been trained or fitted using training analytes that differ from the analytes of interest and further may have been trained using training analytes derived from a different biological sample, such as a biological sample not obtained from the subject, such as a different human subject. In some cases, it would be expected that levels of an analyte of interest would change, e.g., go down (or remain constant or go up), after implementation of a treatment, such that a finding that levels had not changed as expected, e.g., gone down (or remained constant or gone up, as the case may be), would be consistent with an accurate understanding of the condition and treatment options therefor. In other cases, it would be expected that levels of an analyte of interest would go down (or remained constant or go up) after implementation of a treatment such that a finding that levels had instead remained constant or gone up (or did not remain constant or gone down, as the case may be) may indicate an inaccurate understanding of the condition and treatment options therefor and suggest that alternative treatment is warranted. In some cases, it is expected that levels of combinations of various analytes of interest may go up or go down or remain constant in an expected pattern after implementation of a treatment. Any convenient amount of time may pass between the first time and the second time biological samples are collected from a subject and may vary depending, for example, on a suspected condition or on the treatment.

Also in particular, the present disclosure includes methods comprising obtaining a first biological sample from the subject at a first time, applying to the first biological sample an embodiment of the methods of the present invention to obtain estimates of the characteristics of one or more conditions in the biological sample, as described herein, applying a treatment to the subject, obtaining a second biological sample from the subject at a second time, applying to the second biological sample an embodiment of the methods of the present invention to obtain estimates of the characteristics of one or more conditions in the biological sample, as described herein, comparing the characteristics of the condition(s) in the first and second biological samples, evaluating the effectiveness of the treatment based on the comparison of the characteristics of the condition(s).

Also provided is a method of estimating characteristics of a condition directly from a model without providing estimates of the presence of, or levels of, individual analytes of interest. With respect to such aspects of the present disclosure, methods of training a model to estimate characteristics of a condition comprise obtaining a first biological sample, wherein the first biological sample is suspected of exhibiting the condition, obtaining a second biological sample, wherein the second biological sample is suspected of not exhibiting the condition, obtaining liquid chromatographic and mass spectrometry data from the first and second biological samples, selecting training analytes, wherein the training analytes are analytes that may be present in the first or second biological samples, selecting precursors of the training analytes, obtaining expected mass-to-charge ratios, predicted retention times and expected relative intensities of isotopes and product ions for each precursor of the training analytes, preprocessing the liquid chromatographic and mass spectrometry data for each of the first and second biological samples into one or more arrays, wherein the arrays may be indexed based on mass-to-charge ratio and retention time, generating a tensor data structure for each precursor of the training analytes for each of the first and second biological samples, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursors, training a model using the tensors corresponding to the precursors of the training analytes of the first and second biological samples to estimate characteristics of the condition.

In addition, aspects of the present disclosure include methods of estimating characteristics of a condition of a subject comprising obtaining a biological sample from the subject, obtaining liquid chromatographic and mass spectrometry data from the biological sample, selecting the analytes of interest, wherein the analytes of interest are analytes that may be present in the biological sample, selecting precursors of the analytes of interest, obtaining expected mass-to-charge ratios, predicted retention times and expected relative intensities of isotopes and product ions for each precursor of the training analytes, preprocessing the liquid chromatographic and mass spectrometry data into one or more arrays, wherein the arrays may be indexed based on mass-to-charge ratio and retention time, generating a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor, and applying a model trained according to methods described herein to estimate characteristics of the condition of the subject. That is, embodiments of the present invention comprise training a model to estimate characteristics of a condition exhibited in a biological sample and using the model to estimate characteristics of a condition exhibited in a biological sample, in each case, without an intermediate step of providing estimates regarding any specific analyte of interest, such as, e.g., the presence of, or level of, an analyte of interest.

### A Recurring Food-Based Treatment:

Aspects of the present disclosure further include methods for treatment for a subject suspected of having a condition. In particular, the present disclosure includes methods comprising: (a) selecting analytes of interest, wherein the analytes of interest may be associated with the condition, (b) obtaining a biological sample from the subject, (c) obtaining estimates of the presence of, or the levels of, the analytes of interest in the biological sample by applying an embodiment of the methods according to the present invention, (d) identifying the treatment for the subject based on the estimated presence of, or levels of, the analytes of interest in the biological sample, (e) recommending the treatment to the subject for a specified period of time, (f) providing recurring evaluation and treatment for the subject by repeating steps (a) through (e) one or more times. In embodiments, analytes of interest can include compounds known to be associated with a condition, such as a disease, or hypothesized to be associated with a condition. For example, in some cases, analytes of interest could be the entire human proteome, or subsets thereof. In embodiments of such method, training a model used to estimate the presence of, or levels of, analytes of interest could occur in different ways, such as those described above, including training a model using other biological samples (i.e., one or more biological samples obtained from one or more organisms that are not the subject) and applying such trained model to the biological sample obtained from the subject. Embodiments of such method may comprise a service wherein a subject suspected of having a condition is periodically evaluated by estimating the presence and/or levels of certain analytes, and a treatment plan is continually updated based on the estimated presence and/or levels of analytes.

In embodiments, the recurring intervention comprises a subscription service. In some cases, a subscription service may refer to a payment that includes a specified number of periodic recurring interventions or continual periodic interventions until some other condition is satisfied, such as specific analyte levels, characteristics of the condition, i.e., resolution of the condition, or specific functionality, ability or general wellness gained by the subject. In other cases, subscription service refers to recurring recommendations for treatment of the condition, such as providing a treatment on a recurring basis.

In other embodiments, identifying the treatment for the subject comprises identifying changes to the subject's diet. In such embodiments, recommending the treatment to the subject may comprise providing food-based treatment to the subject. In embodiments, providing food-based treatment to the subject comprises a food subscription service. By food subscription service, it is meant a subscription service that provides food-based treatments to the subject on a recurring, periodic basis. In other embodiments, the recurring intervention for the subject is repeated on a periodic basis, wherein the period is determined at least in part based on the estimated presence and/or levels of the analytes of interest in the biological sample. That is, in the event estimated presence and/or levels of certain analytes are trending toward a normal range, the period of time between collecting and analyzing biological samples from the subject may be increased. In certain embodiments, the suspected condition is irritable bowel disease or non-alcoholic steatohepatitis or Crohn's disease or Rheumatoid arthritis or cardiovascular disease.

### Multiple Biological Samples:

In embodiments of the methods described herein, models may be trained using liquid chromatographic and mass spectrometry data obtained from a single biological sample or, in other cases, from a plurality of biological samples. In some cases, the plurality of biological samples comprise distinct biological samples obtained from one or more subjects at one or more times. In some cases, models initially trained using liquid chromatographic and mass spectrometry data obtained from one or more biological samples may subsequently be further trained using further liquid chromatographic and mass spectrometry data obtained from one or more further biological samples. In embodiments in which a plurality of biological samples is used to train a model, tensor data structures may be generated for precursors corresponding to one or more of the biological samples used to train the model.

### Exemplary Embodiment—Characterizing a Condition

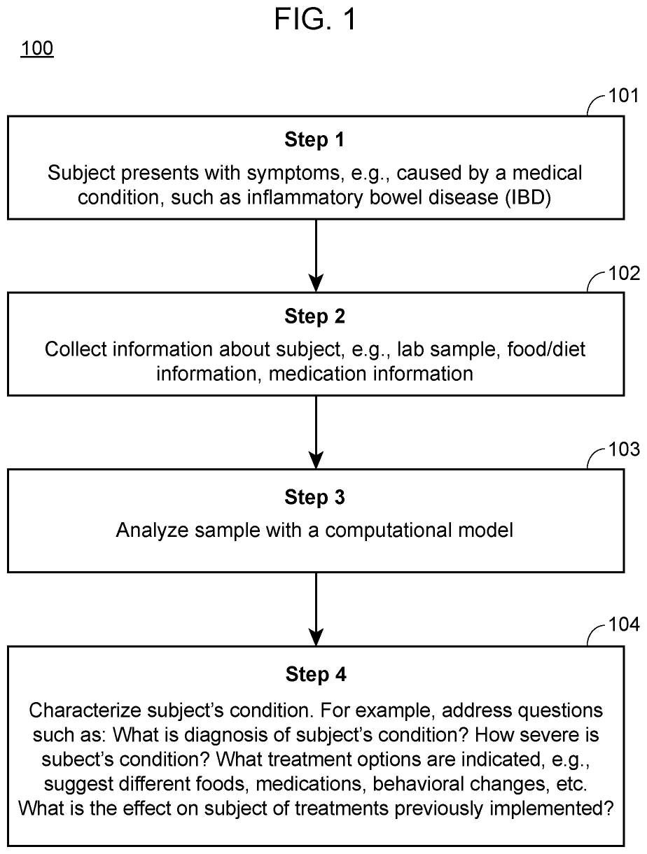

FIG. 1 illustrates a flow diagram for characterizing a condition according to some aspects of the present disclosure 100. At block 101 of exemplary embodiment of method for characterizing subject's condition 100, a subject is suspected of having a condition, for example, a subject presents with suspected possible symptoms of a potential medical condition, such as, for example, irritable bowel disease or non-alcoholic steatohepatitis or Crohn's disease or Rheumatoid arthritis or cardiovascular disease.

At block 102, information is collected about the subject. Such information may comprise interview information, e.g., a description of the suspected symptoms, a description of the subject's medical history or a description of factors that may be related to the suspected medical condition. In addition, such information may comprise collecting a sample in a laboratory, i.e., a biological sample, such as, for example, a blood sample. In addition, such information may comprise information about any current or on-going treatments, such as, for example, specific food or diet information or prescription medicine used. Such information may be used to in part select analytes of interest to observe in the biological sample or to identify or rule out suspected medical conditions or potential applicable treatments.

At block 103, the biological sample collected at block 102 is analyzed using a computational model, such as a model according to embodiments of the present invention. In block 103, the computational model may be applied to estimate the presence of, or levels of, analytes of interest that have bearing on characterizing a subject's condition or for directly characterizing a condition or aspects of a condition in the patient.

At block 104, the subject's condition is characterized based at least in part on the results of analysis by the computational model at block 103. That is, the estimated presence of, or levels of, analytes in the biological sample may be applied at block 104 to characterize the subject's condition, such as, for example: to diagnose the subject's condition; to evaluate the severity of the subject's condition, such as, for example, estimating levels of inflammation; determine what treatment options may be indicated, such as, for example, by suggesting different foods or diet plan or medications or behaviors to the subject. In some cases, the estimated presence of, or levels of, analytes in the biological sample may be used to evaluate the effect that previously implemented treatments, such as a food or diet plan that is current in effect, have had on the subject. In some cases, the subject's condition may be characterized at block 104 as being normal, meaning that the presence of, or levels of, analytes of interest in the biological sample are within a normal range. In other cases, the estimated characterization of the subject's condition may be made directly from the biological sample, i.e., tensor data derived from the liquid chromatographic and mass spectrographic data of the biological sample and possibly other data collected using a computational model, without estimating or evaluating individual analytes of interest.

### Exemplary Embodiment—Recurring Evaluation of Treatment for a Condition

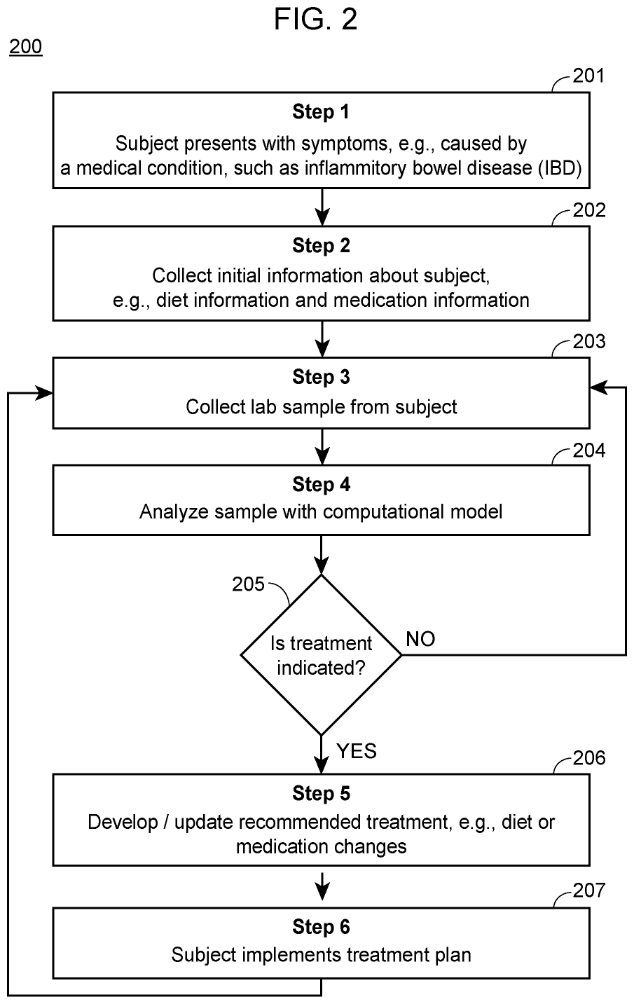

FIG. 2 illustrates a flow diagram for evaluating treatment for a condition according to some aspects of the present disclosure 200. At block 201 of exemplary embodiment of method for evaluating treatment for a condition 200, a subject is suspected of having a condition, for example, a subject presents with suspected possible symptoms of a potential medical condition, such as, for example, irritable/inflammatory bowel disease or non-alcoholic steatohepatitis or Crohn's disease or Rheumatoid arthritis or cardiovascular disease.

At block 202, information is collected about the subject. Such information collection step may be identical to that described above in connection with block 102 of FIG. 1. Similarly, such information collected at block 202 may be used in part to select analytes of interest to observe in a biological sample from the subject or to identify or rule out suspected medical conditions or potential applicable treatments. However, in some cases, analytes of interest may be chosen for discovery applications, for example the entire human proteome, lipidome, metabolome, or some combination of subsets thereof may be selected as analytes of interest.

At block 203, a biological sample is collected in a lab from the subject. Such biological sample may be, for example, a blood sample collected from a blood draw. In some cases, the type of sample collected at block 203 is determined based at least in part on the information, and therefore associated potential conditions and related treatments, collected at block 202.

At block 204, the biological sample collected at block 203 is analyzed using a computational model, such as a model according to embodiments of the present invention, to estimate the presence of, or levels of, analytes of interest in the biological sample or for characterizing conditions in the patient. Analysis at block 204 may be identical to the that described in connection with block 103 of FIG. 1.

At block 205, a determination is made based at least in part on the estimations of the presence of, or levels of, analytes in the biological sample, or the estimated characterization of a condition of the subject, of whether a treatment is indicated. Such determination may be made based on conditions such as, for example, the levels of certain analytes associated with a condition and/or a specific treatment or the estimated nature or characterization of the condition and/or other conditions of the subject. For example, if the presence of, or levels of, analytes of interest estimated at block 204 are within normal ranges, treatment may not be indicated, and the process moves to block 203 next. In the event the process moves to block 203, some time may be allowed to pass before collecting a lab sample again at block 203. If, on the other hand, estimated levels of analytes of interest associated with a condition, such as, for example, irritable bowel disease or non-alcoholic steatohepatitis or Crohn's disease or Rheumatoid arthritis or cardiovascular disease, indicate such condition may be present in the subject, treatment may be warranted, and the process moves to block 206 next.

At block 206, a recommended treatment is developed based at least in part on the results of estimating the presence of, or levels of, analytes of interest or the estimated nature or characterization of a condition or conditions of the subject in block 204 and the related determination of whether further treatment is warranted at block 205. In some cases, when a treatment program is already in place for the subject, at block 206, the treatment program may be updated based at least in part on the results of estimating the presence of, or levels of, analytes of interest or the estimated nature or characterization of a condition or conditions of the subject in block 204 and the related determination of whether further treatment is warranted at block 205. By updating the treatment program, it is meant adjusting the treatment program, such as for example, increasing or decreasing existing levels of recommended treatment, or, in other cases, canceling aspects of the treatment or adding new treatment options. At block 207, the subject continues to implement the treatment program developed and/or updated at block 206. The subject may continue implementing the treatment program for a specified period of time, which period may depend on the treatment and/or the condition and/or the presence of, or levels of, analytes of interest present in the subject or the estimated nature of a condition or conditions of the subject, before the process returns to block 203 for further evaluation of the subject's condition.

### Exemplary Embodiment—Identifying Treatments for a Condition

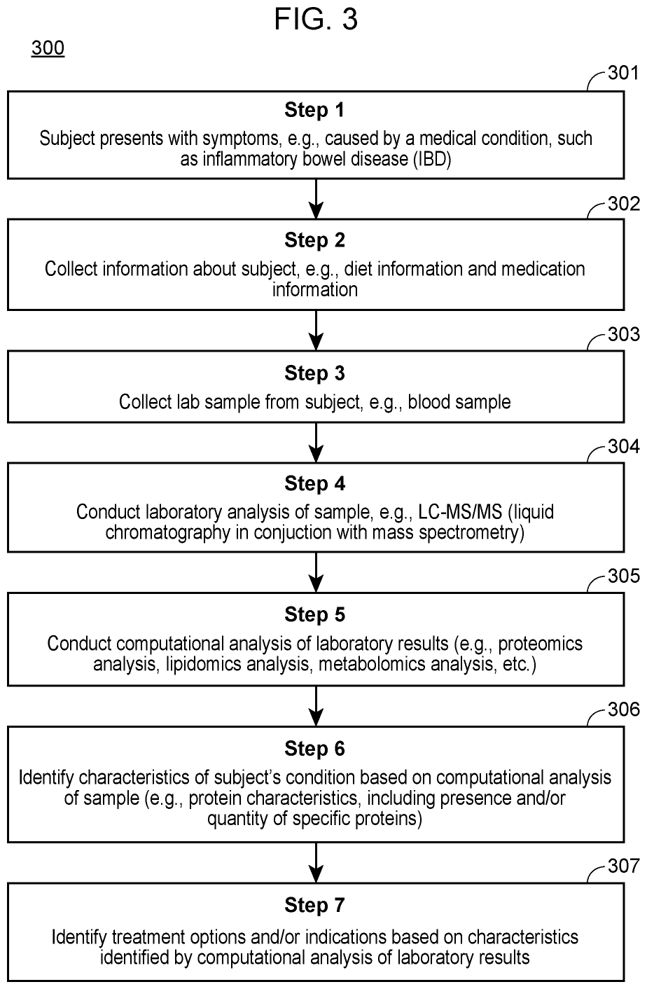

FIG. 3 illustrates a flow diagram for identifying treatment options for a subject's condition according to some aspects of the present disclosure 300. At block 301 of exemplary embodiment of method for identifying treatment options 300, a subject is suspected of having a condition, for example, a subject presents with suspected possible symptoms of a potential medical condition, such as, for example, irritable/inflammatory bowel disease or non-alcoholic steatohepatitis or Crohn's disease or Rheumatoid arthritis or cardiovascular disease.

At block 302, information is collected about the subject. Such information collection step may be identical to that described above in connection with block 102 of FIG. 1 and/or block 202 of FIG. 2. At block 303, a biological sample is collected in a lab from the subject. Such sample collection step may be identical to that described above in connection with block 102 of FIG. 1 or block 203 of FIG. 2.

At block 304, the biological sample collected at block 303 is subjected to laboratory analysis techniques to provide the raw data on which a computational model according to the present invention operates. At block 304, liquid chromatography may be applied to the sample as a separation technique. The sample, having been separated via liquid chromatography, may then be subjected to multiple applications of mass spectrometry to obtain mass-charge information about the separated sample. That is, liquid chromatography tandem mass spectrometry analysis technique may be applied to the biological sample to obtain intensity data related to the contents of the biological sample, where such intensity data may also include data capable of indicating the presence (or absence) of, or level of, constituent isotopes, adducts, and product ions of precursors of analytes of interest present (or not present) in the sample. Such data may in some cases take the form of intensity data over a two-dimensional grid with axes representing mass-charge ratio and retention time.

At block 305, computational analysis is applied to the results of the laboratory analysis techniques obtained at block 304, such as, for example, mass-charge and retention time intensity data. Depending on what analytes of interest are being examined, computational analysis applied at block 305 may comprise proteomics analysis, lipidomics analysis, metabolomics analysis or the like. Application of the computational analysis at block 305 results in estimates of the presence of, or levels of, analytes of interest, such as proteins or lipids or metabolites or the like or direct estimates of the nature or characterization of a condition or conditions of the subject.

At block 306, characteristics of the subject's condition are identified based on the results of applying computational analysis at block 305. In particular, the subject's condition may be identified based on the presence of, or levels of, analytes of interest that were identified through computational analysis at block 305 of the laboratory results obtained at block 304, or from the direct estimation of the nature of one or more conditions of the subject. Relevant characteristics of the subject's condition may include, for example, the presence of certain proteins that are considered, or are found, to be indicative of a condition or a severity of a condition or indicative of a specific treatment for a condition. Another exemplary characteristic of the subject's condition is an estimated characterization of one or more conditions where such estimation is made directly from analysis of the sample.

At block 307, treatment options for the subject are identified based on characteristics of the subject's condition identified at block 306 based on the results of computational analysis at block 305 of the laboratory results obtained at block 304. That is, for example, the levels of certain analytes of interest or the characterization of one or more conditions may indicate a certain treatment is warranted. For example, as described in detail above, treatment options may comprise food-based interventions, such as recommending the subject consume certain foods or food supplements for a specified period of time.

### Exemplary Embodiment—Estimating Presence or Levels of Analytes

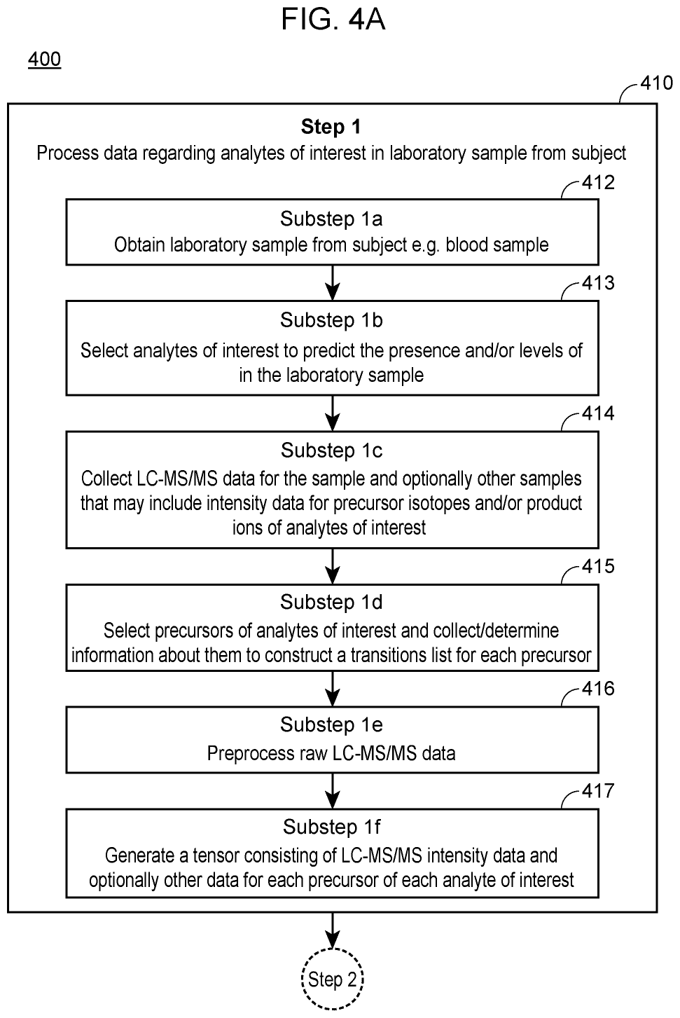

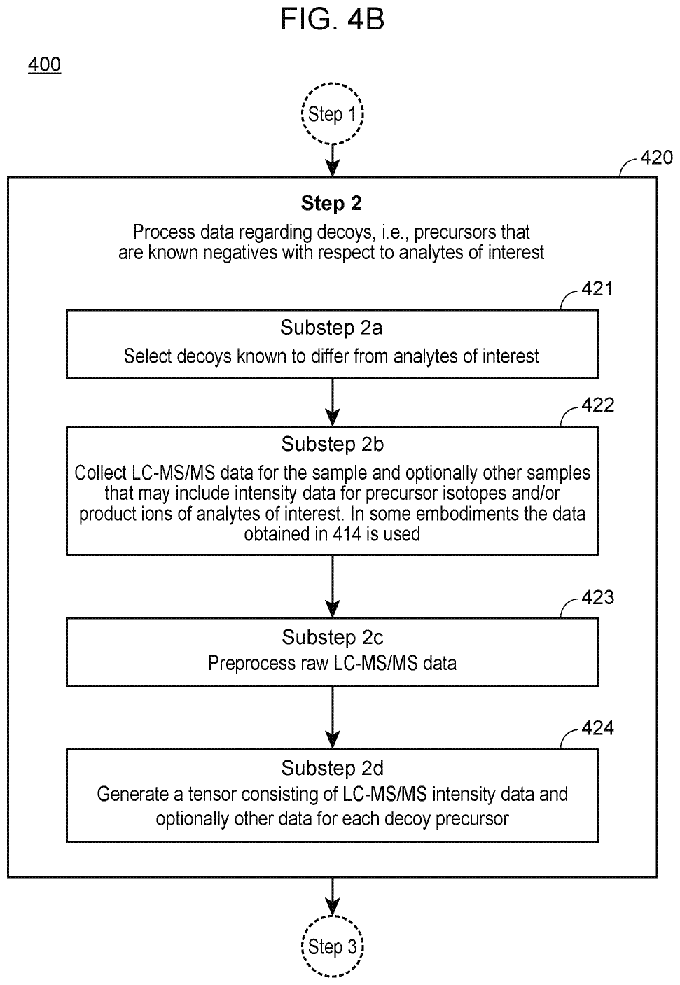

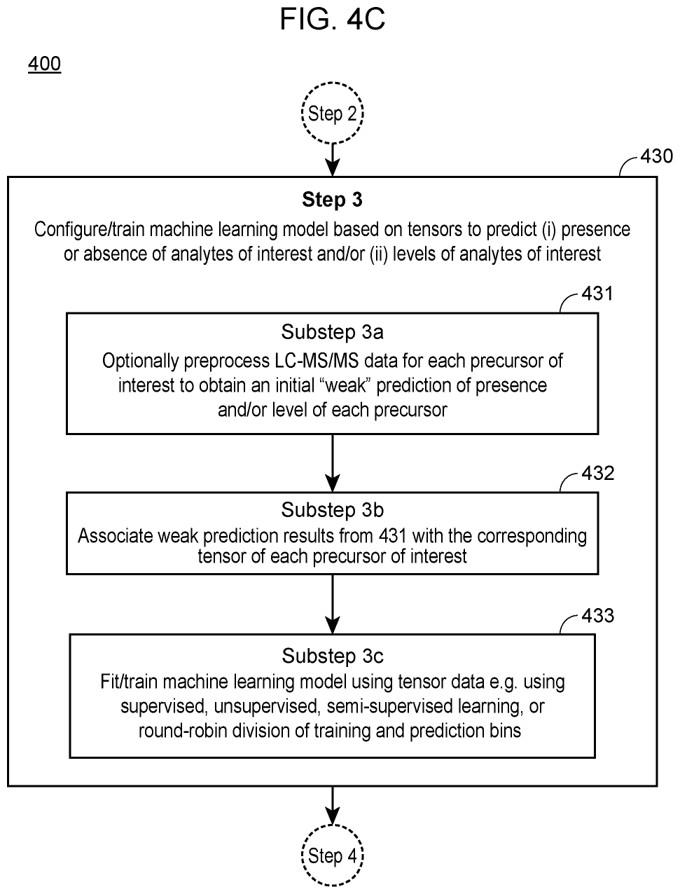

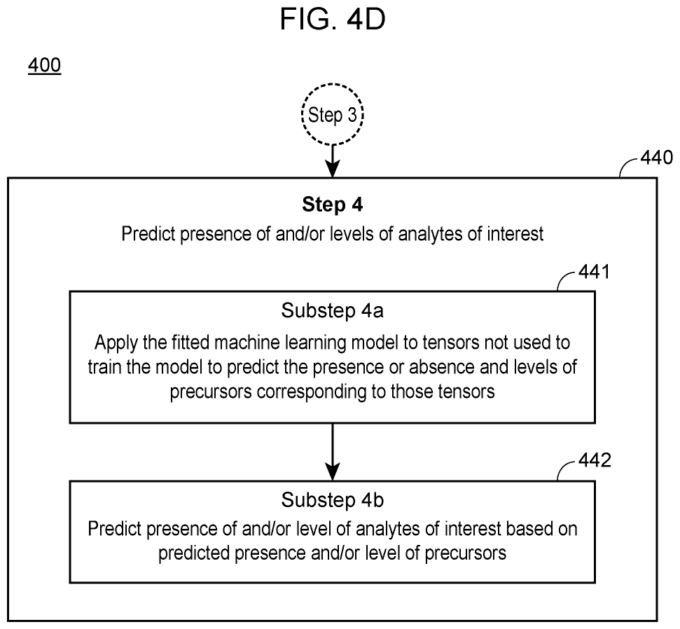

FIGS. 4A-4D illustrate a flow diagram for estimating the presence of, or levels of, analytes of interest in a biological sample according to some aspects of the present disclosure 400.

In FIG. 4A, block 410 of exemplary embodiment of method for estimating the presence of, or levels of, analytes of interest 400, comprises processing data from a biological sample from a subject. Block 410 is comprised of substeps 412, 413, 414, 415, 416 and 417. At block 412, a laboratory sample (i.e., a biological sample) is obtained from a subject. Such sample collection step may be identical to that described above in connection with block 102 of FIG. 1, block 203 of FIG. 2 or block 303 of FIG. 3.

At block 413, analytes of interest are selected. As described in detail above, analytes of interest are analytes that are potentially present in the biological sample and about which the embodiment is configured to predict the presence of, or levels of Analytes of interest may be selected based on, for example, a condition that the subject is suspected of having or a condition to be ruled out. Another example would be selecting all analytes in a proteome, lipidome and/or metabolome to discover novel indicators of disease. In some cases, a standard list of analytes of interest are always selected.

At block 414 liquid chromatography and mass spectrometry laboratory analysis data is collected for the sample. Specifically, liquid chromatography tandem mass spectrometry data is obtained that may include intensity data for isotopes, adducts, and product ions of each precursor. That data obtained may be LC-MS or LC-MS/MS data with the latter being DDA or DIA including DIA-SWATH, as such mass spectrographic techniques are described in detail below.

At block 415, precursors of the selected analytes of interest are selected and information determined. As described above, a precursor may be any part or subset or fragment of an analyte. Precursors may be selected based on their uniqueness in analytes of interest, their likelihood of detection by the selected LC-MS or LC-MS/MS laboratory method, information available about them, or for other reasons. In connection with selecting each precursors of interest, information about isotopes and product ions are also collected or determined. This information includes the precursor's expected mass-to-charge-ratio, the expected mass-to-charge ratio of each isotope and product ion, the expected retention time of the precursor and each isotope and product ion, the relative intensity of each product ion, and optionally other attributes such as ionization efficiency. This information can be used to select which isotopes and product ions to use for creation of the tensors and the relative ordering of the isotopes and product ions within the tensor.

At block 416, the raw LC-MS/MS data is preprocessed. LC-MS/MS data can be thought of as a two-dimensional image or a set of two-dimensional images with one dimension indexed by mass-to-charge ratio and the other dimension indexed by retention time or scan index. Each scan is associated with a retention time by the LC-MS/MS instrumentation and software. For LC-MS data with only MS1 scans, each sample data can be represented by a single image. For SWATH LC-MS/MS data with N MS2 windows and MS1 measurements there are N+1 images, where one image corresponds to the aggregation of MS1 scans and each of the other N images are comprised of the scans corresponding to a SWATH window. Other approaches to LC-MS/MS can be considered in a similar fashion. The values in the image correspond to observed intensities at measured mass-to-charge ratios (m/z) in each scan. Scans corresponding to MS1 are aggregated and similarly each of the N MS2 windows are aggregated if present. An M×T grid is selected that is composed of M possibly overlapping ranges of m/z values as well as T possibly overlapping ranges of retention time values. For each MS2 window, if measured, and the MS1 measurement, if measured, an M×T tensor data structure is initialized with values of 0 in each element. For each observed m/z value in each scan of the MS1 scans, if present, the grid location or locations in the associated tensor is determined and the measured intensity is associated with that location. This is similarly done for each of the MS2 windows if present. Finally, all intensities associated with a particular grid location in each tensor are aggregated in some way, which could include taking an average, the median value, the maximum value, or some other calculation. Transformations of values may be applied before or after aggregation and can include applying logarithm or tangent functions or other functions that are known in the art.

At block 417, excerpts of the preprocessed LC-MS/MS data obtained at block 416 are used to generate tensor data structures. One tensor data structure is generated for each precursor selected in block 415. Tensor data structures include excerpts of the preprocessed LC-MS/MS data, such as SWATH data, where the excerpts correspond to windows centered around the expected m/z and retention times of isotopes of precursor molecules or product ions of precursor molecules, as such isotopes and product ions may be produced as a result of the mass spectrometry analysis in the LC-MS/MS technique.

FIG. 4B depicts steps subsequent to block 410 of the exemplary embodiment of method for estimating the presence of, or levels of, analytes of interest 400. In FIG. 4B, the method proceeds to block 420 which comprises processing data regarding decoy analytes (i.e., decoys). As described above, decoy analytes are used in connection with embodiments of the present invention for predicting the presence of analytes of interest and may not be typically used in connection with embodiments of the present invention for predicting levels of analytes of interest. Decoy analytes are identical to analytes of interest except that decoy analytes are known not to be present in the biological sample. For example, in the event a biological sample is obtained from a human subject, a decoy analyte may originate from maize, where the decoy analyte is known not to be present in human subjects. That is, decoy analytes are known negatives with respect to the biological sample; i.e., decoy analytes function as negative controls with respect to identifying the presence of analytes of interest in a biological sample.

At block 421, decoy analytes, as well as precursors therefor, are selected. Decoy analytes may be selected in any convenient manner, such as, for example, by looking up potential decoy analytes in reference materials, such as, for example, online libraries or databases. Alternatively, decoy analytes may be computationally generated such that their precursors typically have similar characteristics such as similar m/z and retention time ranges as precursors of analytes of interest without sharing the same chemical structure or sequence. Decoy analytes, by definition, are different molecules than analytes of interest. Further, where analytes of interest may be present in a biological sample, decoy analytes are known or can be assumed not to be present in the biological sample.

At block 422, liquid chromatography mass-spectrometry data is obtained for one or more samples and such data may be the same data that were previously obtained for the precursors of analytes of interest. Specifically, LC-MS/MS data is obtained in the same form as the LC-MS/MS data obtained with respect to analytes of interest at block 414. That is, the LC-MS/MS data with respect to decoy analytes obtained at block 422 is analogous to the LC-MS/MS data with respect to analytes of interest at block 414. In some cases, the LC-MS/MS data obtained regarding decoy analytes is SWATH data or excerpts of SWATH data. With respect to obtaining LC-MS/MS data for decoy analytes, such data may be obtained based on previously collected data.

At block 423, the data collected in block 422 are preprocessed in a similar fashion as that described in connection with block 416, as described above. Often, the same preprocessed data from block 416 will be used at block 423.

At block 424, tensor data structures are generated for precursors of decoy analytes identified at block 421 using excerpts of the preprocessed LC-MS/MS data obtained at block 423. Generating tensor data structures with respect to precursors of decoy analytes is analogous to generating tensor data structures with respect to precursors of analytes of interest at block 417. In particular, one tensor data structure is generated for each precursor. Tensor data structures include excerpts of the preprocessed LC-MS/MS data, such as SWATH data, where the excerpts correspond to windows of preprocessed LC-MS/MS data centered around the expected location of intensity, if any, regarding isotopes of precursor molecules or product ions of precursor molecules, as such isotopes and product ions are produced as a result of the mass spectrometry analysis in the LC-MS/MS technique.

FIG. 4C depicts steps subsequent to block 420 of the exemplary embodiment of method for estimating the presence of, or levels of, analytes of interest 400. In FIG. 4C, the method proceeds to block 430 which comprises training one or more machine learning models to predict the presence of analytes of interest in a biological sample and/or to estimate levels of analytes of interest in a biological sample. While this need not always be the case, typically, one machine learning model would be trained to predict the presence of analytes of interest in a biological sample and a separate machine learning model would be trained to estimate levels of analytes of interest in a biological sample. Training a machine learning model to predict the presence of analytes of interest in a biological sample may involve using tensors corresponding to precursors of training analytes, which may be a subset of, or distinct from, the analytes of interest, as well as precursors corresponding to decoy analytes to train the model. Training a machine learning model to estimate levels of analytes of interest in a biological sample may involve using only tensors corresponding to precursors of training analytes to train the model or may involve using tensors corresponding to precursors of training analytes as well as precursors corresponding to decoy analytes to train the model.

At block 431, LC-MS/MS data that includes data, if any, regarding precursors of analytes of interest obtained at block 414 may optionally be preprocessed to obtain initial predictions about the presence of, or levels of, the precursors corresponding to analytes of interest. Initial predictions may be referred to as “weak” predictions to indicate such predictions are generated not by the full embodiment of a method according to the present invention but by, for example, known tools in the art, such as an mProphet-based approach, as described above, or an mProphet-based approach in conjunction with a Skyline-based approach, as such tool is known in the art and described, for example, in B. MacLean, Skyline, Bioinformatics, Volume 26, Issue 7, April 2010, pp 966-968 (https://doi.org/10.1093/bioinformatics/btq054), incorporated herein by reference. Similarly, “weak” predictions for the presence of precursors of interest can also be generated and used to include or exclude precursors of interest for training models. For example, all precursors with a weak prediction regarding its presence being less than 50% may be excluded from subsequent training or initial rounds of training, though other thresholds or scoring schemes can be used.

At block 432, the “weak” prediction results obtained at block 431 are associated with corresponding tensor data structures that represent the precursors of analytes of interest, about which “weak” predictions were obtained. Such prediction results may be stored in a data structure that associates the prediction results with the relevant tensor in any convenient manner.

At block 433, a machine learning model is trained to predict the presence of analytes of interest in the biological sample. Such model is trained by applying a training set consisting of tensor data structures corresponding to precursors of training analytes and can, in addition, include precursors of decoy analytes. A separate machine learning model is trained to predict levels of analytes of interest in the biological sample. Such model is trained by applying tensor data structures corresponding to precursors of training analytes and can, in addition, include precursors of decoy analytes. In the event weak predictions were obtained in blocks 431 and 432, such weak predictions are incorporated into training the model insofar as such weak predictions are associated with the relevant tensor data structures. Any convenient machine learning training technique may be applied, as such training techniques are known in the art, such as, for example, supervised learning, unsupervised learning or semi-supervised learning. In some cases, a round robin approach to training the machine learning model may be applied, as described above, wherein available tensors are divided into different partitions and such partitions are alternately, i.e., in a round robin manner, used for training or prediction.

In some cases, a model is trained to predict the presence of analytes of interest and a separate model is trained to predict levels of analytes of interest. In other cases, a first aspect of a single model is trained to predict the presence of analytes of interest and a second aspect of the same model is trained to predict levels of analytes of interest.

FIG. 4D depicts steps subsequent to block 430 of the exemplary embodiment of method for estimating the presence of, or levels of, analytes of interest 400. In FIG. 4D, the method proceeds to block 440, which comprises using a machine learning model to predict the presence of analytes of interest in a biological sample, where the machine learning model was trained in block 430, and/or to estimate levels of analytes of interest in a biological sample, where the machine learning model was trained in block 430. In embodiments, the model may be applied to make predictions regarding biological samples that differ from the biological sample(s) used to train the model (i.e., a different biological sample than that collected in block 412 of FIG. 4A).

At block 441, a trained machine learning model is used to predict the presence of precursors of analytes of interest by applying the machine learning model to tensors corresponding to precursors of analytes of interest (i.e., tensors that hold excerpts of preprocessed LC-MS/MS data derived from the biological sample in block 417). Predictions about the presence of precursors of analytes of interest may comprise a binary indication of whether the precursor is present or not and, in some cases, may be or may include a likelihood or confidence score indicating the likelihood or confidence that a precursor of an analyte of interest is present in the biological sample.

Also at block 441, a trained machine learning model is used to estimate levels of precursors of analytes of interest by applying the machine learning model to tensors corresponding to precursors of analytes of interest. Estimates of the levels of precursors of analytes of interest may comprise a value indicating the level of the precursor (in absolute terms or relative to other precursors) and, in some cases, may include a likelihood or confidence score indicating the likelihood or confidence that the estimated level of precursor of an analyte of interest is in the biological sample is accurately estimated by the model, or give a range of likely values.

At block 442, predictions about the presence of analytes of interest are obtained based on predictions of the presence of precursors of analytes of interest and estimates of levels of analytes of interest are obtained based on estimates of levels of precursors of analytes of interest. As described in detail above, precursors are components or parts of analytes of interest such that the presence of, or levels of, a precursor of an analyte of interest is indicative of the presence of, or level of, the corresponding analyte of interest. Any convenient technique for interpreting the presence of, or the levels of, precursors of analytes of interest may be used to obtain prediction of the presence of or the levels of corresponding analytes of interest.

### Exemplary Embodiment—Creating a Tensor Data Structure

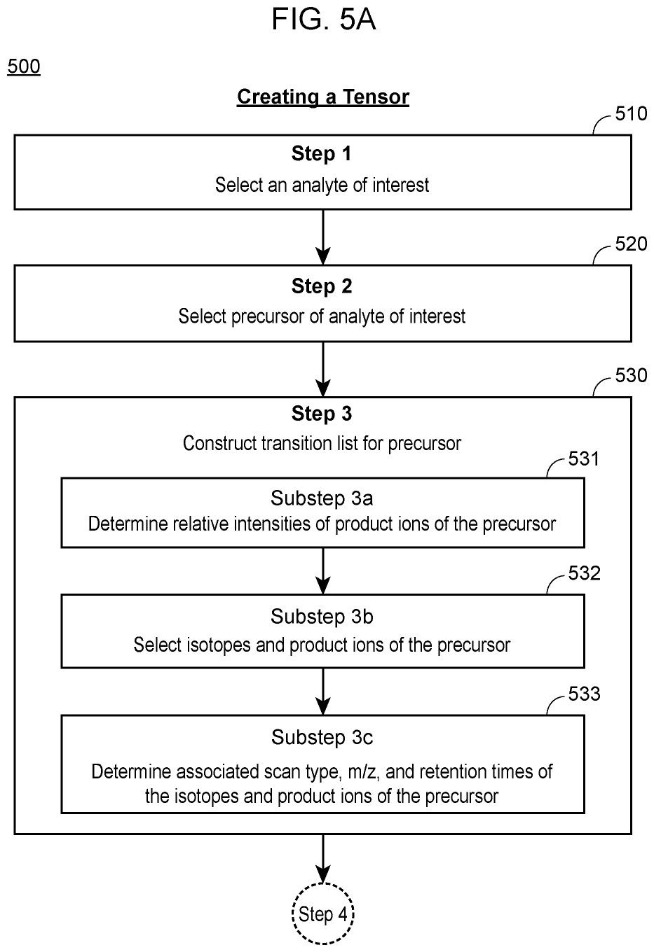

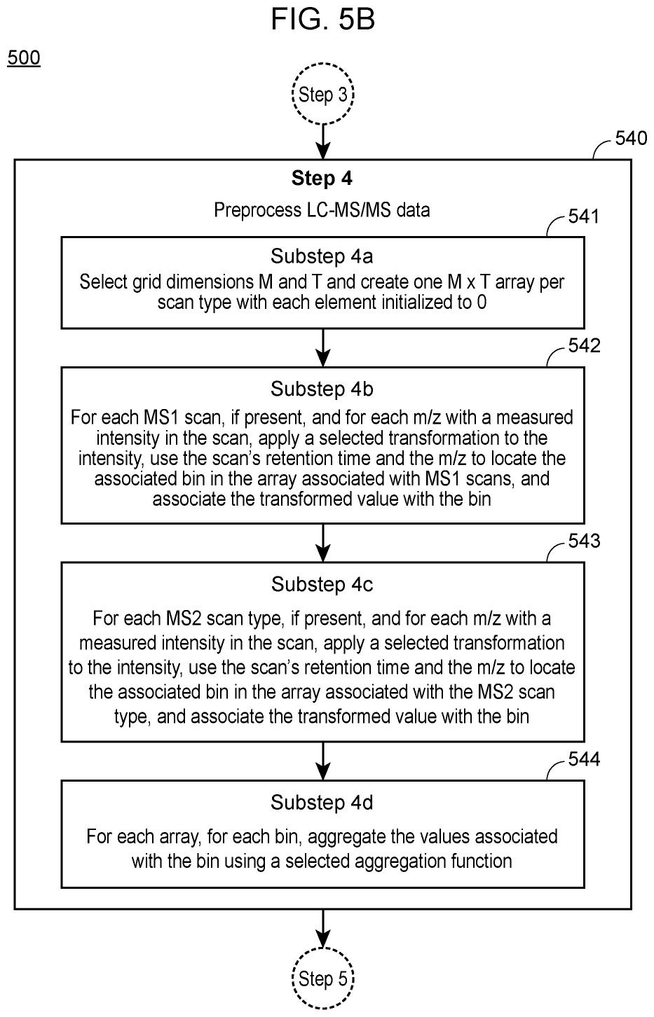

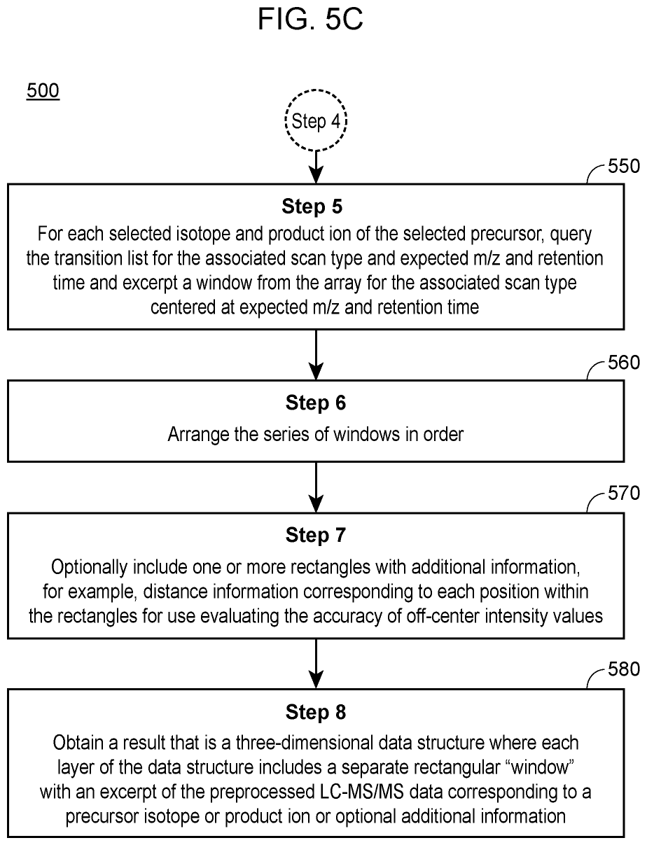

FIGS. 5A, 5B and 5C illustrate a flow diagram for creating a tensor data structure according to some aspects of the present disclosure. The tensor data structure created in exemplary embodiment of method for creating a tensor 500 comprises preprocessed LC-MS/MS data for a precursor of an analyte of interest or decoy analyte, as the case may be. FIG. 5A depicts aspects of flow diagram for creating a tensor 500.

At block 510 of embodiment for creating a tensor 500, analytes of interest (or training analytes or decoy analytes, as the case may be) are identified. Analytes of interest may be identified in any convenient manner, such as identified based on their demonstrated or potential relationship with a suspected condition or characteristic of a known condition or their demonstrated or potential relationship with an indicated treatment or they may be included on a discovery basis for example including all known or predicted proteins in the human proteome. Analytes of interest may or may not be present in a biological sample collected from a subject and, if they are present, may be present at unknown levels. Embodiments of methods according to the present invention may be used to obtain estimates about the presence of, and levels of, analytes of interest, in part by using tensor data structures such as tensors created according to the exemplary embodiment of method for creating a tensor 500.

At block 520, a precursor of the analyte of interest (i.e., from block 510) is identified. While block 520 depicts a single precursor associated with an analyte of interest, in general, one or more precursors may be created from a single analyte of interest (or training analyte or decoy analyte, as the case may be). As described in detail above, a precursor is a charged sub-part or constituent component of the analyte of interest and may be identified in any convenient manner, such as, for example, via laboratory analysis or based on results of previously conducted experiments or based on application of a computational model or based on looking up reference information in libraries or databases of precursors of analytes of interest available and known in the art. A single analyte can produce one or more precursors and different precursors can represent the same constituent component of the analyte but have different charges.

At block 530, a transition list for the precursor is constructed. At blocks 531, 532 and 533, relative intensities of product ions of the precursor of the analyte of interest are determined and isotopes and product ions of the precursor of the analyte of interest are selected and various data about the isotopes and product ions are obtained. Relative intensities of product ions (block 531) may be determined in any convenient manner, such as, for example, via laboratory analysis including LC-MS/MS or based on results of previously conducted experiments or based on application of a computational model or based on looking up reference information in libraries or databases of precursors of analytes of interest available and known in the art. This and other information may be used to select which isotopes and product ions of the precursor to include in the tensor and in which order (block 532). For example, the M, M+1, and M+2 isotopes may be the first three components to include, followed by, for example, the six most intense product ions ordered from highest to lowest relative intensity.

At block 533, expected liquid chromatographic retention time information and expected mass-to-charge ratio information is identified for each isotope and product ion of the precursor. Scan type information may also be collected at block 533. Retention time information and expected mass-to-charge ratio may be predicted based on laboratory analysis of the biological sample or based on results of previous laboratory analysis or based on looking up reference information about retention times or based on computational analysis or combinations thereof. Retention time information and expected mass-to-charge ratio may be relevant for locating corresponding intensity data in preprocessed LC-MS/MS data results, such as preprocessed SWATH data results, which may be organized or indexed based on SWATH window index, retention time, and mass-to-charge ratio. In the case of preprocessed LC-MS/MS data, retention time information is expected to be identical for each isotope and product ion since a liquid chromatography technique is typically applied prior to a mass spectrometry technique and breaking a precursor into product ions is typically caused by the mass spectrometry technique that follows the liquid chromatography technique. In the case of SWATH LC-MS/MS approaches the SWATH window index for a precursor is also determined for retrieval of intensity information related to product ions from the appropriate mass spectrometry scans.

FIG. 5B depicts steps subsequent to block 530 of the exemplary embodiment for creating a tensor 500. In FIG. 5B, the method proceeds to block 540, which comprises preprocessing the raw LC-MS/MS data to facilitate creation of windows. LC-MS/MS data can be thought of as a two-dimensional or set of two-dimensional images with one dimension indexed by mass-to-charge ratio and the other dimension indexed by retention time or scan index. Each scan is associated with a retention time by the LC-MS/MS instrumentation and software. For LC-MS data with only MS1 scans, each sample data can be represented by a single image. For SWATH LC-MS/MS data with N MS2 windows and MS1 measurements there are N+1 images, where one image corresponds to the aggregation of MS1 scans and each of the other N images composed of the scans corresponding to a SWATH window. Other approaches to LC-MS/MS can be considered in a similar fashion. The values in the image correspond to observed intensities at measured m/z in each scan. Scans corresponding to MS1 are aggregated and similarly for each of the N MS2 windows if present. An M×T grid is selected composed of M possibly overlapping ranges of m/z values as well as T possibly overlapping ranges of retention time values. For each MS2 window if measured and the MS1 measurement if measured an M×T tensor is initialized with values of 0 in each element. For each observed m/z value in each scan of the MS1 scans, if present, the grid location or locations in the associated tensor is determined and the measured intensity is associated with that location. This is similarly done for each of the MS2 windows if present. Finally, all intensities associated with a particular grid location in each tensor are aggregated in some way, which could include taking an average, the median value, the maximum value, or some other calculation. Transformations of values may be applied before or after aggregation and can include applying logarithm or tangent functions or other functions that are known in the art. At block 541, grid dimensions M and T are selected and a M×T array per scan type is created with each element initialized to 0. At block 542, for each MS1 scan, if present, and for each m/z with a measured intensity in the scan, a selected transformation to the intensity is applied, the scan's retention time and the m/z are used to locate the associated bin in the array associated with MS1 scans, and the transformed value is associated with the bin. At block 543, for each MS2 scan type, if present, and for each m/z with a measured intensity in the scan, a selected transformation to the intensity is applied, the scan's retention time and the m/z are used to locate the associated bin in the array associated with the MS2 scan type, and the transformed value is associated with the bin. At block 544, for each array, for each bin, the values associated with the bin are aggregated using a selected aggregation function and a selected transformation to the aggregated intensity is applied.

FIG. 5C depicts steps subsequent to block 540 of the exemplary embodiment for creating a tensor 500. At block 550, a rectangular window of the LC-MS/MS data is excerpted for each isotope and product ion of the precursor from the preprocessed image from block 540 appropriate to that isotope or product ion. For example, windows corresponding to isotopes are extracted from the preprocessed image containing data from the MS1 scans, while a product ion's window is extracted from the preprocessed image corresponding to the SWATH window for the product's precursor. One axis of each rectangle corresponds to different retention times and the other axis to different mass-to-charge ratios. The dimensions of the rectangular window may be any convenient length or width (corresponding to any convenient retention time and mass-to-charge ratio) and may vary. In general, the rectangular excerpts are configured so that the center of each rectangular excerpt corresponds to the expected retention time and expected mass-to-charge ratio of the corresponding isotope or product ion of the precursor. Excerpting rectangles of preprocessed LC-MS/MS data for each isotope and product ion of the precursor results in a collection of excerpts of the LC-MS/MS data showing the intensity data (i.e., corresponding to the presence and quantity) for analytes present in those rectangles.

At block 560, the plurality of rectangular excerpts of LC-MS/MS data collected at block 550 are arranged in an ordered series of rectangles. Any convenient order may be applied, such as excerpts corresponding to precursor isotopes followed by excerpts corresponding to product ions, in each case ordered according to expected intensities, for example. Since each rectangular excerpt is centered around the expected retention time and mass-to-charge ratio of the isotopes and fragment ions, intensity data at the center of each rectangle is expected to correspond to the presence of or the level of the precursor, from which the isotopes and product ions are derived.

At block 570, one or more rectangles with additional information are optionally provided for inclusion in the ordered series of rectangles generated at block 560. Example optional rectangles may include rectangles with information about the distance of each position within the rectangle from the center of the rectangle. Rectangles that indicate distances from center may relate to distance from center in the retention time dimension or in the mass-to-charge dimension. Since intensity data for each isotope and fragment ion is expected to be maximal at the center of each rectangle, rectangles that indicate distances from center may benefit training and applying a model that processes tensor data structures since such additional rectangles, and the data they contain, indicate to the model an extent to which off-center intensity data might be discounted or disregarded.

At block 580, the resulting tensor data structure is obtained comprising the excerpted rectangles of LC-MS/MS data generated at block 550 and ordered at block 560 as well as the optional distance information generated at block 570. The resulting tensor data structure comprises mass spectrometric intensity data corresponding to ions with similar mass-to-charge ratio and retention time to those expected of the collection of isotopes and product ions of the precursor selected at block 520. Moreover, the rectangular excerpts are centered such that “looking down” the center of the collection of excerpted rectangles lines up the intensity data for each isotope and product ion (i.e., relating to the presence of, or levels of, each isotope or fragment ion). Since each product ion and isotope are associated with a precursor, the presence of and/or the levels of each isotope and product ion are associated with the presence of and/or the levels of the corresponding precursor.

### Exemplary Embodiment—Applying an LC-MS/MS Analysis Technique

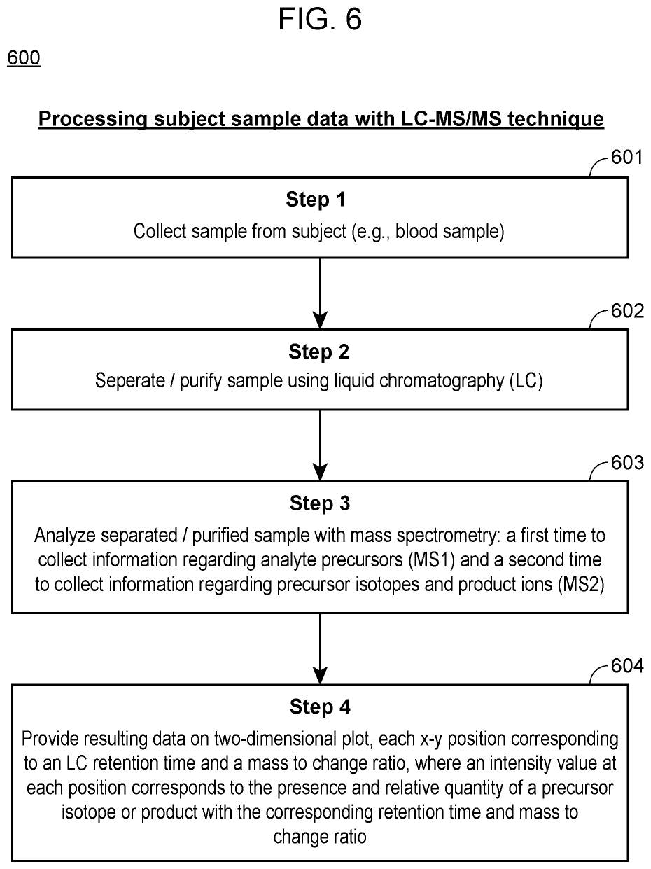

FIG. 6 illustrates a flow diagram for processing a biological sample from a subject using an LC-MS/MS technique according to some aspects of the present disclosure 600.

At block 601, a biological sample is collected from a subject, as such is explained in connection with collecting samples in FIGS. 1-4. At block 602, the sample is separated based on a physical property of the components of the sample using liquid chromatography, as such techniques are known and practiced in the art. At block 603, the separated sample is further analyzed with mass spectrometry, where precursor ions and, optionally, product ions are analyzed using MS1-only, DIA, or DDA approaches, as such approaches are known in the art. Resulting data comprises MS1 and, optionally, MS2 scans, each scan with an associated retention time, which are comprised of intensities at different mass-to-charge ratios.

At block 604, the raw LC-MS or LC-MS/MS data is preprocessed into one or more two-dimensional images where each x-y position within an image corresponds to a liquid chromatographic retention time and a mass-to-charge ratio and where an intensity value at each x-y position corresponds to the presence and relative quantities of ions with retention time and mass-to-charge ratio corresponding to the position of the intensity data in the two-dimensional image.

### Exemplary Embodiment—Transition List

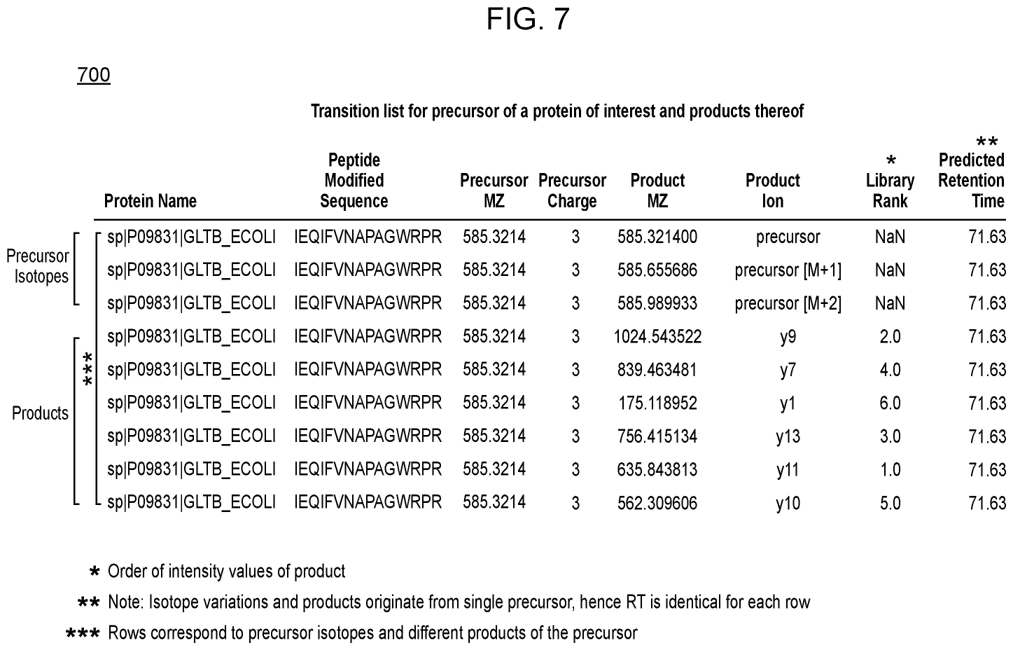

FIG. 7 provides an exemplary transition list 700 for a precursor of an analyte of interest, where the analyte of interest is a protein and the precursor is a charged peptide that is a subset or excerpt of the chain of amino acids that comprise the protein. A transition list refers to a table of the collection of isotopes and product ions that are analyzed and for which LC-MS/MS data is obtained. As seen in transition list 700, each row of the table represents a different isotope or product ion of the precursor as well as analytical information about the isotope or product ion, such as the predicted retention time and mass-to-charge ratio. The transition list is ordered according to any convenient pattern, such as shown in transition list 700 where precursor isotopes are followed by product ions. The order of the isotopes and product ions in the transition list may be used to order the excerpts of preprocessed LC-MS/MS data in the tensor data structure (i.e., as described in connection with block 560 of FIG. 5C).

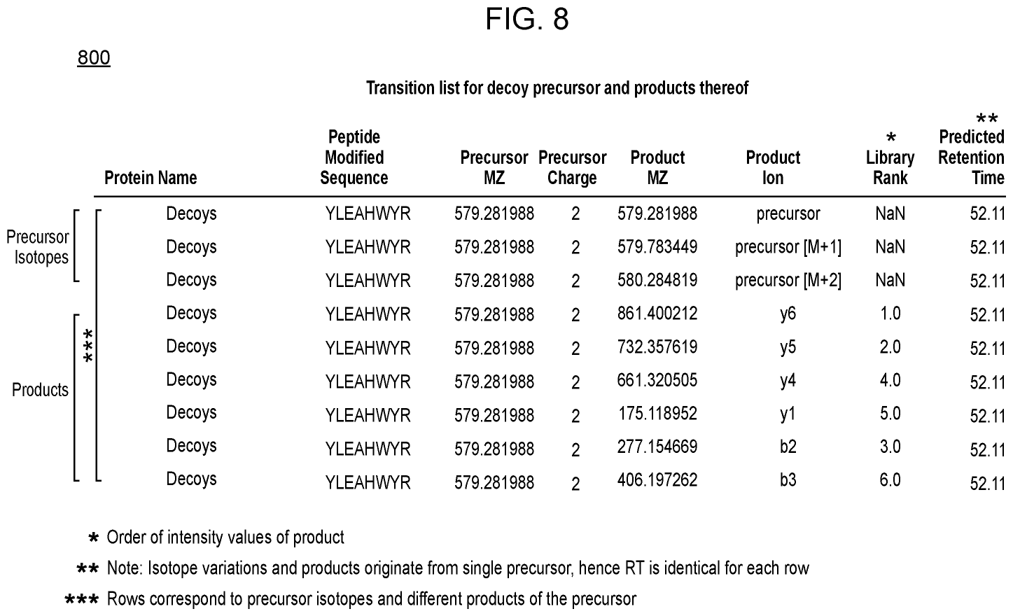

FIG. 8 presents a transition list 800 of a decoy analyte. Transition list 800 is structured in an identical manner as transition list 700 seen in FIG. 7 but that the precursor corresponding to the isotopes and product ions listed in transition list 800 are expected to be not present in the biological sample under investigation whereas the precursor of transition list 700 may or may not be present in the biological sample under investigation.

### Exemplary Embodiment—Tensor Data Structures

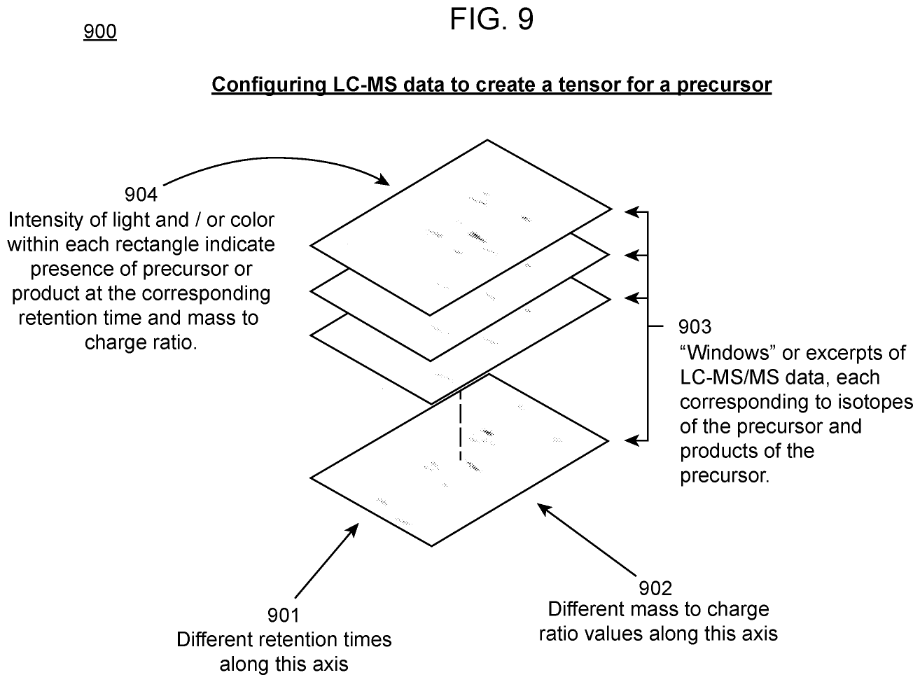

FIG. 9 depicts an exemplary tensor data structure 900 corresponding to a precursor. A collection of excerpted rectangles, i.e., “windows,” 903 of preprocessed LC-MS/MS data that include intensity data centered on the expected retention times and mass-to-charge ratios for isotopes and product ions of the precursor are depicted as an array of stacked rectangles, which, together, form a three-dimensional array of preprocessed LC-MS/MS data. The center of each rectangular window corresponds to the expected retention time and mass-to-charge ratio of each isotope and product ion such that intensity data in the center of each rectangle is expected to correspond with the presence of, or level of, the precursor associated with the isotopes and product ions.

Each rectangle is a two-dimensional array where a first axis 901 corresponds to different binned liquid chromatographic retention times (or mass spectrometric scan indices) and a second axis 902 corresponds to different binned mass spectrometric mass-to-charge ratios. Intensity data 904 is shown as the darker regions in each rectangle 903. Intensity data is binned intensity from the raw mass spectrometry data and may correspond to the presence of one or more ions at that mass-to-charge ratio and retention time. In embodiments, intensity data may be reflected by different colors or different numeric values at each position of each rectangle 903. While intensity data 904 in rectangles 903 is shown in black and white, in embodiments, the intensity data may reflect a broader range of values, which range may depend on a variety of factors, such as, for example, the sensitivity of mass spectrometric analysis techniques applied to the precursor. A high intensity data at a particular position on the first and second axis of rectangle 903 indicates the presence of an isotope or product ion with the retention time and mass-to-charge ratio corresponding to such position (though such ions may correspond to analytes other than the one of interest or other precursors thereof).

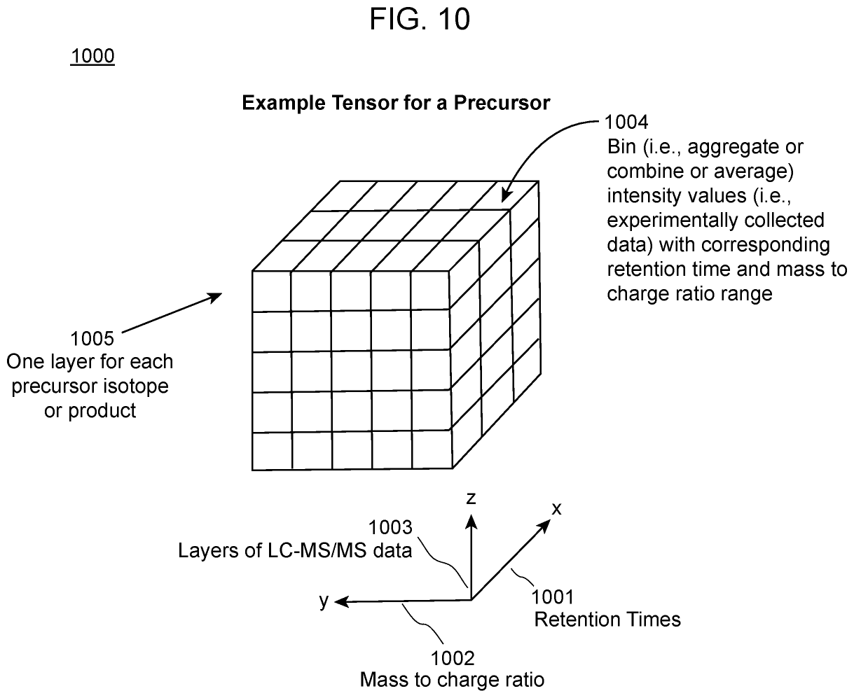

FIG. 10 depicts another exemplary tensor data structure 1000 corresponding to a precursor molecule. Tensor data structure 1000 is structured in the same way as tensor data structure 900, where rectangular excerpts of preprocessed LC-MS/MS data are stacked together in layers 1005, where each layer corresponds to a rectangular excerpt of preprocessed LC-MS/MS data. Each rectangular excerpt or layer 1005 is a two-dimensional array with a first dimension corresponding to binned retention time values 1001 and a second dimension corresponding to binned mass-to-charge ratios 1002. The collection of layers is aggregated in a stack such that third dimension 1003 corresponds to the different rectangular excerpts of preprocessed LC-MS/MS data.

In tensor data structure 1000, intensity data in each rectangular excerpt 1005 of preprocessed LC-MS/MS data is binned, i.e., aggregated into squares 1004, where each square comprises a value that may be, for example, an average intensity value over a range of retention time and mass-to-charge ratios corresponding to square 1004. The averaged intensity values may also have a function applied to them before or after aggregation such as taking a logarithmic value of the intensity or intensities. The depiction of squares 1004 comprising rectangular windows 1005 comprising a stack of rectangular windows illustrates how tensor data structure 1000 may be represented as a three-dimensional array.

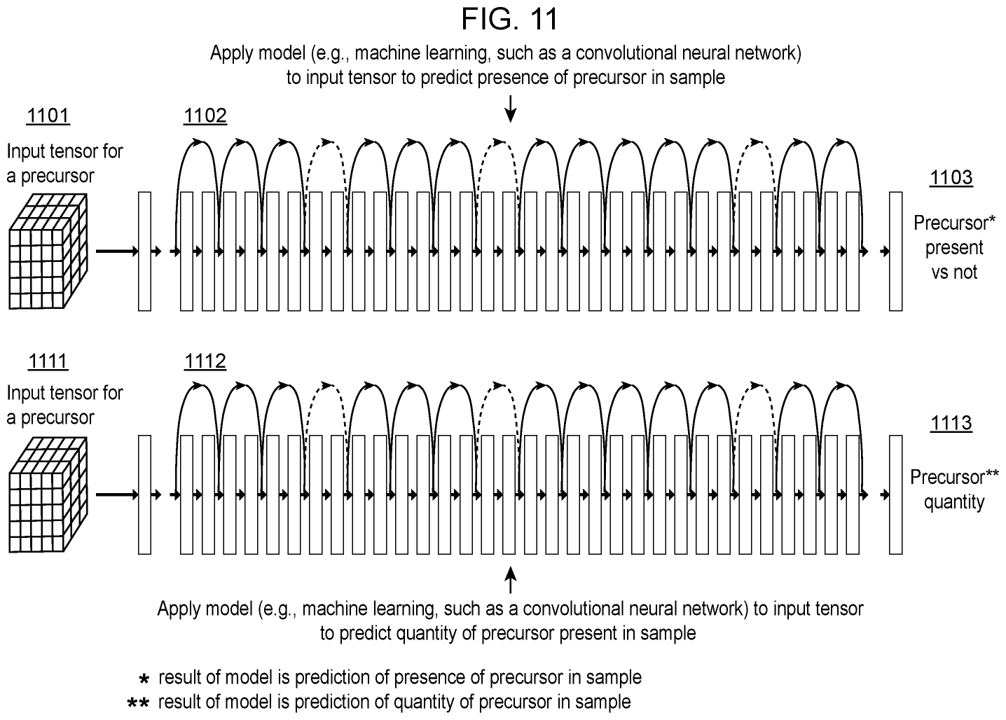

FIG. 11 depicts exemplary application of model 1102 to input tensor 1101 to predict whether the precursor represented by tensor 1101 is present in a biological sample. Input tensor 1101 is comprised of preprocessed LC-MS/MS data derived from a biological sample, as described herein. Application of model 1102 is depicted as a series of steps resulting in prediction 1103 of whether the precursor represented by tensor 1101 is present in the biological sample. Model 1102 has been trained to predict the presence of precursors and may be any convenient model known in the art, such as, for example, a machine learning model such as a convolutional neural network or a statistical model or the like.

FIG. 11 also depicts exemplary application of model 1112 to input tensor 1111 to predict a level (i.e., a quantity, such as a relative quantity) of the precursor represented by tensor 1111 present in a biological sample. Input tensor 1111 is comprised of preprocessed LC-MS/MS data derived from a biological sample. Application of model 1112 is depicted as a series of steps resulting in prediction 1113 of the level of (i.e., the quantity of, such as the relative quantity of) the precursor represented by tensor 1111 present in the biological sample. Model 1112 has been trained to predict the level of precursors and may be any convenient model known in the art, such as, for example, a machine learning model such as a convolutional neural network or a statistical model or the like.

### Exemplary Embodiment—Prediction of Presence and Levels of Analytes

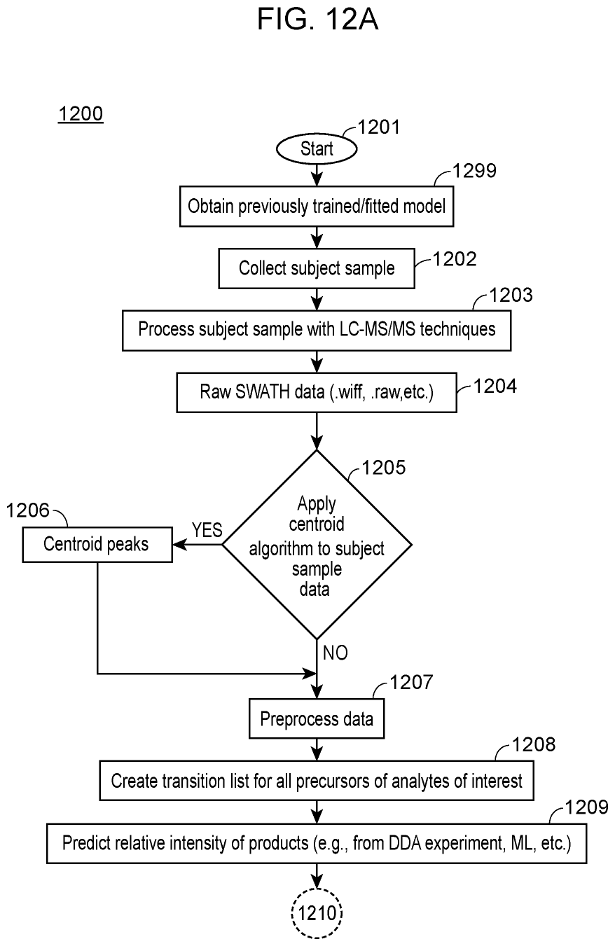

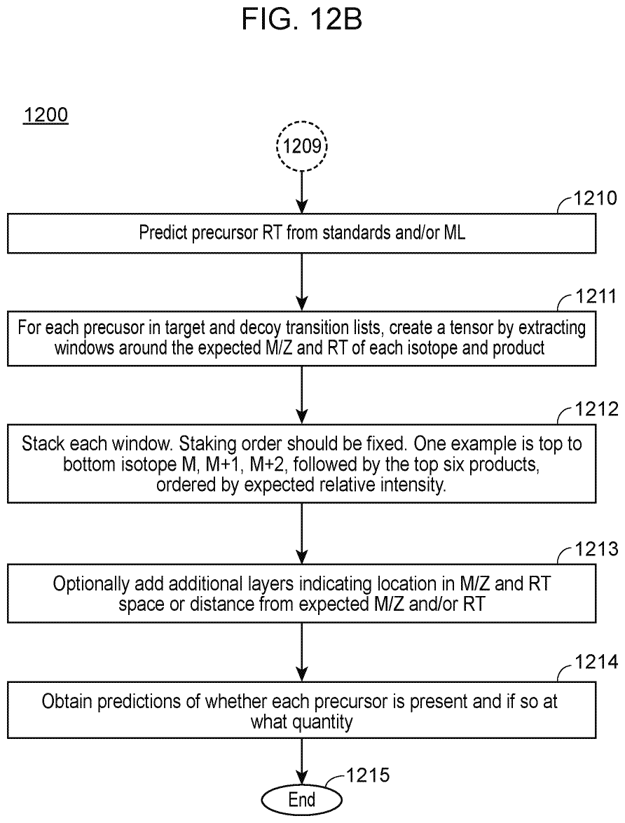

FIGS. 12A-12B depict a flow diagram for processing a biological sample from a subject with an embodiment according to some aspects of the present disclosure 1200. Additional details related to various steps set forth in process 1200 are provided below under Experimental.

FIG. 12A presents initial steps in process 1200, including block 1201, the start step where process 1200 begins. At block 1299, a previously trained or fitted model is obtained.

At block 1202, a biological sample is collected, such as a sample from a subject, as described herein, including in connection with block 102 of FIG. 1, block 203 of FIG. 2, block 303 of FIG. 3, block 412 of FIG. 4A and block 601 of FIG. 6. At block 1203, the sample collected at block 1202 is analyzed using LC-MS/MS analysis techniques to obtain LC-MS/MS data regarding the biological sample. The LC-MS/MS data collection and analysis technique may be configured to generate raw SWATH data as depicted in block 1204. Raw SWATH data is described above and is known in the art, including as described in, for example, C. Ludwig, et al., Data-independent acquisition-based SWATH-MS for quantitative proteomics: a tutorial, Mol Syst Biol. 2018 August; 14(8): e8126 (doi: 10.15252/msb.20178126), incorporated herein by reference. SWATH data in block 1204 may be held in files configured as .wiff or .raw data files, as such file types are known in the art.

At block 1205, the raw file is converted to a raw file in an open source format such as .mzML or .mzXML and a determination is made as to whether or not to apply a centroid algorithm to the raw SWATH data. The centroid algorithm may be applied based on any convenient factor, such as, for example, available processing resources or a determination of the likelihood that applying a centroid algorithm will improve predictions resulting from applying embodiment 1200. In the event a centroid algorithm is applied, the process moves to block 1206 next, at which point any convenient centroid algorithm, known in the art and as described further below, may be applied to the raw LC-MS/MS data.

At block 1207, intensity data measured by applying LC-MS/MS analysis is preprocessed and binned, i.e., aggregated into buckets in one or more arrays with buckets corresponding to a range of mass-to-charge values and a range of retention time values and with functions applied to transform the intensities either before aggregation or after. Binning at block 1207 proceeds in the same way whether or not a centroid algorithm was applied to the raw data at block 1206 or not. Binning and array creation occurs separately for MS1 scans and for scans associated with distinct components of MS2, if present, for example scans associated with each window in a SWATH analysis.

At block 1208, transition lists are created for each precursor of analytes of interest. Transition lists are developed based on the LC-MS/MS data collected as well as reference data or computational analysis or results of previous analysis techniques. Each transition list created at block 1208 corresponds to a single precursor and may take the form of exemplary transition list 700 of FIG. 7.

At block 1209, predictions are made about relative intensities of product ions and isotopes that comprise each precursor transition list and each decoy analyte transition list and are subsequently added as a column in the transition list. Predictions may be made in any convenient manner known in the art, such as from laboratory experiment applying data dependent acquisition (DDA), as such are known in the art, or training a model, such as a machine learning model, such as a deep learning model, to predict relative intensities of isotopes and product ions.

FIG. 12B presents subsequent steps in process 1200 where process 1200 continues after block 1209. At block 1210, liquid chromatographic retention times for each precursor is predicted. Retention times may be predicted using the collected LC-MS/MS data, for example by using iRT peptides or processing with a tool such as Skyline or DIA-NN or some combination of techniques, as such are known in the art and described herein. Retention times may also be predicted based on previously collected data, such as standard data or online libraries or databases making available the results of other experiments. In other cases, retention times may be predicted by training a model, such as a machine learning model, such as a deep learning model, to predict retention times of precursors.

At block 1211, an initial step of creating a tensor data structure is taken for each precursor of an analyte of interest and decoy analyte corresponding to the transition lists created at block 1208. Tensor data structures may be created, for example, in the manner set forth in FIGS. 5A-5C and the accompanying disclosure. An initial step of creating a tensor data structure at block 1211 includes extracting windows (i.e., rectangular excerpts) of preprocessed LC-MS/MS data around the expected mass-to-charge ration (M/Z) and retention time (RT) of each isotope and product of each precursor of an analyte of interest or decoy analyte.

At block 1212, each window (i.e., rectangular excerpts) of preprocessed LC-MS/MS data is stacked on top of one another, creating a three-dimensional data structure of the windows of preprocessed LC-MS/MS data forming a tensor data structure. The stacking order of windows in each tensor should be fixed across all tensor data structures. An exemplary stacking order may be an order where top to bottom isotopes are in increasing order of mass (i.e., M, M+1, M+2), followed by the top six product ions, ordered by expected relative intensities. The stacking of windows may be processed as shown in tensor 900 in FIG. 9 or as tensor 1000 in FIG. 10.

At block 1213, additional “layers” of the tensor data structure may optionally be added. By layers, it is meant a two-dimensional array corresponding in dimensions to each rectangular excerpt of the preprocessed LC-MS/MS data. The optional layers may be configured to indicate locations in a space consisting of retention time and mass-to-charge ratio dimensions or may be configured to indicate distances from expected retention time or mass-to-charge ratios for a precursor or isotopes and product ions thereof.

At block 1214, the trained machine learning model obtained at block 1299 is applied to obtain predictions of whether each precursor is present in the biological sample collected at block 1202 and if so at what quantity. Predictions about the presence of, or levels of, precursors may themselves be indicative of, or may be combined in any convenient manner to obtain, predictions about whether each analyte of interest is present in the biological sample collected at block 1202 and if so at what quantity. Process 1200 ends at block 1215.

### Exemplary Embodiment—Estimating Characteristics of a Sample Directly

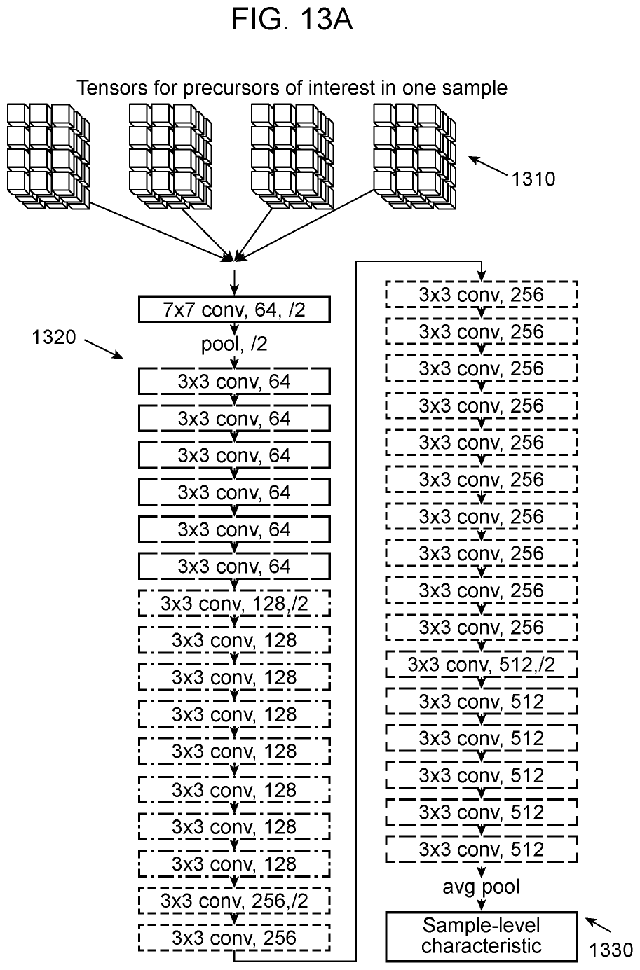

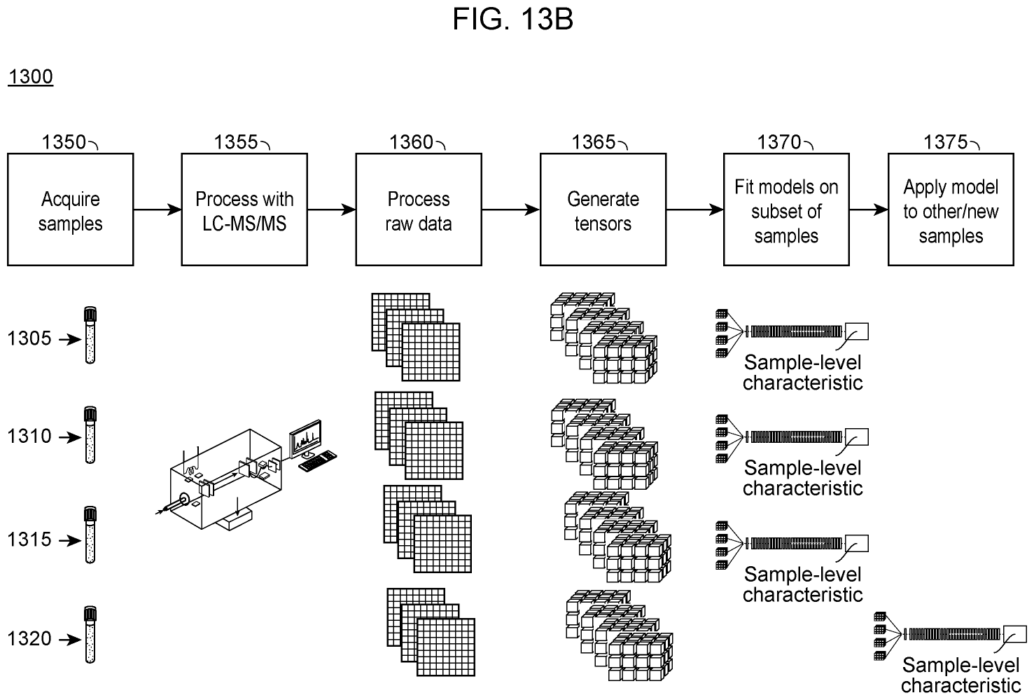

FIG. 13A depicts a diagram of using a model 1320 to estimate sample-level characteristics 1330 of a biological sample directly using inputs comprising precursors 1310 corresponding to the biological sample according to some aspects of the disclosure. That is, as described above, the model 1320 is configured to estimate characteristics of a sample 1330 without generating predictions regarding individual analytes of interest. Model 1310 has been trained on at least one biological sample that exhibits a condition and at least one biological sample that does not exhibit the condition. Applying model 1320 to input precursors 1310 produces estimates of sample level characteristics 1330, such as, for example, a degree of inflammation.

FIG. 13B depicts procedure 1300 for training and applying a model to estimate sample-level characteristics of a biological sample directly using inputs comprising precursors corresponding to biological samples according to some aspects of the disclosure. Steps 1 through 6 1350, 1355, 1360, 1365, 1370 and 1375 of procured 1300 are set out horizontally in FIG. 13B and are applied to processes/biological samples 1 through 4 1305, 1310, 1315 and 1320 set out vertically in FIG. 13B. At step 1 1350 of procedure 1300 shown in FIG. 13B, four biological samples 1305, 1310, 1315 and 1320 are acquired; at step 2 1355 of procedure 1300, biological samples 1305, 1310, 1315 and 1320 are processed using LC-MS/MS laboratory techniques to produce raw data; at step 3 1360 of procedure 1300, raw data is processed into one or more arrays, wherein the arrays may be indexed based on mass-to-charge ratio and retention time; at step 4 1365 of procedure 1300, raw data is used to generate tensor data structures, such as those described above; at step 5 1370 of procedure 1300, results of obtaining sample-level characteristics of a subset of (i.e., the first three) biological samples 1305, 1310 and 1315 are used to train (i.e., fit) a model; and at step 6 1375 of procedure 1300, the trained model is applied to fourth biological sample 1320 to predict sample level characteristics of biological sample 1320.

### Computer Implemented Embodiments

The various method and algorithm steps described in connection with the embodiments disclosed herein can be implemented as electronic hardware, computer software, or combinations of both. To clearly illustrate this interchangeability of hardware and software, various illustrative steps have been described above generally in terms of their functionality. Whether such functionality is implemented as hardware or software depends upon the particular application and design constraints imposed on the overall system applying a method according to the present disclosure. The described functionality can be implemented in varying ways for each particular application, but such implementation decisions should not be interpreted as causing a departure from the scope of the disclosure.

The various illustrative steps, components, and computing systems (such as devices, databases, interfaces, and engines) described in connection with the embodiments disclosed herein can be implemented or performed by a machine, such as a general purpose processor, a graphics processor unit (GPU) or other hardware supporting parallel processing operations, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor can also include primarily analog components. A computing environment can include any type of computer system, including, but not limited to, a computer system based on a microprocessor, a graphics processor unit, a mainframe computer, a digital signal processor, a portable computing device, a personal organizer, a device controller, and a computational engine within an appliance, to name a few.

The steps of a method, process, or algorithm described in connection with the embodiments disclosed herein can be embodied directly in hardware, in a software module executed by a processor, or in a combination of the two. A software module, engine, and associated databases can reside in memory resources such as in RAM memory, FRAM memory, GPU memory, flash memory, ROM memory, EPROM memory, EEPROM memory, registers, hard disk, a removable disk, a CD-ROM, or any other form of non-transitory computer-readable storage medium, media, or physical computer storage known in the art. An exemplary storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

The various method and algorithm steps described in connection with the embodiments disclosed herein can be implemented on any liquid chromatography and mass spectrometry software and hardware capable of generating retention time and mass spectrum data associated with precursors of analytes of interest, training analytes or decoy analytes used to generate tensor data structures. Exemplary liquid chromatography and mass spectrometry are described in P. Navarro, et al., A multi-center study benchmarks software tools for label-free proteome quantification, Nat Biotechnol. 2016 November; 34(11): 1130-1136 (doi: 10.1038/nbt.3685), incorporated herein by reference.

### Systems for Computational Analysis of Biological Samples

As summarized above, aspects of the present disclosure include systems for estimating the presence of, or levels of, analytes of interest in a biological sample. Systems for estimating a presence of analytes of interest in a biological sample according to certain embodiments comprise a processor comprising memory operably coupled to the processor, wherein the memory comprises instructions stored thereon, which, when executed by the processor, cause the processor to: obtain training liquid chromatographic and mass spectrometry data corresponding to a training biological sample, select training analytes, wherein the training analytes are analytes that may be present in the training biological sample, select decoy analytes, wherein the decoy analytes are analytes that are not expected to be present in the training biological sample, selecting precursors of the training analytes as well as precursors of the decoy analytes, obtain expected mass-to-charge ratios, predicted retention times and expected relative intensities of isotopes and product ions for each precursor of the training analytes as well as for each precursor of the decoy analytes, preprocess the training liquid chromatographic and mass spectrometry data into one or more arrays, wherein the arrays may be indexed based on mass-to-charge ratio and retention time, generating a tensor data structure for each precursor of the training analytes and for each precursor of the decoy analytes, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor, and train a model using the tensors corresponding to the precursors of the training analytes and the tensors corresponding to the decoy analytes to estimate a presence of precursors corresponding to analytes. Such systems may further comprise instructions that, when executed by the processor, cause the processor to: obtain liquid chromatographic and mass spectrometry data from a biological sample, select analytes of interest, wherein the analytes of interest are analytes that may be present in the biological sample, select precursors of the analytes of interest, obtain expected mass-to-charge ratios and predicted retention times and expected relative intensities of isotopes and product ions for each precursor of the analytes of interest, preprocess the liquid chromatographic and mass spectrometry data into one or more arrays, wherein the arrays may be indexed based on mass-to-charge ratio and retention time, generate a tensor data structure for each precursor of the analytes of interest, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor, apply the model to estimate the presence in the biological sample of the precursors corresponding to the analytes of interest, and infer the presence of analytes of interest based on estimates of the presence of the precursors corresponding to the analytes of interest.

In addition, systems capable of training models and applying models to estimate levels of analytes of interest in a biological sample are provided. Certain embodiments of such systems comprise a processor comprising memory operably coupled to the processor, wherein the memory comprises instructions stored thereon, which, when executed by the processor, cause the processor to: obtain training liquid chromatographic and mass spectrometry data from a training biological sample, select training analytes, wherein the training analytes are analytes that may be present in the training biological sample, select precursors of the training analytes, obtain expected mass-to-charge ratios, predicted retention times and expected relative intensities of isotopes and product ions for each precursor of the training analytes, preprocess the training liquid chromatographic and mass spectrometry data into one or more arrays, wherein the arrays may be indexed based on mass-to-charge ratio and retention time, generate a tensor data structure for each precursor of the training analytes, wherein each tensor comprises a three-dimensional array of excerpts of the preprocessed training liquid chromatographic and mass spectrometry data comprising intensity data centered at the expected mass-to-charge ratios and the predicted retention times of the isotopes and product ions of the precursor, and train a model using the tensors corresponding to the precursors of the training analytes to estimate levels of precursors corresponding to analytes. Other embodiments of such systems comprise a processor comprising memory operably coupled to the processor, wherein the memory comprises instructions stored thereon, which, when executed by the processor, cause the processor to: obtain training liquid chromatographic and mass spectrometry data from a training biological sample, select training analytes, wherein the training analytes are analytes that may be present in the training biological sample, select decoy analytes, wherein the decoy analytes are analytes that are not expected to be present in the training biological sample, select precursors of the training analytes as well as precursors of the decoy analytes, obtain expected mass-to-charge ratios, predicted retention times and expected relative intensities of isotopes and product ions for each precursor of the training analytes as well as for each precursor of the decoy analytes, preprocess the liquid chromatographic and mass spectrometry data into one or more arrays, wherein the arrays may be indexed based on mass-to-charge ratio and retention time, generate a tensor data structure for each precursor of the training analytes and for each precursor of the decoy analytes, wherein each tensor comprises a three-dimensional array of excerpts of the preprocessed training liquid chromatographic and mass spectrometry data comprising intensity data centered around the expected mass-to-charge ratios and the predicted retention times of the isotopes and product ions of the precursor, train a model using the tensors corresponding to the precursors of the training analytes and the tensors corresponding to the decoy analytes to estimate levels of precursors corresponding to analytes. Such systems may further comprise instructions that, when executed by the processor, cause the processor to: obtain liquid chromatographic and mass spectrometry data from a biological sample, select analytes of interest, wherein the analytes of interest are analytes that may be present in the biological sample, select precursors of the analytes of interest, obtain expected mass-to-charge ratios, predicted retention times and expected relative intensities of isotopes and product ions for each precursor of the analytes of interest, preprocess the liquid chromatographic and mass spectrometry data into one or more arrays, wherein the arrays may be indexed based on mass-to-charge ratio and retention time, generate a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor, apply the model to estimate the levels in the biological sample of the precursors corresponding to the analytes of interest, and infer the levels of analytes of interest based on estimates of the levels of the precursors corresponding to the analytes of interest.

### Characterizing a Condition:

Aspects of the present disclosure include systems for characterizing a condition of a subject based on estimates of levels of analytes of interest in a biological sample. Embodiments of systems comprising a model trained to estimate levels of analytes are capable of characterizing a condition of a subject based on estimates of levels of analytes of interest in a biological sample, wherein the memory of such systems further comprises instructions stored thereon, which, when executed by the processor, cause the processor to: obtain a biological sample from a subject, select analytes of interest, wherein the analytes of interest are analytes that may be present in the biological sample and may be associated with the condition, apply a trained model of a system of an embodiment of a system described herein, to obtain estimates of levels of the analytes of interest in the biological sample, and characterize a condition of the subject based on the estimated levels of the analytes of interest.

### Identifying a Treatment:

Aspects of the present disclosure include systems for identifying a treatment for a subject based on estimates of levels of analytes of interest in a biological sample. Embodiments of systems comprising a model trained to estimate levels of analytes are capable of identifying a treatment for a subject based on estimates of levels of analytes of interest in a biological sample, wherein the memory of such systems comprises further instructions stored thereon, which, when executed by the processor, cause the processor to: obtain a biological sample from a subject, select analytes of interest, wherein the analytes of interest are analytes that may be present in the biological sample, apply the model trained according to systems described herein to obtain estimates of levels of the analytes of interest in the biological sample, and identify a treatment for the subject based on the estimated levels of the analytes of interest in the biological sample.

### Evaluating the Effectiveness of a Treatment:

Aspects of the present disclosure include systems for evaluating effectiveness of a treatment for a condition of a subject based on estimates of levels of analytes of interest in biological samples. Embodiments of systems comprising a model trained to estimate levels of analytes are capable of evaluating effectiveness of a treatment for a condition of a subject based on estimates of levels of analytes of interest in biological samples, wherein the memory of such systems comprises further instructions stored thereon, which, when executed by the processor, cause the processor to: obtain a first biological sample from a subject at a first time, select analytes of interest, wherein the analytes of interest are analytes that may be present in the first biological sample and may be associated with a condition, apply the model trained according to systems described herein to obtain estimates of the levels of the analytes of interest in the first biological sample, apply a treatment to the subject, obtain a second biological sample from the subject at a second time, apply the model trained according to systems described herein to obtain estimates of levels of the analytes of interest in the second biological sample, compare the levels of the analytes of interest in the first and second biological samples, evaluate the effectiveness of the treatment based on the comparison of the levels of the analytes of interest.

### Recurring Interventions for a Subject Suspected of Having a Condition:

Aspects of the present disclosure include systems for a recurring treatment and evaluation of a subject suspected of having a condition. Embodiments of systems comprising a model trained to estimate levels of analytes are capable of identifying treatment and evaluation on a recurring basis for a subject suspected of having a condition, wherein the memory of such systems comprises further instructions stored thereon, which, when executed by the processor, cause the processor to: (a) select analytes of interest, wherein the analytes of interest may be associated with the condition, (b) obtain a biological sample from the subject, (c) apply the model trained according to systems described herein to obtain estimates of the levels of the analytes of interest in the biological sample, (d) identify the treatment for the subject based on the estimated levels of the analytes of interest in the biological sample, (e) recommend the treatment to the subject for a specified period of time, (f) provide recurring evaluation and treatment for the subject by repeating steps (a) through (e) one or more times.

In embodiments of systems for treatment for a subject suspected of having a condition, the recurring intervention comprises a subscription service. In other embodiments, identifying the treatment for the subject comprises identifying changes to the subject's diet. In certain embodiments, recommending the treatment to the subject comprises providing food-based treatment to the subject. In still other embodiments, providing food-based treatment to the subject comprises a food subscription service. In some cases, the recurring intervention for the subject is repeated on a periodic basis, wherein the period is determined at least in part based on the estimated levels of the analytes of interest in the biological sample. In other cases, the suspected condition is irritable bowel disease or non-alcoholic steatohepatitis or Crohn's disease or Rheumatoid arthritis or cardiovascular disease.

Any convenient processor and memory may be used in embodiments of the subject systems. For example, any off the shelf, commercially available processor or memory such as those discussed in detail above, may be used. In particular, in embodiments, the processor may comprise a general purpose processor or a graphics processing unit or other processor configured to support parallel processing operations, or combinations thereof. In instances, the processor and memory are operably connected to each other. Such operable connection may take any convenient form such that instructions and data may be obtained by the processor by any convenient input technique, such as via a wired or wireless network connection, shared memory, a bus or similar communication protocol.

### Computer-Readable Storage Media for Computational Analysis of Biological Samples

Aspects of the present disclosure further include non-transitory computer-readable storage media having instructions for practicing the subject methods. Computer-readable storage media may be employed on one or more computers for complete automation or partial automation of a system for practicing methods described herein. In certain embodiments, instructions in accordance with the methods described herein can be coded onto a computer-readable medium in the form of “programming,” where the term “computer-readable medium” as used herein refers to any non-transitory storage medium that participates in providing instructions and data to a computer for execution and processing. Any suitable non-transitory storage medium may be used, such as a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to a computer. A file containing information can be “stored” on a computer-readable medium, where “storing” means recording information such that it is accessible and retrievable at a later date by a computer.

### Utility

The subject methods and systems find use in a variety of applications where it is desirable to obtain predictions of the presence of analytes of interest in a biological sample or estimates of the levels of analytes of interest in biological samples or where it is desirable to characterize a condition based on a biological sample. In some embodiments, the methods and systems described herein find use in clinical settings, such as any clinical setting where a diagnosis of a condition may be sought, or a treatment for a condition may be evaluated. In other embodiments, the methods and systems described herein find use in remote medicine settings, where diagnosis of conditions and/or evaluation of treatments for conditions may be facilitated by application of the present methods and systems, such as in telemedicine contexts. In addition, the subject methods and systems find use in improving the effectiveness, accuracy, cost, convenience and clinical application of predicting the presence of and/or estimating levels of analytes of interest in biological samples, such as biological samples obtained from a subject. In some cases, the subject methods and systems find use in cheaper and/or more effective and/or more sustainable treatments for conditions, such as food-based interventions. As a result, in some cases the subject methods and systems find use in facilitating repeated or recurring or ongoing characterization of a treatment or evaluation of treatments for a condition. In addition, the methods and systems described herein find use in discovery applications such as scientific research. Also, the methods and systems described herein can be used to discover novel disease sub-types and discover heterogeneous treatment groups.

The following is offered by way of illustration and not by way of limitation.

### EXPERIMENTAL

A detailed description of exemplary embodiments of methods according to the present invention as well as utilization thereof in connection with conducting computational analysis of biological samples is set forth below. As discussed below, methods according to the present invention were used in connection with detection of proteins. However, it should be noted that the present invention may be used in connection with detection of other analytes, such as, for example, lipids, metabolites or other analytes, and is not limited exclusively to detection of proteins.

### Methods and Materials

### Mass Spectrometry Raw Data:

In order to obtain mass spectrometry raw data for use in an embodiment of the present invention, LFQbench data files, as such are described in P. Navarro, et al., A multi-center study benchmarks software tools for label-free proteome quantification, Nat Biotechnol. 2016 November; 34(11): 1130-1136 (doi: 10.1038/nbt.3685) (“Navarro 2017”), incorporated herein by reference, were downloaded from ProteomeXchange (PXD002952), available at the World Wide Web (www) at proteomexchange.org/.

### Spectral Library Creation:

The Trans-Proteomic Pipeline (TPP) was used to create the spectral library, where TPP is described in detail at the Seattle Proteome Center, available at World Wide Web (www) at tools.proteomecenter.org/wiki/index.php?title=Software:TPP, incorporated herein by reference. However, other spectral library creation tools and settings could alternatively be used. UniProt proteomes for H. sapiens, E. coli, and S. cerevisiae were downloaded in FASTA format. All DDA .wiff files were converted to mzXML using vendor centroiding. Comet was used to search DDA files individually using the settings specified in Navarro 2017. For this search the organism-specific FASTA files were appended with proteins from the common Repository of Adventitious Proteins (cRAP, as such is described at The Global Proteome Machine, available at World Wide Web (www) at thegpm.org/crap/, incorporated herein by reference), iRT standard peptides, and reverse sequences of all of the above. Comet scores were post-processed and rescored using PeptideProphet with the settings specified in Navarro 2017. Skyline was then used to import the nine individual pep.xml output files from PeptideProphet and create a spectral library with the appropriate iRT standards selected and a cut-off score of 0.99. The iRT standard peptides used by Navarro 2017 were Biogynosys-11 (iRT-C18).

### Transition List Creation:

A FASTA file containing all proteins from the three species' (H sapiens, E. coli, and S. cerevisiae) proteomes was used in Skyline to generate a target transition list with Trypsin [KR P] digest with zero missed cleavages. Peptides were filtered so that each protein had at least two peptides and all peptides were unique. Alternatively, other approaches could be used, including not filtering at all. Precursor charges of 2, 3, or 4, ion charges of 1 or 2, and ion types of y, b, or p were selected. Product ions from ion-3 to last ion-1 were chosen. Six product ions were used. Transition lists for other species, enzymes, number of product ions, etc. could also be used.

A decoy transition list of shuffled sequences with one decoy per target was also generated using Skyline. Other methods of decoy creation could also be used.

Skyline and mProphet SWATH-DIA Analysis:

Raw SWATH-DIA .wiff files from the HYE124_TTOF5600_64var acquisitions were converted to .mzML using MSConvert with vendor peak picking for MS1 and MS2 and 32-bit precision. MSConvert is described at M. Chambers, et al., A cross-platform toolkit for mass spectrometry and proteomics, Nature Biotechnology 30, 918-920 (2012). https://doi.org/10.1038/nbt.2377, incorporated herein by reference. Other options could be chosen including omitting peak picking. These .mzML files were loaded into Skyline using “Import:Peptide Search” with default settings except for those described above and “Integrate All” selected under “Settings”. An mProphet model was automatically trained and applied. The total area of the fragment ions was used for quantitation of precursors. The mProphet Score was used for analyses involving differentiating targets from decoys.

### DDX Algorithm (an Embodiment According to the Present Invention)—Creation of Target Transition List and Decoy Transition List:

As used herein, including in the figures hereto, “DDX” or “DDX algorithm” refers to an embodiment of the methods of the present invention.

The target transition list is created from a list of all proteins of interest. Each protein is computationally broken into peptides in the same manner as the enzyme used to digest proteins in the experiment, e.g., Trypsin. Modifications and missed cleavages can also be included. Each peptide is used to create some number of precursors with charges depending on the objectives of a consumer of an embodiment of the method, such as a clinician applying an embodiment of the method in connection with a subject or a researcher doing scientific discovery or research. Each precursor is then used to create some number of isotopes for example M, M+1, and M+2 isotopes, though others can be used. Each precursor is also used to create a list of possible fragment ions, each of which can be represented by one or more charges. Each precursor isotope and fragment ion has an expected m/z that can be calculated from the sequence and charge. The fragment ions for each precursor are then sorted from highest expected intensity to the lowest expected intensity, which intensity is denoted as their Library Rank (as such is seen in FIGS. 7-8). The expected intensities can come from the analysis of an experiment (e.g., DDA spectral library creation), or from a computational model, or some combination thereof. After sorting the fragment ions for each precursor, the top N are kept and the rest are discarded for each precursor, where Nis selected by the user. Predicted retention times are also added for each precursor. These predicted retention times can come from iRT-based approaches, machine learning or computational approaches, or from other experimental approaches that may be combined with computational approaches.

Decoy transition lists can be created in a number of ways. One example is shuffling the peptide sequences and using information from the target transition list to fill in the decoy transition list.

### Generation of Initial Quantity Values for Each Precursor:

Quantity values are generated for each target precursor using any number of possible approaches. For example, this could be done by using Skyline and mProphet to identify and quantify a peak group for each precursor in the target transition list discussed above. It is assumed that these initial quantity values are less accurate than what will be produced by the models that are embodiments of the present invention described below.

### Pre-Processing Raw SWATH or SWATH-Like Data:

The raw data is converted to an open source format such as .mzML and may also be optionally centroided (as seen at blocks 1204, 1205 and 1206 of FIG. 12A). A grid is constructed in m/z (mass to charge) and RT (retention time) dimensions where grid sizes can be specified by the user. For MS1 and each SWATH window separately, scans corresponding to either MS1 or the selected SWATH window are extracted and intensities within each grid rectangle are combined or aggregated in some way, e.g. summing or averaging (i.e., binning as seen in bin 1004 of FIG. 10). Transformations such as taking the logarithm can be applied to intensities before or after aggregation. This process results in the creation of S tensors of dimension 1×M×R, where S is the number of SWATH windows (plus one if MS1 scans are present), Mis the number of grid bins in the m/z (mass to charge) dimension, and R is the number of grid bins in the RT (retention time) dimension. Other ordering of dimensions could alternatively be used as long as they are consistent across the grids.

### Create Tensor for Each Target and Decoy Precursor:

For each target and decoy precursor in the target and decoy transition lists, we create a tensor of dimension C×H×W where each dimension can be changed based on user choice but must be consistent across all precursors. These tensors can be viewed as C layers of H×W matrices stacked on top of each other (as seen in layer 1005 of FIG. 10). In an embodiment, the first layer corresponds to the M isotope of its precursor, the second to the M+1 isotope, and the third to the M+2 isotope. To fill the values of this first layer, the expected m/z (mass to charge ratio) and RT (retention time) are queried from the transition list. A window of size H×W is extracted from the preprocessed raw data tensor corresponding to the MS1 scans above, centered on the expected m/z and retention time of this ion and the values copied into this precursor's tensor in the corresponding layer. The fourth layer corresponds to the fragment ion with the highest Library Rank for this precursor, and so forth until the ninth layer which corresponds to the fragment ion with the sixth highest Library Rank. To fill these values, the SWATH window of the precursor is determined, and an H×W sized window is extracted from the preprocessed raw data layer corresponding to the scans from this SWATH window centered on the expected m/z and retention time of this fragment ion and the values copied into this precursor's tensor in the corresponding layer.

Additional layers may be added or layers could be removed. One example could be changing or increasing or decreasing the isotope ions used for layers. Another example could be changing or increasing or decreasing the fragment ions used for layers.

Other layers could be added with other types of information. For example, a layer could be added where the value in each bin is the row index or the column index of the bin, or the value could correspond to the distance from the center of the layer in either the number of columns, rows, or some combination. This supplies location and/or error information for m/z and retention time to downstream modeling and analyses. The ordering of the layers themselves can be changed so long as the chosen ordering is consistent across precursors.

### Fit Models:

The tensors generated can now be used to fit various models (as seen in block 1217 of FIG. 12B). For example, one model that can be created determines whether a target precursor is present in a sample or not. To fit this model, target precursors above from one or more samples are labeled with a 1 and decoy precursors above from one or more samples (usually the same samples) are labeled with a 0. A model is then fit that takes the tensor for a precursor and predicts the label. This can be done with any number of modeling techniques including linear models, tree-based models, deep learning models, etc. where the shape of the tensor can be changed to accommodate the input shape required by the model. For example, the tensor can be changed to a vector for input into a linear model, or it can be changed to C×2*H×2*W using an imputation approach. A criterion can also be used to select which target precursors are used for training, e.g., a confidence score could be generated from some algorithm such as Skyline and/or mProphet and target precursors with confidence scores above a certain threshold are used for training.

Another model that can be fit predicts the quantity of a precursor. This model can be fit by using only the target precursors above from one or more samples and fitting a model that takes the precursor's tensor as input and predicts the quantity value from above. Alternatively, decoys could also be incorporated when fitting such a model, where their quantity value is set to 0.

Semi-supervised or weak supervision approaches and other machine learning techniques can also be used with the above models. For example, after making initial predictions, the most confident predictions can be used as the training set in a second iteration, and this process can be repeated until a stopping criteria is reached. This can be done for the target versus decoy model, the quantitative model, or other models.

Models can be fit and applied in a variety of ways. For example, one subset of samples can be used to fit these models, which are then applied to other samples. Another approach is to divide the targets and decoys in each sample into subsets, fit a model on one group of subsets, and apply the model to the other group of subsets, and repeat this procedure until all precursors have a prediction from a model fit on data not including that precursor. This latter approach may account for sample-specific patterns better than the former approach. A combination of these two approaches could also be used.

Multi-task learning could also be used. For example, a model predicting precursor quantity and presence at the same time could be created.

### DDX SWATH-DIA Analysis:

Target and decoy transition lists were created in Skyline (i.e., such as transition list 700 in FIGS. 7 and 800 in FIG. 8). Centroided .mzML DIA files were processed by an algorithm (the “DDX” algorithm) that is an embodiment of the present invention to create a tensor for each precursor in these transition lists. A model predicting whether a precursor was a target or a decoy (Identification Model) and a model predicting the quantity estimated by Skyline with mProphet peak group selection (Quantification Model) were fit on sample labeled A1 and used to make prediction in samples labeled A2 and A3. Similarly, models were also fit on sample labeled B1 and used to make predictions in samples labeled B2 and B3.

### False Discovery Rate Estimation:

To estimate False Discovery Rate (FDR) based on thresholding the Identification Model score, the number of decoys with a score above the threshold were calculated and divided by the number of targets with a score above the threshold.

### Evaluation of Model:

One way to evaluate the Identification Model is to look at how many target precursors pass different thresholds and plot them relative to the FDRs of those thresholds. Both an approach that is an embodiment of the present invention and an approach based on the combination of Skyline and mProphet return a score that allows these quantities to be calculated.

One way to evaluate the Quantification Model is to pair samples labeled A2 and B2 as well as A3 and B3 and look at the mean absolute error (MAE) of the log ratio of the predicted quantities across precursors against the expected log ratios. This MAE can be calculated for different thresholds on Identification Model scores, and a plot of MAE versus FDR can be created as well. For the mProphet-based approach the quantity values calculated by Skyline for the peak groups selected by mProphet were used. This approach may unfairly penalize models that are better at identifying more precursors at a given FDR, however. Thus, MAE was also plotted against the top N most confident predictions for varying values of N.

Extending Analysis from Precursors to Proteins:

Protein identification and quantification are often of more interest than precursors or peptides so an evaluation using an embodiment of the present invention was extended to them. There are numerous ways to aggregate precursor results to the protein level. One common approach is to set the protein score to be the best scoring precursor or peptide of that protein. However, larger proteins can be overrepresented due to having more peptides and thus “tries” at getting a high score. Instead, we divide the proteins into groups corresponding to the number of peptides they have in the target list and estimate a null distribution of top scores for proteins of that size from the decoys. We then calculate a p-value for each protein based on its top score and this null distribution and then apply the Benjamini-Hochberg procedure to determine the number of protein discoveries at various FDRs.

### Results:

### Precursor Identification

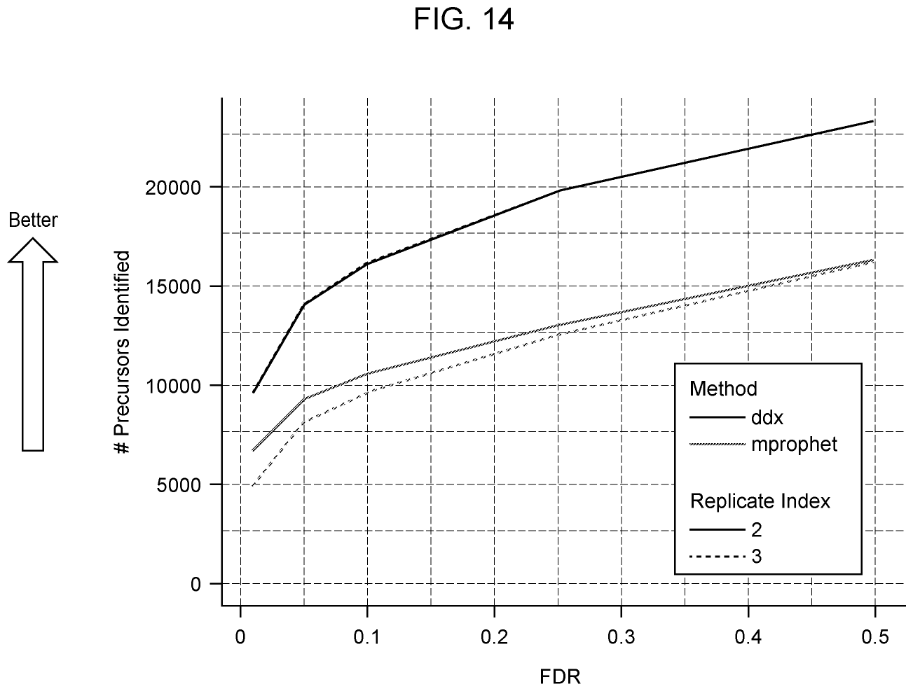

The approach according to an embodiment of the present invention (i.e., the DDX algorithm) significantly outperforms mProphet in identifying more precursors. The results of such comparison are depicted in FIG. 14. The increase was roughly 60% at a 10% FDR and was consistent across both replicate pairs.

### Precursor Quantification

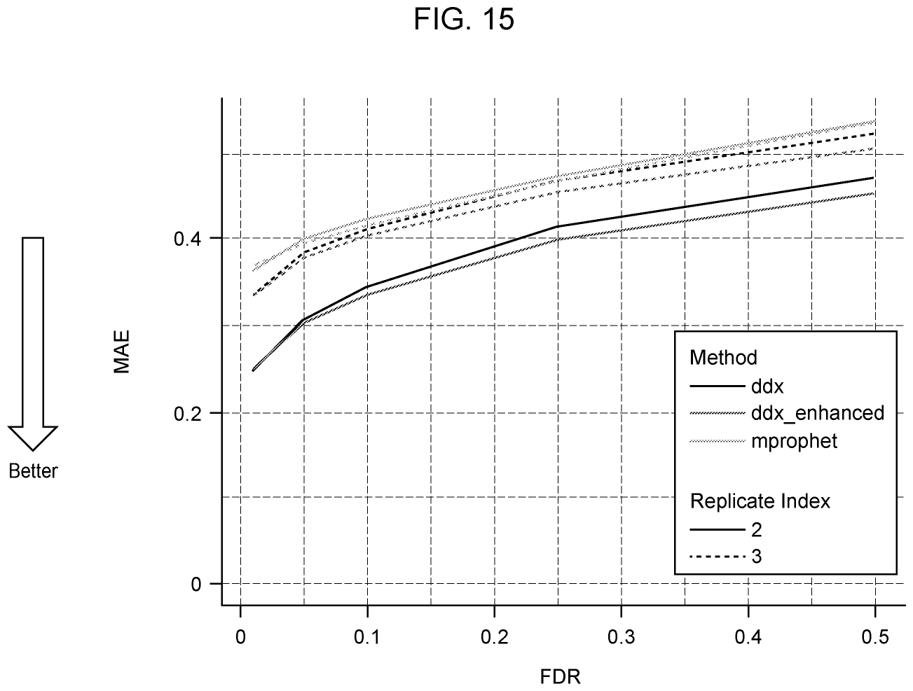

The approach according to an embodiment of the present invention (i.e., the DDX algorithm) maintains and possibly improves quantitation accuracy across the FDR range examined. The results of such comparison are depicted in FIG. 15.

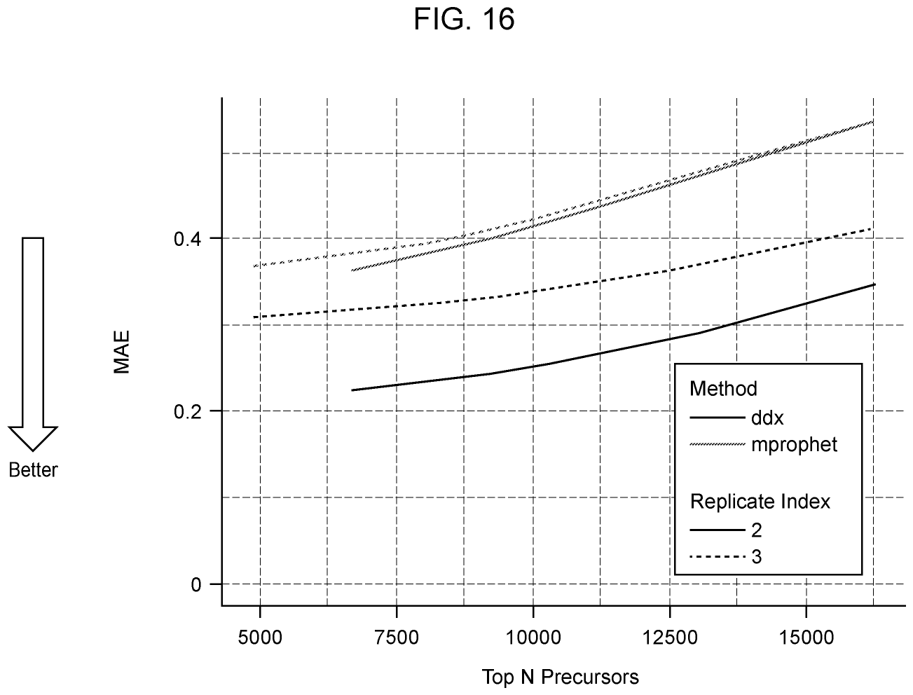

However, as mentioned above, this is not entirely a fair comparison as MAE is calculated for all precursors passing the FDR threshold and the DDX algorithm approach that is an embodiment of the present invention identifies many more than mProphet. A hypothesis was that these more difficult to identify precursors would also be more difficult to quantify, so accuracy for the top Nin each method were also compared. The results of such comparison are depicted in FIG. 16. As can be seen in FIG. 16, the DDX algorithm approach that is an embodiment of the present invention improves the quantitation accuracy in both replicate pairs.

### Protein IDENTIFICATION

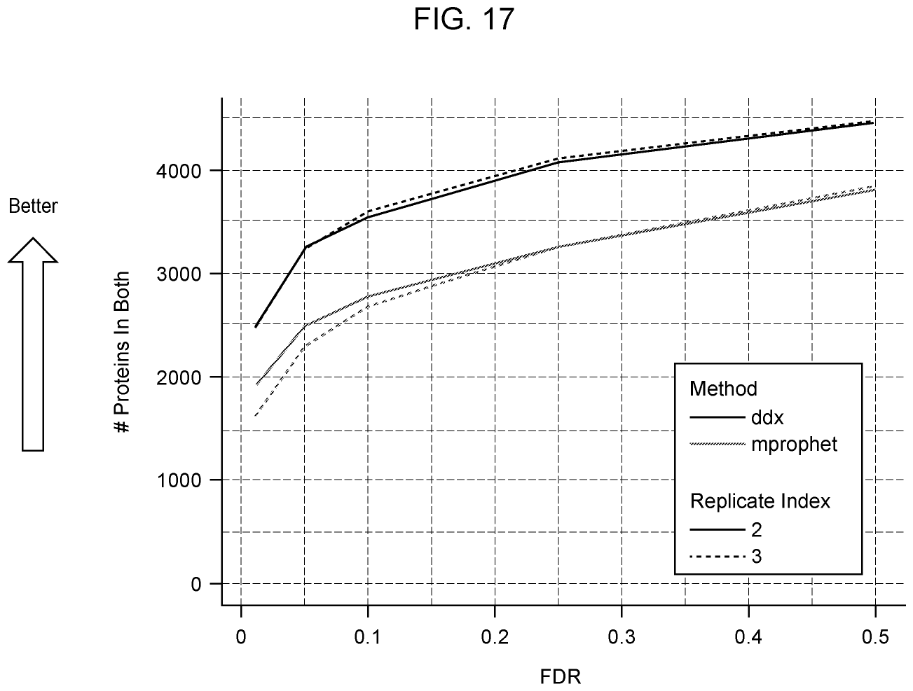

Applying the DDX algorithm approach that is an embodiment of the present invention also significantly improved protein quantification, with an approximately 30% increase at a 10% FDR. The results of such comparison are depicted in FIG. 17.

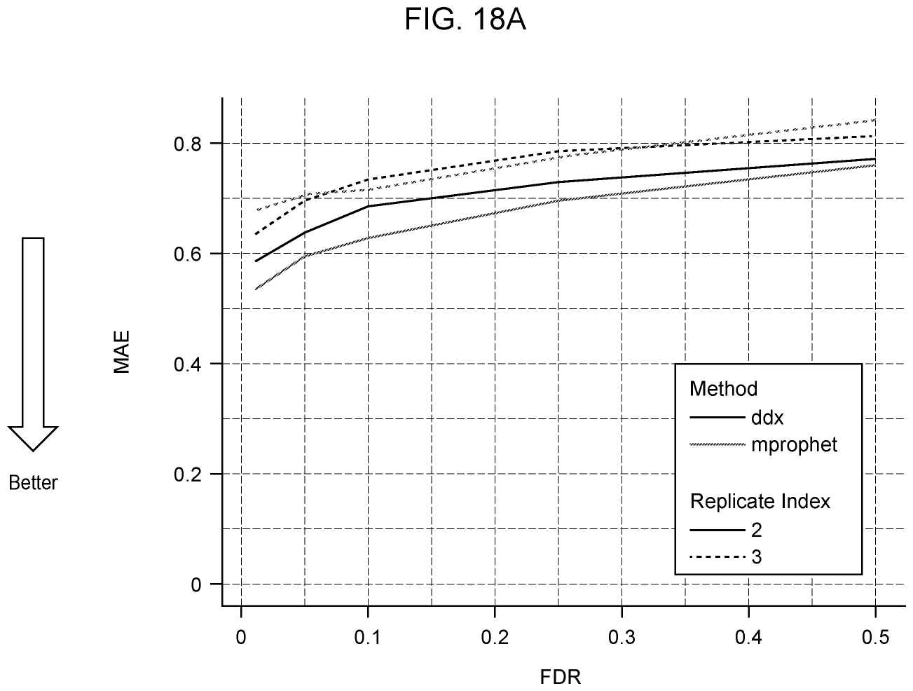

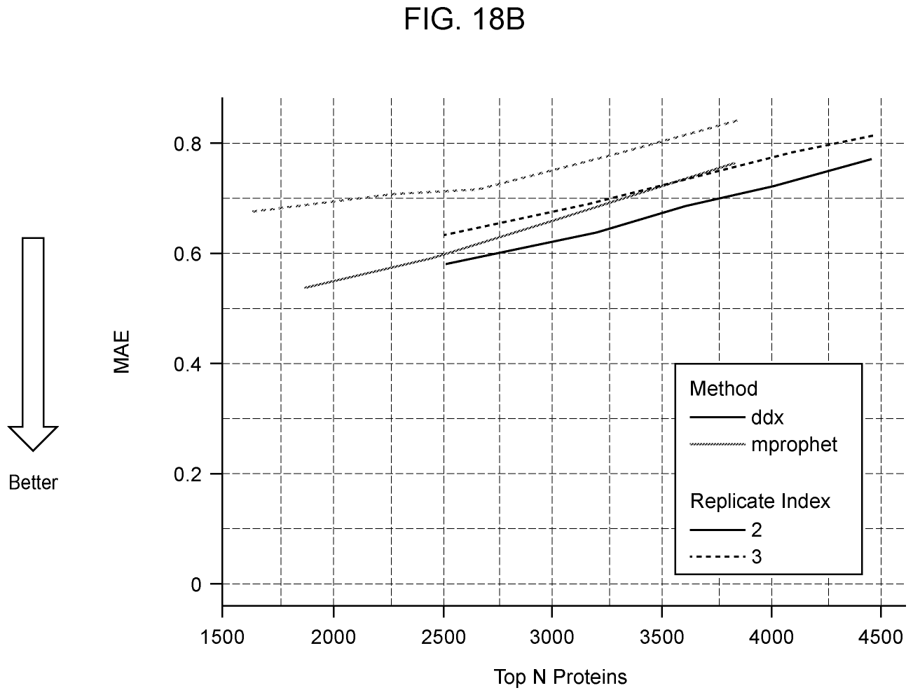

Quantitation accuracy was maintained even with this additional identification of proteins. The results of such further comparison are depicted in FIGS. 18A and 18B.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A method of training a model to estimate a presence of analytes in a biological sample, the method comprising:
obtaining liquid chromatographic and mass spectrometry data from the biological sample;
selecting training analytes, wherein the training analytes are analytes that may be present in the biological sample;
selecting decoy analytes, wherein the decoy analytes are analytes that are not expected to be present in the biological sample;
selecting precursors of the training analytes as well as precursors of the decoy analytes;
obtaining expected mass-to-charge ratios, predicted retention times and expected relative intensities of isotopes and product ions for each precursor of the training analytes as well as for each precursor of the decoy analytes;
preprocessing the liquid chromatographic and mass spectrometry data into one or more arrays, wherein the arrays may be indexed based on mass-to-charge ratio and retention time;
generating a tensor data structure for each precursor of the training analytes and for each precursor of the decoy analytes, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor; and
training a model using the tensors corresponding to the precursors of the training analytes and the tensors corresponding to the decoy analytes to estimate a presence of precursors corresponding to analytes.

2. A method of estimating a presence of analytes of interest in a biological sample, the method comprising:
obtaining liquid chromatographic and mass spectrometry data from the biological sample;
selecting the analytes of interest, wherein the analytes of interest are analytes that may be present in the biological sample;
selecting precursors of the analytes of interest;
obtaining expected mass-to-charge ratios and predicted retention times and expected relative intensities of isotopes and product ions for each precursor of the analytes of interest;
preprocessing the liquid chromatographic and mass spectrometry data into one or more arrays, wherein the arrays may be indexed based on mass-to-charge ratio and retention time;
generating a tensor data structure for each precursor of the analytes of interest, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor;
applying a model trained according to the method of claim 1 to estimate the presence in the biological sample of the precursors corresponding to the analytes of interest; and
inferring the presence of analytes of interest based on estimates of the presence of the precursors corresponding to the analytes of interest.

3. A method of training a model to estimate levels of analytes in a biological sample, the method comprising:
obtaining liquid chromatographic and mass spectrometry data from the biological sample;
selecting training analytes, wherein the training analytes are analytes that may be present in the biological sample;
selecting precursors of the training analytes;
obtaining expected mass-to-charge ratios, predicted retention times and expected relative intensities of isotopes and product ions for each precursor of the training analytes;
preprocessing the liquid chromatographic and mass spectrometry data into one or more arrays, wherein the arrays may be indexed based on mass-to-charge ratio and retention time;
generating a tensor data structure for each precursor of the training analytes, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor; and
training a model using the tensors corresponding to the precursors of the training analytes to estimate levels of precursors corresponding to analytes.

4. A method of training a model to estimate levels of analytes in a biological sample, the method comprising:
obtaining liquid chromatographic and mass spectrometry data from the biological sample;
selecting training analytes, wherein the training analytes are analytes that may be present in the biological sample;
selecting decoy analytes, wherein the decoy analytes are analytes that are not expected to be present in the biological sample;
selecting precursors of the training analytes as well as precursors of the decoy analytes;
obtaining expected mass-to-charge ratios, predicted retention times and expected relative intensities of isotopes and product ions for each precursor of the training analytes as well as for each precursor of the decoy analytes;
preprocessing the liquid chromatographic and mass spectrometry data into one or more arrays, wherein the arrays may be indexed based on mass-to-charge ratio and retention time;
generating a tensor data structure for each precursor of the training analytes and for each precursor of the decoy analytes, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor; and
training a model using the tensors corresponding to the precursors of the training analytes and the tensors corresponding to the decoy analytes to estimate levels of precursors corresponding to analytes.

5. The method of training a model to estimate levels of analytes in a biological sample according to any of claims 3 to 4, further comprising:
obtaining estimates of a presence of the training analytes in the biological sample by applying a second model trained according to the method in claim 1,
wherein training the model to estimate the levels in the biological sample of the precursors corresponding to the training analytes further comprises training the model using results of estimating the presence of the training analytes.

6. A method of estimating levels of analytes of interest in a biological sample, the method comprising:
obtaining liquid chromatographic and mass spectrometry data from the biological sample;
selecting the analytes of interest, wherein the analytes of interest are analytes that may be present in the biological sample;
selecting precursors of the analytes of interest;
obtaining expected mass-to-charge ratios, predicted retention times and expected relative intensities of isotopes and product ions for each precursor of the analytes of interest;
preprocessing the liquid chromatographic and mass spectrometry data into one or more arrays, wherein the arrays may be indexed based on mass-to-charge ratio and retention time;
generating a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor;
applying a model trained according to the method of any of claims 3 to 5 to estimate the levels in the biological sample of the precursors corresponding to the analytes of interest; and
inferring the levels of analytes of interest based on estimates of the levels of the precursors corresponding to the analytes of interest.

7. A method of characterizing a condition of a subject based on estimates of levels of analytes of interest in a biological sample, the method comprising:
obtaining a biological sample from the subject;
selecting the analytes of interest, wherein the analytes of interest are analytes that may be present in the biological sample and may be associated with the condition;
obtaining estimates of the levels of the analytes of interest in the biological sample by applying the method according to claim 6; and
characterizing the condition of the subject based on the estimated levels of the analytes of interest.

8. A method of identifying a treatment for a subject based on estimates of levels of analytes of interest in a biological sample, the method comprising:
obtaining a biological sample from the subject;
selecting the analytes of interest, wherein the analytes of interest are analytes that may be present in the biological sample;
obtaining estimates of the levels of the analytes of interest in the biological sample by applying the method according to claim 6; and
identifying the treatment for the subject based on the estimated levels of the analytes of interest in the biological sample.

9. A method of evaluating effectiveness of a treatment for a condition of a subject based on estimates of levels of analytes of interest in biological samples, the method comprising:
obtaining a first biological sample from the subject at a first time;
selecting the analytes of interest, wherein the analytes of interest are analytes that may be present in the biological sample and may be associated with the condition;
obtaining estimates of the levels of the analytes of interest in the first biological sample by applying the method according to claim 6;
applying a treatment to the subject;
obtaining a second biological sample from the subject at a second time;
obtaining estimates of the levels of the analytes of interest in the second biological sample by applying the method according to claim 6;
comparing the levels of the analytes of interest in the first and second biological samples;
evaluating the effectiveness of the treatment based on the comparison of the levels of the analytes of interest.

10. A method of training a model to estimate characteristics of a condition, the method comprising:
obtaining a first biological sample, wherein the first biological sample is suspected of exhibiting the condition;
obtaining a second biological sample, wherein the second biological sample is suspected of not exhibiting the condition;
obtaining liquid chromatographic and mass spectrometry data from the first and second biological samples;
selecting training analytes, wherein the training analytes are analytes that may be present in the first or second biological samples;
selecting precursors of the training analytes;
obtaining expected mass-to-charge ratios, predicted retention times and expected relative intensities of isotopes and product ions for each precursor of the training analytes;
preprocessing the liquid chromatographic and mass spectrometry data for each of the first and second biological samples into one or more arrays, wherein the arrays may be indexed based on mass-to-charge ratio and retention time;
generating a tensor data structure for each precursor of the training analytes for each of the first and second biological samples, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursors; and
training a model using the tensors corresponding to the precursors of the training analytes of the first and second biological samples to estimate characteristics of the condition.

11. A method of estimating characteristics of a condition of a subject, the method comprising:
obtaining a biological sample from the subject;
obtaining liquid chromatographic and mass spectrometry data from the biological sample;
selecting the analytes of interest, wherein the analytes of interest are analytes that may be present in the biological sample;
selecting precursors of the analytes of interest;
obtaining expected mass-to-charge ratios, predicted retention times and expected relative intensities of isotopes and product ions for each precursor of the training analytes;
preprocessing the liquid chromatographic and mass spectrometry data into one or more arrays, wherein the arrays may be indexed based on mass-to-charge ratio and retention time;
generating a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor; and
applying a model trained according to the method of claim 10 to estimate characteristics of the condition of the subject.

12. The method according to any of claims 1 to 11, wherein each tensor comprises a three-dimensional array of excerpts of preprocessed liquid chromatographic and mass spectrometry data comprising binned intensity data.

13. The method according to claim 12, wherein the binned intensity data comprises a two-dimensional space with axes corresponding to mass-to-charge ratio and retention time.

14. The method according to any of the previous claims, wherein selecting precursors of training analytes, analytes of interest or decoy analytes comprises identifying anticipated products of enzymatic cleavage of the training analytes, analytes of interest or decoy analytes.

15. The method according to claim 14, wherein selecting precursors of training analytes, analytes of interest or decoy analytes comprises identifying anticipated products of applying a Trypsin digest to the training analytes, analytes of interest or decoy analytes.

16. The method according to any of the previous claims, wherein the liquid chromatographic and mass spectrometry data from the biological sample comprises liquid chromatography-tandem mass spectrometry (LC-MS/MS) data.

17. The method according to any of the previous claims, wherein the liquid chromatographic and mass spectrometry data for the biological sample comprises SWATH mass spectrometry data.

18. The method according to any of the previous claims, wherein obtaining liquid chromatographic and mass spectrometry data for the biological sample comprises performing a liquid chromatography-tandem mass spectrometry (LC-MS/MS) technique on the biological sample.

19. The method according to any of claims 1 to 18, wherein obtaining liquid chromatographic and mass spectrometry data for the biological sample comprises applying a computational model to predict liquid chromatography retention times and/or expected relative intensities of isotopes or product ions.

20. The method according to any of claims 1 to 18, wherein obtaining liquid chromatographic and mass spectrometry data for the biological sample comprises applying a combination of performing a liquid chromatography-tandem mass spectrometry (LC-MS/MS) technique on the biological sample and applying a computational model to predict liquid chromatography retention times and/or expected relative intensities of isotopes or product ions.

21. The method according to any of the previous claims, wherein obtaining liquid chromatographic and mass spectrometry data for the biological sample comprises obtaining publicly available data.

22. The method according to any of claims 1 to 20, wherein obtaining liquid chromatographic and mass spectrometry data for the biological sample comprises applying a computational model to predict liquid chromatography retention times and/or relative intensities of isotopes or product ions.

23. The method according to any of claims 1 to 20, wherein obtaining liquid chromatographic and mass spectrometry data for the biological sample comprises applying a combination of at least obtaining publicly available data and applying a computational model to predict liquid chromatography retention times and/or relative intensities of isotopes or product ions.

24. The method according to any of the previous claims, wherein obtaining liquid chromatographic and mass spectrometry data for the biological sample comprises identifying liquid chromatographic retention times using an empirical approach or an iRT-based approach or a machine learning approach or a computational model-based approach or combinations thereof.

25. The method according to any of the previous claims, wherein the decoy analytes are not expected to be present in humans.

26. The method according to claim 25, wherein the decoy analytes are derived from non-human organisms.

27. The method according to any of the previous claims, further comprising generating a transition list for the precursors of the training analytes, the decoy analytes or the analytes of interest.

28. The method according to claim 27, wherein generating a tensor data structure for a precursor comprises using the transition list to generate a tensor.

29. The method according to any of the previous claims, wherein a tensor for a precursor corresponds to a transition list for the precursor, wherein a transition list comprises:
an ordered list of isotopes and product ions of the precursor;
an identification of whether the precursor corresponds to a training analyte, analyte of interest or decoy analyte;
a scan type for each isotope and product ion of the precursor;
a predicted liquid chromatographic retention time for each isotope and product ion of the precursor;
charge information for each isotope and product ion of the precursor;
mass information for each isotope and product ion of the precursor;
a mass to charge ratio for each isotope and product ion of the precursor; and
a ranking of expected mass spectrometry intensity data for each isotope and product ion of the precursor.

30. The method according to any of the previous claims, wherein a specified number of excerpts of the preprocessed liquid chromatographic and mass spectrometry data comprising intensity data are included in a tensor.

31. The method according to any of the previous claims, wherein the three-dimensional arrays of tensors comprise a plurality of two-dimensional arrays, wherein each two-dimensional array corresponds to an excerpt of the preprocessed liquid chromatographic and mass spectrometry data comprising intensity data centered around the expected mass-to-charge ratio and predicted retention time from an appropriate scan type of an isotope or product ion.

32. The method according to claim 31, wherein the preprocessed liquid chromatographic and mass spectrometry data comprising intensity data for an isotope or product ion is binned into elements of the corresponding two-dimensional array.

33. The method according to any of claims 31 to 32, wherein the plurality of two-dimensional arrays comprising a tensor comprises an ordered arrangement.

34. The method according to claim 33, wherein the plurality of two-dimensional arrays comprising each tensor corresponding to the precursors of the training analytes, analytes of interest and decoy analytes are ordered in the same manner.

35. The method according to claim 34, wherein the plurality of two-dimensional arrays comprising each tensor are ordered based on expected mass spectrographic intensities.

36. The method according to any of claims 31 to 35, wherein the plurality of two-dimensional arrays further comprises a two-dimensional array of weight information.

37. The method according to claim 36, wherein the weight information comprises a value at each two-dimensional position corresponding to a distance from a center position of the two-dimensional array of weight information.

38. The method according to claim 37, wherein the distance from center of the two-dimensional array comprises a distance from center based on mass-to-charge ratio.

39. The method according to claim 37, wherein the distance from center of the two-dimensional array comprises a distance from center in liquid chromatographic retention time.

40. The method according to claim 37, wherein the distance from center of the two-dimensional array comprises a combination of distance from center in mass-to-charge ratio and liquid chromatographic retention time.

41. The method according to any of the previous claims, wherein tensors corresponding to decoy analytes are associated with information indicating a level of zero in the biological sample.

42. The method according to any of the previous claims, wherein the model comprises a statistical model.

43. The method according to claim 42, wherein the model comprises a linear model.

44. The method according to any of claims 1 to 41, wherein the model comprises a computational model.

45. The method according to any of claims 1 to 42, wherein the model comprises a machine learning model.

46. The method according to claim 45, wherein the model comprises a tree-based model.

47. The method according to claim 45, wherein the model comprises a convolutional neural network.

48. The method according to claim 45, wherein the model comprises an artificial neural network.

49. The method according to claim 45, wherein the model comprises a deep learning network.

50. The method according to any of the previous claims, further comprising transforming the tensor data structures based on the model.

51. The method according to any of the previous claims, wherein training the model comprises applying an unsupervised learning technique to the model.

52. The method according to any of claims 1 to 50, wherein training the model comprises applying a semi-supervised learning technique to the model.

53. The method according to any of the previous claims, wherein training the model comprises applying a round robin training technique to the model.

54. The method according to any of the previous claims, wherein training the model comprises:
initially applying the model to obtain initial predictions regarding the training analytes; and
using at least a subset of the initial predictions to further train the model,
wherein initially applying the model comprises obtaining information about the confidence of the prediction generated by the model and the subset of initial predictions used to further train the model correspond to higher confidence predictions.

55. The method according to any of the previous claims, further comprising obtaining weak predictions of the presence of, or the levels of, the training analytes, the analytes of interest or the decoy analytes.

56. The method according to claim 55, further comprising using the weak predictions to train the model.

57. The method according to any of claims 55 to 56, wherein obtaining weak predictions comprises applying an algorithm to the liquid chromatographic and mass spectrometry data corresponding to precursors.

58. The method according to claim 57, wherein applying an algorithm to the liquid chromatographic and mass spectrometry data comprises applying an mProphet-based data processing technique to precursors.

59. The method according to any of claims 55 to 58, further comprising associating each weak prediction with a corresponding tensor.

60. The method according to any of the previous claims, further comprising obtaining scan types of the isotopes and product ions for precursors of training analytes, decoy analytes or analytes of interest.

61. The method according to claim 60, wherein preprocessing the liquid chromatographic mass spectrometry data into one or more arrays comprises preprocessing the data at least in part based on the scan types of the isotopes and product ions.

62. The method according to any of the previous claims, further comprising obtaining relative intensities of the isotopes and product ions for precursors of training analytes, decoy analytes or analytes of interest.

63. The method according to claim 62, wherein generating a tensor data structure for a precursor comprises extracting excerpts of the preprocessed liquid chromatographic and mass spectrometry data and ordering these excerpts based at least in part on the expected relative intensities of the isotopes and product ions of the precursor.

64. The method according to any of the previous claims, wherein the preprocessed liquid chromatographic and mass spectrometry data comprises transformed intensities.

65. The method according to any of the previous claims, wherein preprocessing the liquid chromatographic and mass spectroscopy data into one or more arrays comprises transforming mass spectrographic intensity data.

66. The method according to any of the previous claims, wherein training analytes, analytes of interest or decoy analytes comprise proteins.

67. The method according to any of the previous claims, wherein training analytes, analytes of interest or decoy analytes comprise peptides.

68. The method according to any of the previous claims, wherein precursors of analytes of interest comprise charged peptides.

69. The method according to any of claims 1 to 65, wherein training analytes, analytes of interest or decoy analytes comprise lipids.

70. The method according to any of claims 1 to 65, wherein training analytes, analytes of interest or decoy analytes comprise metabolites.

71. The method according to any of the previous claims, wherein a treatment comprises adjusting the subject's diet.

72. The method according to claim 71, wherein adjusting the subject's diet comprises instructing the subject to consume a specified food.

73. The method according to claim 71, wherein adjusting the subject's diet comprises instructing the subject to consume a specified food supplement.

74. The method according to claim 71, wherein adjusting the subject's diet comprises instructing the subject not to consume a specified food.

75. The method according to claim 71, wherein adjusting the subject's diet comprises instructing the subject not to consume a specified food supplement.

76. The method according to claim 71, wherein adjusting the subject's diet comprises instructing the subject to adhere to a specified feeding schedule.

77. The method according to any of the previous claims, wherein a treatment comprises recommending medication to the subject.

78. The method according to any of the previous claims, wherein a treatment comprises adjusting the subject's medication.

79. The method according to any of the previous claims, wherein a treatment comprises recommending behavior changes to the subject.

80. The method according to any of the previous claims, wherein a treatment comprises recommending referral to a specialist.

81. A method of providing a recurring evaluation and treatment for a subject suspected of having a condition, the method comprising:
(a) selecting analytes of interest, wherein the analytes of interest may be associated with the condition;
(b) obtaining a biological sample from the subject;
(c) obtaining estimates of the levels of the analytes of interest in the biological sample by applying the method according to claim 6;
(d) identifying the treatment for the subject based on the estimated levels of the analytes of interest in the biological sample;
(e) recommending the treatment to the subject for a specified period of time;
(f) providing recurring evaluation and treatment for the subject by repeating steps (a) through (e) one or more times.

82. The method according to claim 81, wherein the recurring intervention comprises a subscription service.

83. The method according to any of claims 81 to 82, wherein identifying the treatment for the subject comprises identifying changes to the subject's diet.

84. The method according to claim 81, wherein recommending the treatment to the subject comprises providing food-based treatment to the subject.

85. The method according to claim 84, wherein providing food-based treatment to the subject comprises a food subscription service.

86. The method according to claim 81, wherein the recurring intervention for the subject is repeated on a periodic basis determined at least in part based on the estimated levels of the analytes of interest in the biological sample.

87. The method according to any of claims 81 to 86, wherein the suspected condition is irritable bowel disease or non-alcoholic steatohepatitis or Crohn's disease or Rheumatoid arthritis or cardiovascular disease.

88. The method according to claim 1, wherein the model is trained using liquid chromatographic and mass spectrometry data obtained from a plurality of biological samples.

89. The method according to any of claims 1 or 88, wherein the biological sample comprises a plurality of distinct biological samples obtained from one or more subjects at one or more times.

90. The method according to any of claims 1 or 88 to 89, further comprising:
obtaining second liquid chromatographic and mass spectrometry data from a second biological sample;
preprocessing the second liquid chromatographic and mass spectrometry data from the second biological sample into one or more arrays, wherein the arrays may be indexed based on mass-to-charge ratio and retention time;
generating a tensor data structure for each precursor of the training analytes, wherein each tensor comprises a three-dimensional array of excerpts of the second preprocessed liquid chromatographic and mass spectrometry data comprising intensity data centered around the expected mass-to-charge ratios and the predicted retention times of the isotopes and product ions of the precursor; and
further training the model using the tensors corresponding to the precursors of the training analytes associated with the second biological sample to estimate a presence of precursors corresponding to analytes.

91. The method according to any of claims 3 to 4, wherein the model is trained using liquid chromatographic and mass spectrometry data obtained from a plurality of biological samples.

92. The method according to any of claims 3 to 4 or 91, wherein the biological sample comprises a plurality of distinct biological samples obtained from one or more subjects at one or more times.

93. The method according to any of claims 3 to 4 or 91 to 92, further comprising:
obtaining second liquid chromatographic and mass spectrometry data from a second biological sample;
preprocessing the second liquid chromatographic and mass spectrometry data from the second biological sample into one or more arrays, wherein the arrays may be indexed based on mass-to-charge ratio and retention time;
generating a tensor data structure for each precursor of the training analytes, wherein each tensor comprises a three-dimensional array of excerpts of the second preprocessed liquid chromatographic and mass spectrometry data comprising intensity data centered around the expected mass-to-charge ratios and the predicted retention times of the isotopes and product ions of the precursor; and
further training the model using the tensors corresponding to the precursors of the training analytes associated with the second biological sample to estimate levels of precursors corresponding to analytes.

94. The method according to claim 10, wherein the model is trained using liquid chromatographic and mass spectrometry data obtained from a plurality of biological samples suspected of exhibiting the condition.

95. The method according to any of claim 10 or 94, wherein the model is trained using liquid chromatographic and mass spectrometry data obtained from a plurality of biological samples suspected of not exhibiting the condition.

96. The method according to any of claims 10 or 94 to 95, wherein the first biological sample comprises a plurality of distinct biological samples suspected of exhibiting the condition and obtained from one or more subjects at one or more times.

97. The method according to any of claims 10 or 94 to 96, wherein the second biological sample comprises a plurality of distinct biological samples suspected of not exhibiting the condition and obtained from one or more subjects at one or more times.

98. The method according to any of claims 10 or 94 to 97, further comprising:
obtaining third liquid chromatographic and mass spectrometry data from a third biological sample suspected of exhibiting the condition;
obtaining fourth liquid chromatographic and mass spectrometry data from a fourth biological sample suspected of not exhibiting the condition;
preprocessing the second liquid chromatographic and mass spectrometry data from the third and fourth biological sample into one or more arrays, wherein the arrays may be indexed based on mass-to-charge ratio and retention time;
generating a tensor data structure for each precursor of the training analytes, wherein each tensor comprises a three-dimensional array of excerpts of the third and fourth preprocessed liquid chromatographic and mass spectrometry data comprising intensity data centered around the expected mass-to-charge ratios and the predicted retention times of the isotopes and product ions of the precursor; and
further training the model using the tensors corresponding to the precursors of the training analytes associated with the third and fourth biological samples to estimate characteristics of the condition.

99. A system comprising:
a processor comprising memory operably coupled to the processor, wherein the memory comprises instructions stored thereon, which, when executed by the processor, cause the processor to:
obtain training liquid chromatographic and mass spectrometry data corresponding to a training biological sample;
select training analytes, wherein the training analytes are analytes that may be present in the training biological sample;
select decoy analytes, wherein the decoy analytes are analytes that are not expected to be present in the training biological sample;
selecting precursors of the training analytes as well as precursors of the decoy analytes;
obtain expected mass-to-charge ratios, predicted retention times and expected relative intensities of isotopes and product ions for each precursor of the training analytes as well as for each precursor of the decoy analytes;
preprocess the training liquid chromatographic and mass spectrometry data into one or more arrays, wherein the arrays may be indexed based on mass-to-charge ratio and retention time;
generating a tensor data structure for each precursor of the training analytes and for each precursor of the decoy analytes, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor; and
train a model using the tensors corresponding to the precursors of the training analytes and the tensors corresponding to the decoy analytes to estimate a presence of precursors corresponding to analytes.

100. The system according to claim 99, wherein the memory further comprises instructions stored thereon, which, when executed by the processor, cause the processor to:
obtain liquid chromatographic and mass spectrometry data from a biological sample;
select analytes of interest, wherein the analytes of interest are analytes that may be present in the biological sample;
select precursors of the analytes of interest;
obtain expected mass-to-charge ratios and predicted retention times and expected relative intensities of isotopes and product ions for each precursor of the analytes of interest;
preprocess the liquid chromatographic and mass spectrometry data into one or more arrays, wherein the arrays may be indexed based on mass-to-charge ratio and retention time;
generate a tensor data structure for each precursor of the analytes of interest, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor;
apply the model to estimate the presence in the biological sample of the precursors corresponding to the analytes of interest; and
infer the presence of analytes of interest based on estimates of the presence of the precursors corresponding to the analytes of interest.

101. A system comprising:
a processor comprising memory operably coupled to the processor, wherein the memory comprises instructions stored thereon, which, when executed by the processor, cause the processor to:
obtain training liquid chromatographic and mass spectrometry data from a training biological sample;
select training analytes, wherein the training analytes are analytes that may be present in the training biological sample;
select precursors of the training analytes;
obtain expected mass-to-charge ratios, predicted retention times and expected relative intensities of isotopes and product ions for each precursor of the training analytes;
preprocess the training liquid chromatographic and mass spectrometry data into one or more arrays, wherein the arrays may be indexed based on mass-to-charge ratio and retention time;
generate a tensor data structure for each precursor of the training analytes, wherein each tensor comprises a three-dimensional array of excerpts of the preprocessed training liquid chromatographic and mass spectrometry data comprising intensity data centered at the expected mass-to-charge ratios and the predicted retention times of the isotopes and product ions of the precursor; and
train a model using the tensors corresponding to the precursors of the training analytes to estimate levels of precursors corresponding to analytes.

102. A system comprising:
a processor comprising memory operably coupled to the processor, wherein the memory comprises instructions stored thereon, which, when executed by the processor, cause the processor to:
obtain training liquid chromatographic and mass spectrometry data from a training biological sample;
select training analytes, wherein the training analytes are analytes that may be present in the training biological sample;
select decoy analytes, wherein the decoy analytes are analytes that are not expected to be present in the training biological sample;
select precursors of the training analytes as well as precursors of the decoy analytes;
obtain expected mass-to-charge ratios, predicted retention times and expected relative intensities of isotopes and product ions for each precursor of the training analytes as well as for each precursor of the decoy analytes;
preprocess the liquid chromatographic and mass spectrometry data into one or more arrays, wherein the arrays may be indexed based on mass-to-charge ratio and retention time;
generate a tensor data structure for each precursor of the training analytes and for each precursor of the decoy analytes, wherein each tensor comprises a three-dimensional array of excerpts of the preprocessed training liquid chromatographic and mass spectrometry data comprising intensity data centered around the expected mass-to-charge ratios and the predicted retention times of the isotopes and product ions of the precursor;
train a model using the tensors corresponding to the precursors of the training analytes and the tensors corresponding to the decoy analytes to estimate levels of precursors corresponding to analytes.

103. The system according to any of claims 101 or 102, wherein the memory further comprises instructions stored thereon, which, when executed by the processor, cause the processor to:
obtain liquid chromatographic and mass spectrometry data from a biological sample;
select analytes of interest, wherein the analytes of interest are analytes that may be present in the biological sample;
select precursors of the analytes of interest;
obtain expected mass-to-charge ratios, predicted retention times and expected relative intensities of isotopes and product ions for each precursor of the analytes of interest;
preprocess the liquid chromatographic and mass spectrometry data into one or more arrays, wherein the arrays may be indexed based on mass-to-charge ratio and retention time;
generate a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor;
apply the model to estimate the levels in the biological sample of the precursors corresponding to the analytes of interest; and
infer the levels of analytes of interest based on estimates of the levels of the precursors corresponding to the analytes of interest.

104. The system according to any of claims 101 or 102, wherein the memory further comprises instructions stored thereon, which, when executed by the processor, cause the processor to:
obtain a biological sample from a subject;
select analytes of interest, wherein the analytes of interest are analytes that may be present in the biological sample and may be associated with the condition;
apply the model to obtain estimates of levels of the analytes of interest in the biological sample; and
characterize a condition of the subject based on the estimated levels of the analytes of interest.

105. The system according to any of claims 101 or 102, wherein the memory further comprises instructions stored thereon, which, when executed by the processor, cause the processor to:
obtain a biological sample from a subject;
select analytes of interest, wherein the analytes of interest are analytes that may be present in the biological sample;
apply the model to obtain estimates of levels of the analytes of interest in the biological sample; and
identify a treatment for the subject based on the estimated levels of the analytes of interest in the biological sample.

106. The system according to any of claims 101 or 102, wherein the memory further comprises instructions stored thereon, which, when executed by the processor, cause the processor to:
obtain a first biological sample from a subject at a first time;
select analytes of interest, wherein the analytes of interest are analytes that may be present in the first biological sample and may be associated with a condition;
apply the model to obtain estimates of the levels of the analytes of interest in the first biological sample;
apply a treatment to the subject;
obtain a second biological sample from the subject at a second time;
apply the model to obtain estimates of levels of the analytes of interest in the second biological sample;
compare the levels of the analytes of interest in the first and second biological samples;
evaluate the effectiveness of the treatment based on the comparison of the levels of the analytes of interest.

107. A system comprising:
a processor comprising memory operably coupled to the processor, wherein the memory comprises instructions stored thereon, which, when executed by the processor, cause the processor to:
obtain a first training biological sample, wherein the first training biological sample is suspected of exhibiting a condition;
obtain a second training biological sample, wherein the second training biological sample is suspected of not exhibiting the condition;
obtain first and second liquid chromatographic and mass spectrometry data from the first and second biological samples;
select training analytes, wherein the training analytes are analytes that may be present in the first or second biological samples;
select precursors of the training analytes;
obtain expected mass-to-charge ratios, predicted retention times and expected relative intensities of isotopes and product ions for each precursor of the training analytes;
preprocess the first and second liquid chromatographic and mass spectrometry data for each of the first and second training biological samples into one or more arrays, wherein the arrays may be indexed based on mass-to-charge ratio and retention time;
generate a tensor data structure for each precursor of the training analytes for each of the first and second training biological samples, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursors; and
train a model using the tensors corresponding to the precursors of the training analytes of the first and second training biological samples to estimate characteristics of the condition.

108. The system according to any of claims 101 or 102, wherein the memory further comprises instructions stored thereon, which, when executed by the processor, cause the processor to:
obtain a biological sample from a subject;
obtain liquid chromatographic and mass spectrometry data from the biological sample;
select the analytes of interest, wherein the analytes of interest are analytes that may be present in the biological sample;
select precursors of the analytes of interest;
obtain expected mass-to-charge ratios, predicted retention times and expected relative intensities of isotopes and product ions for each precursor of the training analytes;
preprocess the liquid chromatographic and mass spectrometry data into one or more arrays, wherein the arrays may be indexed based on mass-to-charge ratio and retention time;
generate a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor; and
apply the model to estimate characteristics of the condition of the subject.

109. The system according to any of claims 99 to 108, wherein each tensor comprises a three-dimensional array of excerpts of preprocessed liquid chromatographic and mass spectrometry data comprising binned intensity data.

110. The system according to claim 109, wherein the binned intensity data comprises a two-dimensional space with axes corresponding to mass-to-charge ratio and retention time.

111. The system according to any of claims 99 to 110, wherein selecting precursors of training analytes, analytes of interest or decoy analytes comprises identifying anticipated products of enzymatic cleavage of analytes of interest.

112. The system according to any of claims 99 to 111, wherein selecting precursors of training analytes, analytes of interest or decoy analytes comprises identifying anticipated products of applying a Trypsin digest to the training analytes, analytes of interest or decoy analytes.

113. The system according to any of claims 99 to 112, wherein liquid chromatographic and mass spectrometry data comprises liquid chromatography-tandem mass spectrometry (LC-MS/MS) data.

114. The system according to any of claims 99 to 113, wherein the liquid chromatographic and mass spectrometry data for the biological sample comprises SWATH mass spectrometry data.

115. The system according to any of claims 99 to 114, wherein obtaining liquid chromatographic and mass spectrometry data comprises performing a liquid chromatography-tandem mass spectrometry (LC-MS/MS) technique on a biological sample.

116. The system according to any of claims 99 to 115, wherein obtaining liquid chromatographic and mass spectrometry data for the biological sample comprises applying a computational model to predict liquid chromatography retention times and/or expected relative intensities of isotopes or product ions.

117. The system according to any of claims 99 to 116, wherein obtaining liquid chromatographic and mass spectrometry data for the biological sample comprises applying a combination of performing a liquid chromatography-tandem mass spectrometry (LC-MS/MS) technique on the biological sample and applying a computational model to predict liquid chromatography retention times and/or expected relative intensities of isotopes or product ions.

118. The system according to any of claims 99 to 117, wherein obtaining liquid chromatographic and mass spectrometry data for a biological sample comprises obtaining publicly available data.

119. The system according to any of claims 99 to 118, wherein obtaining liquid chromatographic and mass spectrometry data for the biological sample comprises applying a computational model to predict liquid chromatography retention times and/or relative intensities of isotopes or product ions.

120. The system according to any of claims 99 to 119, wherein obtaining liquid chromatographic and mass spectrometry data for the biological sample comprises applying a combination of at least obtaining publicly available data and applying a computational model to predict liquid chromatography retention times and/or relative intensities of isotopes or product ions.

121. The system according to any of claims 99 to 120, wherein obtaining liquid chromatographic and mass spectrometry data for the biological sample comprises identifying liquid chromatographic retention times using an empirical approach or an iRT-based approach or a machine learning approach or a computational model-based approach or combinations thereof.

122. The system according to any of claims 99 to 121, wherein the decoy analytes are not expected to be present in humans.

123. The system according to claim 122, wherein the decoy analytes are derived from non-human organisms.

124. The system according to any of claims 99 to 123, further comprising generating a transition list for the precursors of the training analytes, the decoy analytes or the analytes of interest.

125. The system according to claim 124, wherein generating a tensor data structure for a precursor comprises using the transition list to generate a tensor.

126. The system according to any of claims 99 to 125, wherein a tensor for a precursor corresponds to a transition list for the precursor, wherein a transition list comprises:
an ordered list of isotopes and product ions of the precursor;
an identification of whether the precursor corresponds to a training analyte, analyte of interest or decoy analyte;
a scan type for each isotope and product ion of the precursor;
a predicted liquid chromatographic retention time for each isotope and product ion of the precursor;
charge information for each isotope and product ion of the precursor;
mass information for each isotope and product ion of the precursor;
a mass to charge ratio for each isotope and product ion of the precursor; and
a ranking of expected mass spectrometry intensity data for each isotope and product ion of the precursor.

127. The method according to any of claims 99 to 126, wherein a specified number of excerpts of the preprocessed liquid chromatographic and mass spectrometry data comprising intensity data are included in a tensor.

128. The system according to any of claims 99 to 127, wherein the three-dimensional arrays of tensors comprise a plurality of two-dimensional arrays, wherein each two-dimensional array corresponds to an excerpt of the preprocessed liquid chromatographic and mass spectrometry data comprising intensity data centered around the expected mass-to-charge ratio and predicted retention time from an appropriate scan type of an isotope or product ion.

129. The system according to claim 128, wherein the liquid chromatographic and mass spectrometry data comprising intensity data for an isotope or product ion is binned into elements of the corresponding two-dimensional array.

130. The system according to any of claims 128 to 129, wherein the plurality of two-dimensional arrays comprising a tensor comprises an ordered arrangement.

131. The system according to claim 130, wherein the plurality of two-dimensional arrays comprising each tensor corresponding to the precursors of the analytes of interest and to the precursors of the decoy analytes are ordered in the same manner.

132. The system according to claim 131, wherein the plurality of two-dimensional arrays comprising each tensor are ordered based on expected mass spectrographic intensities.

133. The system according to any of claims 128 to 132, wherein the plurality of two-dimensional arrays further comprises a two-dimensional array of weight information.

134. The system according to claim 133, wherein the weight information comprises a value at each two-dimensional position corresponding to a distance from a center position of the two-dimensional array of weight information.

135. The system according to claim 134, wherein the distance from center of the two-dimensional array comprises a distance from center based on mass-to-charge ratio.

136. The system according to claim 134, wherein the distance from center of the two-dimensional array comprises a distance from center in liquid chromatographic retention time.

137. The system according to claim 134, wherein the distance from center of the two-dimensional array comprises a combination of distance from center in mass-to-charge ratio and liquid chromatographic retention time.

138. The system according to any of claims 99 to 137, wherein tensors corresponding to decoy analytes are associated with information indicating a level of zero in the biological sample.

139. The system according to any of claims 99 to 138, wherein the model comprises a statistical model.

140. The system according to claim 139, wherein the model comprises a linear model.

141. The system according to any of claims 99 to 138, wherein the model comprises a computational model.

142. The system according to any of claims 99 to 141, wherein the model comprises a machine learning model.

143. The system according to claim 142, wherein the model comprises a tree-based model.

144. The system according to claim 142, wherein the model comprises a convolutional neural network.

145. The system according to claim 142, wherein the model comprises an artificial neural network.

146. The system according to claim 142, wherein the model comprises a deep learning network.

147. The system according to any of claims 99 to 146, further comprising transforming the tensor data structures based on the model.

148. The system according to any of claims 99 to 147, wherein training a model using at least a subset of the tensors comprises applying an unsupervised learning technique to the model.

149. The system according to any of claims 99 to 148, wherein training the model comprises applying a semi-supervised learning technique to the model.

150. The system according to any of claims 99 to 149, wherein training a model using at least a subset of the tensors comprises applying a semi-supervised learning technique to the model.

151. The system according to any of claims 99 to 150, wherein training a model using at least a subset of the tensors comprises applying a round robin training technique to the model.

152. The system according to any of claims 99 to 151, wherein training the model comprises:
initially applying the model to obtain initial predictions; and
using at least a subset of the initial predictions to further train the model,
wherein initially applying the model comprises obtaining information about the confidence of the prediction generated by the model and the subset of initial predictions used to further train the model correspond to higher confidence predictions.

153. The system according to any of claims 99 to 152, further comprising obtaining weak predictions of the presence of, or the levels of, the training analytes, the analytes of interest or the decoy analytes.

154. The system according to claim 153, further comprising using the weak predictions to train the model.

155. The system according to any of claims 153 to 154, wherein obtaining weak predictions comprises applying an algorithm to the liquid chromatographic and mass spectrometry data corresponding to precursors.

156. The system according to claim 155, wherein applying an algorithm to the liquid chromatographic and mass spectrometry data comprises applying an mProphet-based data processing technique to precursors.

157. The system according to any of claims 153 to 156, further comprising associating each weak prediction with a corresponding tensor.

158. The system according to any of claims 99 to 157, further comprising obtaining scan types of the isotopes and product ions for precursors of training analytes, decoy analytes or analytes of interest.

159. The system according to claim 158, wherein preprocessing the liquid chromatographic mass spectrometry data into one or more arrays comprises preprocessing the data at least in part based on the scan types of the isotopes and product ions.

160. The system according to any of claims 99 to 159, further comprising obtaining relative intensities of the isotopes and product ions for precursors of training analytes, decoy analytes or analytes of interest.

161. The system according to claim 160, wherein generating a tensor data structure for a precursor comprises extracting excerpts of the preprocessed liquid chromatographic and mass spectrometry data based at least in part on the relative intensities of the isotopes and product ions of the precursor.

162. The system according to any of claims 99 to 161, wherein the preprocessed liquid chromatographic and mass spectrometry data comprises transformed intensities.

163. The system according to any claims 99 to 162, wherein preprocessing the liquid chromatographic and mass spectroscopy data into one or more arrays comprises transforming mass spectrographic intensity data.

164. The system according to any of claims 99 to 163, wherein training analytes, analytes of interest or decoy analytes comprise proteins.

165. The system according to any of claims 99 to 164, wherein training analytes, analytes of interest or decoy analytes comprise peptides.

166. The system according to any of claims 99 to 165, wherein precursors of analytes of interest comprise charged peptides.

167. The system according to any of claims 99 to 164, wherein training analytes, analytes of interest or decoy analytes comprise lipids.

168. The system according to any of claims 99 to 164, wherein training analytes, analytes of interest or decoy analytes comprise metabolites.

169. The system according to any of claims 99 to 168, wherein a treatment comprises adjusting the subject's diet.

170. The system according to claim 169, wherein adjusting the subject's diet comprises instructing the subject to consume a specified food.

171. The system according to claim 169, wherein adjusting the subject's diet comprises instructing the subject to consume a specified food supplement.

172. The system according to claim 169, wherein adjusting the subject's diet comprises instructing the subject not to consume a specified food.

173. The system according to claim 169, wherein adjusting the subject's diet comprises instructing the subject not to consume a specified food supplement.

174. The system according to claim 169, wherein adjusting the subject's diet comprises instructing the subject to adhere to a specified feeding schedule.

175. The system according to any of claims 99 to 174, wherein a treatment comprises recommending medication to the subject.

176. The system according to any of claims 99 to 175, wherein a treatment comprises adjusting the subject's medication.

177. The claims 99 to 174 according to any of claims 99 to 176, wherein a treatment comprises recommending behavior changes to the subject.

178. The claims 99 to 174 according to any of the previous claims, wherein a treatment comprises recommending referral to a specialist.

179. The system according to any of claims 101 or 102, wherein the memory further comprises instructions stored thereon, which, when executed by the processor, cause the processor to:
(a) select analytes of interest, wherein the analytes of interest may be associated with the condition;
(b) obtain a biological sample from the subject;
(c) apply the model to obtain estimates of the levels of the analytes of interest in the biological sample;
(d) identify the treatment for the subject based on the estimated levels of the analytes of interest in the biological sample;
(e) recommend the treatment to the subject for a specified period of time;
(f) provide recurring evaluation and treatment for the subject by repeating steps (a) through (e) one or more times.

180. The system according to claim 179, wherein the recurring intervention comprises a subscription service.

181. The system according to any of claims 179 to 180, wherein identifying the treatment for the subject comprises identifying changes to the subject's diet.

182. The system according to claim 181, wherein recommending the treatment to the subject comprises providing food-based treatment to the subject.

183. The system according to claim 182, wherein providing food-based treatment to the subject comprises a food subscription service.

184. The system according to claim 181, wherein the recurring intervention for the subject is repeated on a periodic basis determined at least in part based on the estimated levels of the analytes of interest in the biological sample.

185. The system according to any of claims 181 to 184, wherein the suspected condition is irritable bowel disease or non-alcoholic steatohepatitis or Crohn's disease or Rheumatoid arthritis or cardiovascular disease.

186. The system according to claim 99, wherein the model is trained using liquid chromatographic and mass spectrometry data obtained from a plurality of biological samples.

187. The system according to any of claims 99 or 186, wherein the biological sample comprises a plurality of distinct biological samples obtained from one or more subjects at one or more times.

188. The system according to any of claims 99 or 186 to 187, wherein the memory further comprises instructions stored thereon, which, when executed by the processor, cause the processor to:
obtain second liquid chromatographic and mass spectrometry data from a second biological sample;
preprocess the second liquid chromatographic and mass spectrometry data from the second biological sample into one or more arrays, wherein the arrays may be indexed based on mass-to-charge ratio and retention time;
generate a tensor data structure for each precursor of the training analytes, wherein each tensor comprises a three-dimensional array of excerpts of the second preprocessed liquid chromatographic and mass spectrometry data comprising intensity data centered around the expected mass-to-charge ratios and the predicted retention times of the isotopes and product ions of the precursor; and
further train the model using the tensors corresponding to the precursors of the training analytes associated with the second biological sample to estimate a presence of precursors corresponding to analytes.

189. The system according to any of claims 101 to 102, wherein the model is trained using liquid chromatographic and mass spectrometry data obtained from a plurality of biological samples.

190. The system according to any of claims 101 to 102 or 189, wherein the biological sample comprises a plurality of distinct biological samples obtained from one or more subjects at one or more times.

191. The system according to any of claims 101 to 102 or 189 to 190, wherein the memory further comprises instructions stored thereon, which, when executed by the processor, cause the processor to:
obtaining second liquid chromatographic and mass spectrometry data from a second biological sample;
preprocessing the second liquid chromatographic and mass spectrometry data from the second biological sample into one or more arrays, wherein the arrays may be indexed based on mass-to-charge ratio and retention time;
generating a tensor data structure for each precursor of the training analytes, wherein each tensor comprises a three-dimensional array of excerpts of the second preprocessed liquid chromatographic and mass spectrometry data comprising intensity data centered around the expected mass-to-charge ratios and the predicted retention times of the isotopes and product ions of the precursor; and
further training the model using the tensors corresponding to the precursors of the training analytes associated with the second biological sample to estimate levels of precursors corresponding to analytes.

192. The system according to claim 107, wherein the model is trained using liquid chromatographic and mass spectrometry data obtained from a plurality of biological samples suspected of exhibiting the condition.

193. The system according to any of claims 107 or 192, wherein the model is trained using liquid chromatographic and mass spectrometry data obtained from a plurality of biological samples suspected of not exhibiting the condition.

194. The system according to any of claims 107 or 192 to 193, wherein the first biological sample comprises a plurality of distinct biological samples suspected of exhibiting the condition and obtained from one or more subjects at one or more times.

195. The system according to any of claims 107 or 192 to 194, wherein the second biological sample comprises a plurality of distinct biological samples suspected of not exhibiting the condition and obtained from one or more subjects at one or more times.

196. The system according to any of claims 107 or 192 to 194, wherein the memory further comprises instructions stored thereon, which, when executed by the processor, cause the processor to:
obtaining third liquid chromatographic and mass spectrometry data from a third biological sample suspected of exhibiting the condition;
obtaining fourth liquid chromatographic and mass spectrometry data from a fourth biological sample suspected of not exhibiting the condition;
preprocessing the second liquid chromatographic and mass spectrometry data from the third and fourth biological sample into one or more arrays, wherein the arrays may be indexed based on mass-to-charge ratio and retention time;
generating a tensor data structure for each precursor of the training analytes, wherein each tensor comprises a three-dimensional array of excerpts of the third and fourth preprocessed liquid chromatographic and mass spectrometry data comprising intensity data centered around the expected mass-to-charge ratios and the predicted retention times of the isotopes and product ions of the precursor; and
further training the model using the tensors corresponding to the precursors of the training analytes associated with the third and fourth biological samples to estimate characteristics of the condition.

197. One or more non-transitory computer-readable storage media storing instructions which, when executed by at least one processor, cause the at least one processor to perform operations comprising:
obtaining liquid chromatographic and mass spectrometry data from the biological sample;
selecting training analytes, wherein the training analytes are analytes that may be present in the biological sample;
selecting decoy analytes, wherein the decoy analytes are analytes that are not expected to be present in the biological sample;
selecting precursors of the training analytes as well as precursors of the decoy analytes;
obtaining expected mass-to-charge ratios, predicted retention times and expected relative intensities of isotopes and product ions for each precursor of the training analytes as well as for each precursor of the decoy analytes;
preprocessing the liquid chromatographic and mass spectrometry data into one or more arrays, wherein the arrays may be indexed based on mass-to-charge ratio and retention time;
generating a tensor data structure for each precursor of the training analytes and for each precursor of the decoy analytes, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor; and
training a model using the tensors corresponding to the precursors of the training analytes and the tensors corresponding to the decoy analytes to estimate a presence of precursors corresponding to analytes.

198. The one or more non-transitory computer-readable storage medium of claim 197, wherein the instructions, when executed by the at least one processor, cause the at least one processor to perform further operations comprising:
obtaining liquid chromatographic and mass spectrometry data from a biological sample;
selecting analytes of interest, wherein the analytes of interest are analytes that may be present in the biological sample;
selecting precursors of the analytes of interest;
obtaining expected mass-to-charge ratios and predicted retention times and expected relative intensities of isotopes and product ions for each precursor of the analytes of interest;
preprocessing the liquid chromatographic and mass spectrometry data into one or more arrays, wherein the arrays may be indexed based on mass-to-charge ratio and retention time;
generating a tensor data structure for each precursor of the analytes of interest, wherein each tensor comprises a three-dimensional array of excerpts of the preprocessed liquid chromatographic and mass spectrometry data comprising intensity data centered around the expected mass-to-charge ratios and the predicted retention times of the isotopes and product ions of the precursor;
applying the model to estimate the presence in the biological sample of the precursors corresponding to the analytes of interest; and
inferring the presence of analytes of interest based on estimates of the presence of the precursors corresponding to the analytes of interest.

199. One or more non-transitory computer-readable storage media storing instructions which, when executed by at least one processor, cause the at least one processor to perform operations comprising:
obtaining training liquid chromatographic and mass spectrometry data from a training biological sample;
selecting training analytes, wherein the training analytes are analytes that may be present in the training biological sample;
selecting precursors of the training analytes;
obtaining expected mass-to-charge ratios, predicted retention times and expected relative intensities of isotopes and product ions for each precursor of the training analytes;
preprocessing the training liquid chromatographic and mass spectrometry data into one or more arrays, wherein the arrays may be indexed based on mass-to-charge ratio and retention time;
generating a tensor data structure for each precursor of the training analytes, wherein each tensor comprises a three-dimensional array of excerpts of the preprocessed training liquid chromatographic and mass spectrometry data comprising intensity data centered at the expected mass-to-charge ratios and the predicted retention times of the isotopes and product ions of the precursor; and
training a model using the tensors corresponding to the precursors of the training analytes to estimate levels of precursors corresponding to analytes.

200. One or more non-transitory computer-readable storage media storing instructions which, when executed by at least one processor, cause the at least one processor to perform operations comprising:
obtaining training liquid chromatographic and mass spectrometry data from a training biological sample;
selecting training analytes, wherein the training analytes are analytes that may be present in the training biological sample;
selecting decoy analytes, wherein the decoy analytes are analytes that are not expected to be present in the training biological sample;
selecting precursors of the training analytes as well as precursors of the decoy analytes;
obtaining expected mass-to-charge ratios, predicted retention times and expected relative intensities of isotopes and product ions for each precursor of the training analytes as well as for each precursor of the decoy analytes;
preprocessing the liquid chromatographic and mass spectrometry data into one or more arrays, wherein the arrays may be indexed based on mass-to-charge ratio and retention time;
generating a tensor data structure for each precursor of the training analytes and for each precursor of the decoy analytes, wherein each tensor comprises a three-dimensional array of excerpts of the preprocessed training liquid chromatographic and mass spectrometry data comprising intensity data centered around the expected mass-to-charge ratios and the predicted retention times of the isotopes and product ions of the precursor; and
training a model using the tensors corresponding to the precursors of the training analytes and the tensors corresponding to the decoy analytes to estimate levels of precursors corresponding to analytes.

201. The one or more non-transitory computer-readable storage medium of claim 199 or 200, wherein the instructions, when executed by the at least one processor, cause the at least one processor to perform further operations comprising:
obtaining liquid chromatographic and mass spectrometry data from a biological sample;
selecting analytes of interest, wherein the analytes of interest are analytes that may be present in the biological sample;
selecting precursors of the analytes of interest;
obtaining expected mass-to-charge ratios, predicted retention times and expected relative intensities of isotopes and product ions for each precursor of the analytes of interest;
preprocessing the liquid chromatographic and mass spectrometry data into one or more arrays, wherein the arrays may be indexed based on mass-to-charge ratio and retention time;
generating a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor;
applying the model to estimate the levels in the biological sample of the precursors corresponding to the analytes of interest; and
inferring the levels of analytes of interest based on estimates of the levels of the precursors corresponding to the analytes of interest.

202. The one or more non-transitory computer-readable storage medium of claim 199 or 200, wherein the instructions, when executed by the at least one processor, cause the at least one processor to perform further operations comprising:
obtaining a biological sample from a subject;
selecting analytes of interest, wherein the analytes of interest are analytes that may be present in the biological sample and may be associated with the condition;
applying the model to obtain estimates of levels of the analytes of interest in the biological sample; and
characterizing a condition of the subject based on the estimated levels of the analytes of interest.

203. The one or more non-transitory computer-readable storage medium of claim 199 or 200, wherein the instructions, when executed by the at least one processor, cause the at least one processor to perform further operations comprising:
obtaining a biological sample from a subject;
selecting analytes of interest, wherein the analytes of interest are analytes that may be present in the biological sample;
applying the model to obtain estimates of levels of the analytes of interest in the biological sample; and
identifying a treatment for the subject based on the estimated levels of the analytes of interest in the biological sample.

204. The one or more non-transitory computer-readable storage medium of claim 199 or 200, wherein the instructions, when executed by the at least one processor, cause the at least one processor to perform further operations comprising:
obtaining a first biological sample from a subject at a first time;
selecting analytes of interest, wherein the analytes of interest are analytes that may be present in the first biological sample and may be associated with a condition;
applying the model to obtain estimates of the levels of the analytes of interest in the first biological sample;
applying a treatment to the subject;
obtaining a second biological sample from the subject at a second time;
applying the model to obtain estimates of levels of the analytes of interest in the second biological sample;
comparing the levels of the analytes of interest in the first and second biological samples;
evaluating the effectiveness of the treatment based on the comparison of the levels of the analytes of interest.

205. One or more non-transitory computer-readable storage media storing instructions which, when executed by at least one processor, cause the at least one processor to perform operations comprising:
obtaining a first training biological sample, wherein the first training biological sample is suspected of exhibiting a condition;
obtaining a second training biological sample, wherein the second training biological sample is suspected of not exhibiting the condition;
obtaining first and second liquid chromatographic and mass spectrometry data from the first and second biological samples;
selecting training analytes, wherein the training analytes are analytes that may be present in the first or second biological samples;
selecting precursors of the training analytes;
obtaining expected mass-to-charge ratios, predicted retention times and expected relative intensities of isotopes and product ions for each precursor of the training analytes;
preprocessing the first and second liquid chromatographic and mass spectrometry data for each of the first and second training biological samples into one or more arrays, wherein the arrays may be indexed based on mass-to-charge ratio and retention time;
generating a tensor data structure for each precursor of the training analytes for each of the first and second training biological samples, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursors;
training a model using the tensors corresponding to the precursors of the training analytes of the first and second training biological samples to estimate characteristics of the condition.

206. The one or more non-transitory computer-readable storage medium of claim 205, wherein the instructions, when executed by the at least one processor, cause the at least one processor to perform further operations comprising:
obtaining a biological sample from a subject;
obtaining liquid chromatographic and mass spectrometry data from the biological sample;
selecting the analytes of interest, wherein the analytes of interest are analytes that may be present in the biological sample;
selecting precursors of the analytes of interest;
obtaining expected mass-to-charge ratios, predicted retention times and expected relative intensities of isotopes and product ions for each precursor of the training analytes;
preprocessing the liquid chromatographic and mass spectrometry data into one or more arrays, wherein the arrays may be indexed based on mass-to-charge ratio and retention time;
generating a tensor data structure for each precursor of the analytes of interest, wherein each tensor comprises a three-dimensional array of excerpts of the preprocessed liquid chromatographic and mass spectrometry data comprising intensity data centered at the expected mass-to-charge ratios and the predicted retention times of the isotopes and product ions of the precursor; and
applying the model to estimate characteristics of the condition of the subject.

207. The one or more non-transitory computer-readable storage media according to any of claims 197 to 206, wherein each tensor comprises a three-dimensional array of excerpts of preprocessed liquid chromatographic and mass spectrometry data comprising binned intensity data.

208. The one or more non-transitory computer-readable storage media according to claim 207, wherein the binned intensity data comprises a two-dimensional space with axes corresponding to mass-to-charge ratio and retention time.

209. The one or more non-transitory computer-readable storage media according to any of claims 197 to 208, wherein selecting precursors of training analytes, analytes of interest or decoy analytes comprises identifying anticipated products of enzymatic cleavage of analytes of interest.

210. The one or more non-transitory computer-readable storage media according to claim 209, wherein selecting precursors of training analytes, analytes of interest or decoy analytes comprises identifying anticipated products of applying a Trypsin digest to the training analytes, analytes of interest or decoy analytes.

211. The one or more non-transitory computer-readable storage media according to any of claims 197 to 210, wherein the liquid chromatographic and mass spectrometry data from the biological sample comprises liquid chromatography-tandem mass spectrometry (LC-MS/MS) data.

212. The one or more non-transitory computer-readable storage media according to any of claims 197 to 211, wherein the liquid chromatographic and mass spectrometry data for the biological sample comprises SWATH mass spectrometry data.

213. The one or more non-transitory computer-readable storage media according to any of claims 197 to 212, wherein obtaining liquid chromatographic and mass spectrometry data for the biological sample comprises performing a liquid chromatography-tandem mass spectrometry (LC-MS/MS) technique on the biological sample.

214. The one or more non-transitory computer-readable storage media according to any of claims 197 to 213, wherein obtaining liquid chromatographic and mass spectrometry data for the biological sample comprises applying a computational model to predict liquid chromatography retention times and/or expected relative intensities of isotopes or product ions.

215. The one or more non-transitory computer-readable storage media according to any of claims 197 to 214, wherein obtaining liquid chromatographic and mass spectrometry data for the biological sample comprises applying a combination of performing a liquid chromatography-tandem mass spectrometry (LC-MS/MS) technique on the biological sample and applying a computational model to predict liquid chromatography retention times and/or expected relative intensities of isotopes or product ions.

216. The one or more non-transitory computer-readable storage media according to any of claims 197 to 215, wherein obtaining liquid chromatographic and mass spectrometry data for the biological sample comprises obtaining publicly available data.

217. The one or more non-transitory computer-readable storage media according to any of claims 197 to 216, wherein obtaining liquid chromatographic and mass spectrometry data for the biological sample comprises applying a computational model to predict liquid chromatography retention times and/or relative intensities of isotopes or product ions.

218. The one or more non-transitory computer-readable storage media according to any of claims 197 to 217, wherein obtaining liquid chromatographic and mass spectrometry data for the biological sample comprises applying a combination of at least obtaining publicly available data and applying a computational model to predict liquid chromatography retention times and/or relative intensities of isotopes or product ions.

219. The one or more non-transitory computer-readable storage media according to any of claims 197 to 218, wherein obtaining liquid chromatographic and mass spectrometry data for the biological sample comprises identifying liquid chromatographic retention times using an empirical approach or an iRT-based approach or a machine learning approach or a computational model-based approach or combinations thereof.

220. The one or more non-transitory computer-readable storage media according to any of claims 197 to 219, wherein the decoy analytes are not expected to be present in humans.

221. The one or more non-transitory computer-readable storage media according to claim 220, wherein the decoy analytes are derived from non-human organisms.

222. The one or more non-transitory computer-readable storage media according to any of claims 197 to 221, further comprising generating a transition list for the precursors of the training analytes, the decoy analytes or the analytes of interest.

223. The one or more non-transitory computer-readable storage media according to claim 222, wherein generating a tensor data structure for a precursor comprises using the transition list to generate a tensor.

224. The one or more non-transitory computer-readable storage media according to any of claims 197 to 223, wherein a tensor for a precursor corresponds to a transition list for the precursor, wherein a transition list comprises:
an ordered list of isotopes and product ions of the precursor;
an identification of whether the precursor corresponds to a training analyte, analyte of interest or decoy analyte;
a scan type for each isotope and product ion of the precursor;
a predicted liquid chromatographic retention time for each isotope and product ion of the precursor;
charge information for each isotope and product ion of the precursor;
mass information for each isotope and product ion of the precursor;
a mass to charge ratio for each isotope and product ion of the precursor; and
a ranking of expected mass spectrometry intensity data for each isotope and product ion of the precursor.

225. The one or more non-transitory computer-readable storage media according to any of claims 197 to 224, wherein a specified number of excerpts of the preprocessed liquid chromatographic and mass spectrometry data comprising intensity data are included in a tensor.

226. The one or more non-transitory computer-readable storage media according to any of claims 197 to 225, wherein the three-dimensional arrays of tensors comprise a plurality of two-dimensional arrays, wherein each two-dimensional array corresponds to an excerpt of the preprocessed liquid chromatographic and mass spectrometry data comprising intensity data centered around the expected mass-to-charge ratio and predicted retention time from an appropriate scan type of an isotope or product ion.

227. The one or more non-transitory computer-readable storage media according to claim 226, wherein the preprocessed liquid chromatographic and mass spectrometry data comprising intensity data for an isotope or product ion is binned into elements of the corresponding two-dimensional array.

228. The one or more non-transitory computer-readable storage media according to any of claims 226 to 227, wherein the plurality of two-dimensional arrays comprising a tensor comprises an ordered arrangement.

229. The one or more non-transitory computer-readable storage media according to claim 228, wherein the plurality of two-dimensional arrays comprising each tensor corresponding to the precursors of the training analytes, analytes of interest and decoy analytes are ordered in the same manner.

230. The one or more non-transitory computer-readable storage media according to claim 229, wherein the plurality of two-dimensional arrays comprising each tensor are ordered based on expected mass spectrographic intensities.

231. The one or more non-transitory computer-readable storage media according to any of claims 226 to 230, wherein the plurality of two-dimensional arrays further comprises a two-dimensional array of weight information.

232. The one or more non-transitory computer-readable storage media according to claim 231, wherein the weight information comprises a value at each two-dimensional position corresponding to a distance from a center position of the two-dimensional array of weight information.

233. The one or more non-transitory computer-readable storage media according to claim 232, wherein the distance from center of the two-dimensional array comprises a distance from center based on mass-to-charge ratio.

234. The one or more non-transitory computer-readable storage media according to claim 232, wherein the distance from center of the two-dimensional array comprises a distance from center in liquid chromatographic retention time.

235. The one or more non-transitory computer-readable storage media according to claim 232, wherein the distance from center of the two-dimensional array comprises a combination of distance from center in mass-to-charge ratio and liquid chromatographic retention time.

236. The one or more non-transitory computer-readable storage media according to any claims 197 to 235, wherein tensors corresponding to decoy analytes are associated with information indicating a level of zero in the biological sample.

237. The one or more non-transitory computer-readable storage media according to any of claims 197 to 236, wherein the model comprises a statistical model.

238. The one or more non-transitory computer-readable storage media according to claim 237, wherein the model comprises a linear model.

239. The one or more non-transitory computer-readable storage media according to any of claims 197 to 236, wherein the model comprises a computational model.

240. The one or more non-transitory computer-readable storage media according to any of claims 197 to 237, wherein the model comprises a machine learning model.

241. The one or more non-transitory computer-readable storage media according to claim 240, wherein the model comprises a tree-based model.

242. The one or more non-transitory computer-readable storage media according to claim 240, wherein the model comprises a convolutional neural network.

243. The one or more non-transitory computer-readable storage media according to claim 240, wherein the model comprises an artificial neural network.

244. The one or more non-transitory computer-readable storage media according to claim 240, wherein the model comprises a deep learning network.

245. The one or more non-transitory computer-readable storage media according to any of claims 197 to 244, further comprising transforming the tensor data structures based on the model.

246. The one or more non-transitory computer-readable storage media according to any of claims 197 to 245, wherein training the model comprises applying an unsupervised learning technique to the model.

247. The one or more non-transitory computer-readable storage media according to any of claims 197 to 245, wherein training the model comprises applying a semi-supervised learning technique to the model.

248. The one or more non-transitory computer-readable storage media according to any of claims 197 to 247, wherein training the model comprises applying a round robin training technique to the model.

249. The one or more non-transitory computer-readable storage media according to any of claims 197 to 248, wherein training the model comprises:
initially applying the model to obtain initial predictions regarding the training analytes; and
using at least a subset of the initial predictions to further train the model,
wherein initially applying the model comprises obtaining information about the confidence of the prediction generated by the model and the subset of initial predictions used to further train the model correspond to higher confidence predictions.

250. The one or more non-transitory computer-readable storage media according to any of claims 197 to 249, further comprising obtaining weak predictions of the presence of, or the levels of, the training analytes, the analytes of interest or the decoy analytes.

251. The one or more non-transitory computer-readable storage media according to claim 250, further comprising using the weak predictions to train the model.

252. The one or more non-transitory computer-readable storage media according to any of claims 250 to 251, wherein obtaining weak predictions comprises applying an algorithm to the liquid chromatographic and mass spectrometry data corresponding to precursors.

253. The one or more non-transitory computer-readable storage media according to claim 252, wherein applying an algorithm to the liquid chromatographic and mass spectrometry data comprises applying an mProphet-based data processing technique to precursors.

254. The one or more non-transitory computer-readable storage media according to any of claims 250 to 253, further comprising associating each weak prediction with a corresponding tensor.

255. The one or more non-transitory computer-readable storage media according to any of claims 197 to 254, further comprising obtaining scan types of the isotopes and product ions for precursors of training analytes, decoy analytes or analytes of interest.

256. The one or more non-transitory computer-readable storage media according to claim 255, wherein preprocessing the liquid chromatographic mass spectrometry data into one or more arrays comprises preprocessing the data at least in part based on the scan types of the isotopes and product ions.

257. The one or more non-transitory computer-readable storage media according to any of claims 197 to 256, further comprising obtaining relative intensities of the isotopes and product ions for precursors of training analytes, decoy analytes or analytes of interest.

258. The one or more non-transitory computer-readable storage media according to claim 257, wherein generating a tensor data structure for a precursor comprises extracting excerpts of the preprocessed liquid chromatographic and mass spectrometry data based at least in part on the relative intensities of the isotopes and product ions of the precursor.

259. The one or more non-transitory computer-readable storage media according to any of claims 197 to 258, wherein the preprocessed liquid chromatographic and mass spectrometry data comprises transformed intensities.

260. The one or more non-transitory computer-readable storage media according to any of claims 197 to 259, wherein preprocessing the liquid chromatographic and mass spectroscopy data into one or more arrays comprises transforming mass spectrographic intensity data.

261. The one or more non-transitory computer-readable storage media according to any of claims 197 to 260, wherein training analytes, analytes of interest or decoy analytes comprise proteins.

262. The one or more non-transitory computer-readable storage media according to any of claims 197 to 261, wherein training analytes, analytes of interest or decoy analytes comprise peptides.

263. The one or more non-transitory computer-readable storage media according to any of claims 197 to 262, wherein precursors of analytes of interest comprise charged peptides.

264. The one or more non-transitory computer-readable storage media according to any of claims 197 to 260, wherein training analytes, analytes of interest or decoy analytes comprise lipids.

265. The one or more non-transitory computer-readable storage media according to any of claims 197 to 260, wherein training analytes, analytes of interest or decoy analytes comprise metabolites.

266. The one or more non-transitory computer-readable storage media according to any of claims 197 to 265, wherein a treatment comprises adjusting the subject's diet.

267. The one or more non-transitory computer-readable storage media according to claim 266, wherein adjusting the subject's diet comprises instructing the subject to consume a specified food.

268. The one or more non-transitory computer-readable storage media according to claim 266, wherein adjusting the subject's diet comprises instructing the subject to consume a specified food supplement.

269. The one or more non-transitory computer-readable storage media according to claim 266, wherein adjusting the subject's diet comprises instructing the subject not to consume a specified food.

270. The one or more non-transitory computer-readable storage media according to claim 266, wherein adjusting the subject's diet comprises instructing the subject not to consume a specified food supplement.

271. The one or more non-transitory computer-readable storage media according to claim 266, wherein adjusting the subject's diet comprises instructing the subject to adhere to a specified feeding schedule.

272. The one or more non-transitory computer-readable storage media according to any of claims 197 to 271, wherein a treatment comprises recommending medication to the subject.

273. The one or more non-transitory computer-readable storage media according to any of claims 197 to 272, wherein a treatment comprises adjusting the subject's medication.

274. The one or more non-transitory computer-readable storage media according to any of claims 197 to 273, wherein a treatment comprises recommending behavior changes to the subject.

275. The one or more non-transitory computer-readable storage media according to any of claims 197 to 274, wherein a treatment comprises recommending referral to a specialist.

276. The one or more non-transitory computer-readable storage media of claim 199 or 200, wherein the instructions, when executed by the at least one processor, cause the at least one processor to perform further operations comprising:
(a) selecting analytes of interest, wherein the analytes of interest may be associated with the condition;
(b) obtaining a biological sample from the subject;
(c) applying the model to obtaining estimates of the levels of the analytes of interest in the biological sample;
(d) identifying the treatment for the subject based on the estimated levels of the analytes of interest in the biological sample;
(e) recommending the treatment to the subject for a specified period of time;
(f) providing recurring evaluation and treatment for the subject by repeating steps (a) through (e) one or more times.

277. The one or more non-transitory computer-readable storage media according to claim 276, wherein the recurring intervention comprises a subscription service.

278. The one or more non-transitory computer-readable storage media according to any of claims 276 to 277, wherein identifying the treatment for the subject comprises identifying changes to the subject's diet.

279. The one or more non-transitory computer-readable storage media according to claim 276, wherein recommending the treatment to the subject comprises providing food-based treatment to the subject.

280. The one or more non-transitory computer-readable storage media according to claim 279, wherein providing food-based treatment to the subject comprises a food subscription service.

281. The one or more non-transitory computer-readable storage media according to claim 276, wherein the recurring intervention for the subject is repeated on a periodic basis determined at least in part based on the estimated levels of the analytes of interest in the biological sample.

282. The one or more non-transitory computer-readable storage media according to any of claims 276 to 281, wherein the suspected condition is irritable bowel disease or non-alcoholic steatohepatitis or Crohn's disease or Rheumatoid arthritis or cardiovascular disease.

283. The one or more non-transitory computer-readable storage media according to claim 197, wherein the model is trained using liquid chromatographic and mass spectrometry data obtained from a plurality of biological samples.

284. The one or more non-transitory computer-readable storage media according to any of claims 197 or 283, wherein the biological sample comprises a plurality of distinct biological samples obtained from one or more subjects at one or more times.

285. The one or more non-transitory computer-readable storage media of any one of claims 197 to 284, wherein the instructions, when executed by the at least one processor, cause the at least one processor to perform further operations comprising:
obtaining second liquid chromatographic and mass spectrometry data from a second biological sample;
preprocessing the second liquid chromatographic and mass spectrometry data from the second biological sample into one or more arrays, wherein the arrays may be indexed based on mass-to-charge ratio and retention time;
generating a tensor data structure for each precursor of the training analytes, wherein each tensor comprises a three-dimensional array of excerpts of the second preprocessed liquid chromatographic and mass spectrometry data comprising intensity data centered around the expected mass-to-charge ratios and the predicted retention times of the isotopes and product ions of the precursor; and
further training the model using the tensors corresponding to the precursors of the training analytes associated with the second biological sample to estimate a presence of precursors corresponding to analytes.

286. The one or more non-transitory computer-readable storage media according to any of claims 199 to 200, wherein the model is trained using liquid chromatographic and mass spectrometry data obtained from a plurality of biological samples.

287. The one or more non-transitory computer-readable storage media according to any of claims 199 to 200 or 286, wherein the biological sample comprises a plurality of distinct biological samples obtained from one or more subjects at one or more times.

288. The one or more non-transitory computer-readable storage media according to any of claims 199 to 200 or 286 to 287, wherein the instructions, when executed by the at least one processor, cause the at least one processor to perform further operations comprising:
obtaining second liquid chromatographic and mass spectrometry data from a second biological sample;
preprocessing the second liquid chromatographic and mass spectrometry data from the second biological sample into one or more arrays, wherein the arrays may be indexed based on mass-to-charge ratio and retention time;
generating a tensor data structure for each precursor of the training analytes, wherein each tensor comprises a three-dimensional array of excerpts of the second preprocessed liquid chromatographic and mass spectrometry data comprising intensity data centered around the expected mass-to-charge ratios and the predicted retention times of the isotopes and product ions of the precursor; and
further training the model using the tensors corresponding to the precursors of the training analytes associated with the second biological sample to estimate levels of precursors corresponding to analytes.

289. The one or more non-transitory computer-readable storage media according to claim 205, wherein the model is trained using liquid chromatographic and mass spectrometry data obtained from a plurality of biological samples suspected of exhibiting the condition.

290. The one or more non-transitory computer-readable storage media according to any of claim 205 or 289, wherein the model is trained using liquid chromatographic and mass spectrometry data obtained from a plurality of biological samples suspected of not exhibiting the condition.

291. The one or more non-transitory computer-readable storage media according to any of claims 205 or 289 to 290, wherein the first biological sample comprises a plurality of distinct biological samples suspected of exhibiting the condition and obtained from one or more subjects at one or more times.

292. The one or more non-transitory computer-readable storage media according to any of claims 205 or 289 to 291, wherein the second biological sample comprises a plurality of distinct biological samples suspected of not exhibiting the condition and obtained from one or more subjects at one or more times.

293. The one or more non-transitory computer-readable storage media according to any of claims 205 or 289 to 291, wherein the instructions, when executed by the at least one processor, cause the at least one processor to perform further operations comprising:
obtaining third liquid chromatographic and mass spectrometry data from a third biological sample suspected of exhibiting the condition;
obtaining fourth liquid chromatographic and mass spectrometry data from a fourth biological sample suspected of not exhibiting the condition;
preprocessing the second liquid chromatographic and mass spectrometry data from the third and fourth biological sample into one or more arrays, wherein the arrays may be indexed based on mass-to-charge ratio and retention time;
generating a tensor data structure for each precursor of the training analytes, wherein each tensor comprises a three-dimensional array of excerpts of the third and fourth preprocessed liquid chromatographic and mass spectrometry data comprising intensity data centered around the expected mass-to-charge ratios and the predicted retention times of the isotopes and product ions of the precursor; and
further training the model using the tensors corresponding to the precursors of the training analytes associated with the third and fourth biological samples to estimate characteristics of the condition.
